SEQUENCE LISTING (93 sequences)

SEQ ID NO: 1 - DNA, 2296 bp, synthetic construct
  misc_feature 1..2296: note = pML23_CVB3-EGFP
  source 1..2296: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaaaaacaaaaaacaaaacgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 2 - DNA, 2562 bp, synthetic construct
  misc_feature 1..2562: note = pML24_CVB3-EGFP-
  source 1..2562: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaaaaacaaaaaacaaaacgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 3 - DNA, 2666 bp, synthetic construct
  misc_feature 1..2666: note = pML25_UTR-CVB3-E
  source 1..2666: mol_type = other DNA
taatacgactcactataggggatccagagcggccgctttttcagcaagattaagcccagggcagagccatctattgcttacatttgcttctgacacaactgtgttcactagcaacctcaaacagacaccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaaaaacaaaaaacaaaacgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 4 - DNA, 2296 bp, synthetic construct
  misc_feature 1..2296: note = pML26_CVB3-EGFP-
  source 1..2296: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 5 - DNA, 2562 bp, synthetic construct
  misc_feature 1..2562: note = pML27_CVB3-EGFP-
  source 1..2562: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 6 - DNA, 2562 bp, synthetic construct
  misc_feature 1..2562: note = pML28_CVB3-EGFP-
  source 1..2562: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 7 - DNA, 2134 bp, synthetic construct
  misc_feature 1..2134: note = pML29_CVB3-GLuc-
  source 1..2134: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaaaaaaacaaaaaacaaaacgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 8 - DNA, 2400 bp, synthetic construct
  misc_feature 1..2400: note = pML30_CVB3-GLuc-
  source 1..2400: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaaaaaaacaaaaaacaaaacgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 9 - DNA, 2134 bp, synthetic construct
  misc_feature 1..2134: note = pML31_CVB3-GLuc-
  source 1..2134: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaagtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 10 - DNA, 2400 bp, synthetic construct
  misc_feature 1..2400: note = pML32_CVB3-GLuc-
  source 1..2400: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 11 - DNA, 2400 bp, synthetic construct
  misc_feature 1..2400: note = pML33_CVB3-GLuc-
  source 1..2400: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaagtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 12 - DNA, 2296 bp, synthetic construct
  misc_feature 1..2296: note = pML34_4xS1m-CVB3
  source 1..2296: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccggtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 13 - DNA, 2125 bp, synthetic construct
  misc_feature 1..2125: note = pML35_CVB3-EGFP-
  source 1..2125: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaaaaacaaaaaacaaaacaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaaggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 14 - DNA, 2391 bp, synthetic construct
  misc_feature 1..2391: note = pML36_CVB3-EGFP-
  source 1..2391: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaaaaacaaaaaacaaaacaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 15 - DNA, 2495 bp, synthetic construct
  misc_feature 1..2495: note = pML37_UTR-CVB3-E
  source 1..2495: mol_type = other DNA
taatacgactcactataggggatccagagcggccgctttttcagcaagattaagcccagggcagagccatctattgcttacatttgcttctgacacaactgtgttcactagcaacctcaaacagacaccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaaaaacaaaaaacaaaacaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 16 - DNA, 2125 bp, synthetic construct
  misc_feature 1..2125: note = pML38_CVB3-EGFP-
  source 1..2125: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 17 - DNA, 2391 bp, synthetic construct
  misc_feature 1..2391: note = pML39_CVB3-EGFP-
  source 1..2391: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 18 - DNA, 2391 bp, synthetic construct
  misc_feature 1..2391: note = pML40_CVB3-EGFP
  source 1..2391: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 19 - DNA, 1963 bp, synthetic construct
  misc_feature 1..1963: note = pML41_CVB3-GLuc
  source 1..1963: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaaaaaaacaaaaaacaaaacaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaaggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 20 - DNA, 2229 bp, synthetic construct
  misc_feature 1..2229: note = pML42_CVB3-GLuc-
  source 1..2229: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaaaaaaacaaaaaacaaaacaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 21 - DNA, 1963 bp, synthetic construct
  misc_feature 1..1963: note = pML43_CVB3-GLuc-
  source 1..1963: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 22 - DNA, 2333 bp, synthetic construct
  misc_feature 1..2333: note = pML44_CVB3-GLuc
  source 1..2333: mol_type = other DNA
taatacgactcactataggggatccagagcggccgctttttcagcaagattaagcccagggcagagccatctattgcttacatttgcttctgacacaactgtgttcactagcaacctcaaacagacaccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 23 - DNA, 2125 bp, synthetic construct
  misc_feature 1..2125: note = pML45_tS1m-CVB3-
  source 1..2125: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaaaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 24 - DNA, 1798 bp, synthetic construct
  misc_feature 1..1798: note = pML46_CVB3-GLuc
  source 1..1798: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 25 - DNA, 1960 bp, synthetic construct
  misc_feature 1..1960: note = pML47_CVB3-EGFP-
  source 1..1960: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 26 - DNA, 2308 bp, synthetic construct
  misc_feature 1..2308: note = pML48_CVB3-EGFP
  source 1..2308: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtaagtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcat

SEQ ID NO: 27 - DNA, 2146 bp, synthetic construct
  misc_feature 1..2146: note = pML49_CVB3-GLuc-
  source 1..2146: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtaagtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcat

SEQ ID NO: 28 - DNA, 2122 bp, synthetic construct
  misc_feature 1..2122: note = pML50_CVB3-EGFP-
  source 1..2122: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgcca

SEQ ID NO: 29 - DNA, 1960 bp, synthetic construct
  misc_feature 1..1960: note = pML51_CVB3-Gluc-
  source 1..1960: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgcca

SEQ ID NO: 30 - DNA, 1958 bp, synthetic construct
  misc_feature 1..1958: note = pML75_EGFP-CVB3-
  source 1..1958: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaacaaaaaacaaaaaaaacaaaaaaaaaaccaaaaaaacaaaacacaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 31 - DNA, 1949 bp, synthetic construct
  misc_feature 1..1949: note = pML76_EGFP-CVB3-
  source 1..1949: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgacaaaaaaaacaaaaaaaaaaccaaaaaaacaaaacacaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 32 - DNA, 1940 bp, synthetic construct
  misc_feature 1..1940: note = pML77_EGFP-CVB3
  source 1..1940: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgacaaaaaaaaaaccaaaaaaacaaaacacaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 33 - DNA, 1907 bp, synthetic construct
  misc_feature 1..1907: note = pML78_EGFP-CVB3-
  source 1..1907: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgtcgccggaaacgcaatagccgacaaaaaacaaaaaaaacaaaaaaaaaaccaaaaaaacaaaacacaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaaaaaacaaaaaacaaaacggctattatgcgttaccggcgacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 34 - DNA, 2121 bp, synthetic construct
  misc_feature 1..2121: note = pML79_EGFP-CVB3-
  source 1..2121: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgacaaaaaacaaaaaaaacaaaaaaaaaaccaaaaaaacaaaacacaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaaaaaacaaaaaacaaaacaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaaggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 35 - DNA, 2121 bp, synthetic construct
  misc_feature 1..2121: note = pML80_EGFP-CVB3-
  source 1..2121: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgacaaaaaacaaaaaaaacaaaaaaaaaaccaaaaaaacaaaacacaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 36 - DNA, 2102 bp, synthetic construct
  misc_feature 1..2102: note = pML81_EGFP-CVB3-
  source 1..2102: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgcaaaaaacaaaaaaaacaaaaaaaaaaccaaaaaaacaaaacacaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacgaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgcca

SEQ ID NO: 37 - DNA, 2120 bp, synthetic construct
  misc_feature 1..2120: note = pML82_EGFP-CVB3
  source 1..2120: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgcaaaaaacaaaaaaaacaaaaaaaaaaccaaaaaaacaaaacacaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaaaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaagaccagtggacaatcgacg

SEQ ID NO: 38 - DNA, 2120 bp, synthetic construct
  misc_feature 1..2120: note = pML83_tS1m-EGFP-
  source 1..2120: mol_type = other DNA
taatacgactcactataggggatccgggagaccctcgacaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaacgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgcaaaaaacaaaaaaaacaaaaaaaaaaccaaaaaaacaaaacacaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 39 - DNA, 2285 bp, synthetic construct
  misc_feature 1..2285: note = pML84_tS1m-EGFP-
  source 1..2285: mol_type = other DNA
taatacgactcactataggggatccgggagaccctcgaccgtcgattgtccactggtcaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaaaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgcaaaaaacaaaaaaaacaaaaaaaaaaccaaaaaaacaaaacacaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaaaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaagaccagtggacaatcgacg

SEQ ID NO: 40 - DNA, 2267 bp, synthetic construct
  misc_feature 1..2267: note = pML85_tS1m-EGFP-
  source 1..2267: mol_type = other DNA
taatacgactcactataggggatccgggagaccctcgacaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaacgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgcaaaaaacaaaaaaaacaaaaaaaaaaccaaaaaaacaaaacacaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacgaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgcca

SEQ ID NO: 41 - DNA, 2120 bp, synthetic construct
  misc_feature 1..2120: note = pML86_EGFP-tS1m-
  source 1..2120: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgcaaaaaacaaaaaaaacaaaaaaaaaaccaaaaaaacaaaacacaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 42 - DNA, 2210 bp, synthetic construct
  misc_feature 1..2210: note = pML87_EGFP-tS1m-
  source 1..2210: mol_type = other DNA
taatacgactcactataggggatccgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgcaaaaaacaaaaaaaacaaaaaaaaaaccaaaaaaacaaaacacaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacaaaaaacaaaaaaaacaaaaaaaaaaccaaaaaaacaaaacacaaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacg

SEQ ID NO: 43 - DNA, 2128 bp, synthetic construct
  misc_feature 1..2128: note = pML89_tS1m-CVB3-
  source 1..2128: mol_type = other DNA
taatacgactcactataggggatccgggagaccctcgaccgtcgattgtccactggtcaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaaaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaaaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaagaccagtggacaatcgacg

SEQ ID NO: 44 - DNA, 19 bp, synthetic construct
  misc_feature 1..19: note = T7 promoter
  source 1..19: mol_type = other DNA
taatacgactcactatagg

SEQ ID NO: 45 - DNA, 19 bp, synthetic construct
  misc_feature 1..19: note = 5 external homology arm
  source 1..19: mol_type = other DNA
cgtcgattgtccactggtc

SEQ ID NO: 46 - DNA, 20 bp, synthetic construct
  misc_feature 1..20: note = 5 internal homology arm
  source 1..20: mol_type = other DNA
cgccggaaacgcaatagccg

SEQ ID NO: 47 - DNA, 21 bp, synthetic construct
  misc_feature 1..21: note = 3 internal homology arm
  source 1..21: mol_type = other DNA
ggctattatgcgttaccggcg

SEQ ID NO: 48 - DNA, 19 bp, synthetic construct
  misc_feature 1..19: note = 3 external homology arm
  source 1..19: mol_type = other DNA
gaccagtggacaatcgacg

SEQ ID NO: 49 - DNA, 182 bp, synthetic construct
  misc_feature 1..182: note = Ana2.0 PIE 3
  source 1..182: mol_type = other DNA
aacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccaccccca

SEQ ID NO: 50 - DNA, 130 bp, synthetic construct
  misc_feature 1..130: note = Ana2.0 PIE 5
  source 1..130: mol_type = other DNA
agacgctacggacttaaataattgagccttaaagaagaaattctttaagtggatgctctcaaactcagggaaacctaaatctagttatagacaaggcaatcctgagccaagccgaagtagtaattagtaa

SEQ ID NO: 51 - DNA, 741 bp, synthetic construct
  misc_feature 1..741: note = CVB3 IRES
  source 1..741: mol_type = other DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 52 - DNA, 60 bp, synthetic construct
  misc_feature 1..60: note = S1m aptamer
  source 1..60: mol_type = other DNA
atgcggccgccgaccagaatcatgcaagtgcgtaagatagtcgcgggtcggcggccgcat

SEQ ID NO: 53 - DNA, 321 bp, synthetic construct
  misc_feature 1..321: note = 4xS1m aptamer
  source 1..321: mol_type = other DNA
atgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcat

SEQ ID NO: 54 - DNA, 134 bp, synthetic construct
  misc_feature 1..134: note = tRNA-S1m aptamer
  source 1..134: mol_type = other DNA
gcccggatagctcagtcggtagagcagcggcctatgcggccgccgaccagaatcatgcaagtgcgtaagatagtcgcgggtcggcggccgcattcgaggccgcgtccagggttcaagtccctgttcgggcgcca

SEQ ID NO: 55 - DNA, 720 bp, synthetic construct
  misc_feature 1..720: note = EGFP
  source 1..720: mol_type = other DNA
atggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 56 - DNA, 558 bp, synthetic construct
  misc_feature 1..558: note = hGLuc
  source 1..558: mol_type = other DNA
atgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaa

SEQ ID NO: 57 - DNA, 104 bp, synthetic construct
  misc_feature 1..104: note = 5 UTR
  source 1..104: mol_type = other DNA
agagcggccgctttttcagcaagattaagcccagggcagagccatctattgcttacatttgcttctgacacaactgtgttcactagcaacctcaaacagacacc

SEQ ID NO: 58 - DNA, 266 bp, synthetic construct
  misc_feature 1..266: note = 3 UTR
  source 1..266: mol_type = other DNA
agctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgc

SEQ ID NO: 59 - DNA, 50 bp, synthetic construct
  misc_feature 1..50: note = 5 spacer poly(A) site 1
  source 1..50: mol_type = other DNA
aaaaacaaaaaacaaaaaaaacaaaaaaaaaaccaaaaaaacaaaacaca

SEQ ID NO: 60 - DNA, 19 bp, synthetic construct
  misc_feature 1..19: note = 3spacer poly(A) site 2
  source 1..19: mol_type = other DNA
aaaaaacaaaaaacaaaac

SEQ ID NO: 61 - DNA, 131 bp, synthetic construct
  misc_feature 1..131: note = Ana2.0 3 intron
  source 1..131: mol_type = other DNA
aacaatagatgacttacaactaatcggaaggtgcagagactcgacgggagctaccctaacgtcaagacgagggtaaagagagagtccaattctcaaagccaataggcagtagcgaaagctgcaagagaatg

SEQ ID NO: 62 - DNA, 51 bp, synthetic construct
  misc_feature 1..51: note = Ana2.0 3 exon
  source 1..51: mol_type = other DNA
aaaatccgttgaccttaaacggtcgtgtgggttcaagtccctccaccccca

SEQ ID NO: 63 - DNA, 115 bp, synthetic construct
  misc_feature 1..115: note = Ana2.0 5 intron
  source 1..115: mol_type = other DNA
aaataattgagccttaaagaagaaattctttaagtggatgctctcaaactcagggaaacctaaatctagttatagacaaggcaatcctgagccaagccgaagtagtaattagtaa

SEQ ID NO: 64 - DNA, 15 bp, synthetic construct
  misc_feature 1..15: note = Ana2.0 5 exon
  source 1..15: mol_type = other DNA
agacgctacggactt

SEQ ID NO: 65 - RNA, 60 nt, synthetic construct
  source 1..60: mol_type = other RNA
atgcggccgccgaccagaatcatgcaagtgcgtaagatagtcgcgggtcggcggccgcat

SEQ ID NO: 66 - RNA, 321 nt, synthetic construct
  source 1..321: mol_type = other RNA
atgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcat

SEQ ID NO: 67 - RNA, 134 nt, synthetic construct
  source 1..134: mol_type = other RNA
gcccggatagctcagtcggtagagcagcggcctatgcggccgccgaccagaatcatgcaagtgcgtaagatagtcgcgggtcggcggccgcattcgaggccgcgtccagggttcaagtccctgttcgggcgcca

SEQ ID NO: 68 - RNA, 19 nt, synthetic construct
  source 1..19: mol_type = other RNA
taatacgactcactatagg

SEQ ID NO: 69 - RNA, 19 nt, synthetic construct
  source 1..19: mol_type = other RNA
cgtcgattgtccactggtc

SEQ ID NO: 70 - RNA, 20 nt, synthetic construct
  source 1..20: mol_type = other RNA
cgccggaaacgcaatagccg

SEQ ID NO: 71 - RNA, 21 nt, synthetic construct
  source 1..21: mol_type = other RNA
ggctattatgcgttaccggcg

SEQ ID NO: 72 - RNA, 19 nt, synthetic construct
  source 1..19: mol_type = other RNA
gaccagtggacaatcgacg

SEQ ID NO: 73 - RNA, 182 nt, synthetic construct
  source 1..182: mol_type = other RNA
aacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccaccccca

SEQ ID NO: 74 - RNA, 130 nt, synthetic construct
  source 1..130: mol_type = other RNA
agacgctacggacttaaataattgagccttaaagaagaaattctttaagtggatgctctcaaactcagggaaacctaaatctagttatagacaaggcaatcctgagccaagccgaagtagtaattagtaa

SEQ ID NO: 75 - RNA, 741 nt, synthetic construct
  source 1..741: mol_type = other RNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 76 - RNA, 104 nt, synthetic construct
  source 1..104: mol_type = other RNA
agagcggccgctttttcagcaagattaagcccagggcagagccatctattgcttacatttgcttctgacacaactgtgttcactagcaacctcaaacagacacc

SEQ ID NO: 77 - RNA, 266 nt, synthetic construct
  source 1..266: mol_type = other RNA
agctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgc

SEQ ID NO: 78 - RNA, 50 nt, synthetic construct
  source 1..50: mol_type = other RNA
aaaaacaaaaaacaaaaaaaacaaaaaaaaaaccaaaaaaacaaaacaca

SEQ ID NO: 79 - RNA, 19 nt, synthetic construct
  source 1..19: mol_type = other RNA
aaaaaacaaaaaacaaaac

SEQ ID NO: 80 - RNA, 131 nt, synthetic construct
  source 1..131: mol_type = other RNA
aacaatagatgacttacaactaatcggaaggtgcagagactcgacgggagctaccctaacgtcaagacgagggtaaagagagagtccaattctcaaagccaataggcagtagcgaaagctgcaagagaatg

SEQ ID NO: 81 - RNA, 51 nt, synthetic construct
  source 1..51: mol_type = other RNA
aaaatccgttgaccttaaacggtcgtgtgggttcaagtccctccaccccca

SEQ ID NO: 82 - RNA, 115 nt, synthetic construct
  source 1..115: mol_type = other RNA
aaataattgagccttaaagaagaaattctttaagtggatgctctcaaactcagggaaacctaaatctagttatagacaaggcaatcctgagccaagccgaagtagtaattagtaa

SEQ ID NO: 83 - RNA, 15 nt, synthetic construct
  source 1..15: mol_type = other RNA
agacgctacggactt

SEQ ID NO: 84 - RNA, 56 nt, synthetic construct
  source 1..56: mol_type = other RNA
gggtacgctcaggtatattggcgccttcgtggaatgtcagtgcctggacgtgcagt

SEQ ID NO: 85 - RNA, 80 nt, synthetic construct
  source 1..80: mol_type = other RNA
gggacgctcacgtacgctcacgtccgatcgatactggtatattggcgccttcgtggaatgtcagtgcctggacgtgcagt

SEQ ID NO: 86 - RNA, 37 nt, synthetic construct
  source 1..37: mol_type = other RNA
gggtatattggcgccttcgtggaatgtcagtgcctgg

SEQ ID NO: 87 - RNA, 34 nt, synthetic construct
  source 1..34: mol_type = other RNA
ggccaacatgaggatcacccatgtctgcagggcc

SEQ ID NO: 88 - RNA, 18 nt, synthetic construct
  source 1..18: mol_type = other RNA
acatgaggatcacccatg

SEQ ID NO: 89 - RNA, 19 nt, synthetic construct
  source 1..19: mol_type = other RNA
acatgaggatcacccatgt

SEQ ID NO: 90 - RNA, 40 nt, synthetic construct
  source 1..40: mol_type = other RNA
tcatgcaagtgcgtaagatagtcgcgggccgggggcgtat

SEQ ID NO: 91 - RNA, 40 nt, synthetic construct
  source 1..40: mol_type = other RNA
gtccgagtaatttacgttttgatacggttgcggaacttgc

SEQ ID NO: 92 - RNA, 23 nt, synthetic construct
  source 1..23: mol_type = other RNA
gaagggacggtgcggagaggaga

SEQ ID NO: 93 - RNA, 28 nt, synthetic construct
  source 1..28: mol_type = other RNA
agcttatccattgcatctcggatgagct

(19) United States
(12) Patent Application Publication
(10) Pub. No.: US 2025/0223592 A1
(43) Pub. Date: Jul. 10, 2025

(54) COMPOSITIONS AND METHODS FOR CIRCULAR RNA AFFINITY PURIFICATION

(71) Applicant: SANOFI PASTEUR INC., Swiftwater, PA (US)

(72) Inventors: Jianping CUI, Cambridge, MA (US); Michelle Marie LOGSDON, Cambridge, MA (US)

(21) Appl. No.: 18/978,229

(22) Filed: Dec. 12, 2024

Related U.S. Application Data
(63) Continuation of application No. PCT/EP2023/066315, filed on Jun. 16, 2023.

(30) Foreign Application Priority Data
Jun. 17, 2022 (EP) 22305884.3
Oct. 6, 2022 (EP) 22306497.3

(51) Int. Cl. C12N 15/113; C12N 15/10; C12N 15/115

(52) U.S. Cl. CPC C12N 15/113; C12N 15/101; C12N 15/115; C12N 2310/12; C12N 2310/16; C12N 2310/30; C12N 2310/532

(57) ABSTRACT

The present disclosure provides for circular RNA (circRNA) compositions and methods purification and use of the same. In particular, the disclosure relates to compositions and methods of making and using circRNA comprising one or more aptamers which specifically bind an affinity ligand.

### RELATED APPLICATIONS

This application is a continuation of International Patent Application No. PCT/EP2023/066315, filed Jun. 16, 2023, which claims priority to European Patent Application Nos. 22305884.3, filed Jun. 17, 2022, and 22306497.3, filed Oct. 6, 2022, the entire contents of which are incorporated herein by reference.

### SEQUENCE LISTING

The instant application contains a Sequence Listing which has been submitted electronically in XML format and is hereby incorporated by reference in its entirety. Said XML file, created on Dec. 12, 2024, is named 759390_SA9-328PCCON_ST26.xml and is 206,994 bytes in size.

### BACKGROUND OF THE DISCLOSURE

Exogenous circularized RNAs (circRNAs) containing a protein coding region are emerging as a valuable a molecular tool and an alternative to messenger RNA (mRNA) therapeutics. CircRNAs are single-stranded and characterized by a covalently closed structure. In contrast to linear RNA, circRNAs have elevated stability, a significantly longer half-life, and are resistant to degradation by exonucleases. Uses of exogenous circRNAs include (1) the overexpression of native circRNAs, (2) the engineering of in vitro produced circRNA as a substitute to existing linear mRNA delivery, and/or (3) as described herein as part of a production and purification method for linear and/or circular RNA.

Methods for efficiently purifying exogenous circRNA remain a significant obstacle that must be overcome before the protein coding potential of circRNA can be fully realized. This is partly due to the different types and combinations of undesired contaminants in a sample that need to be separated from a pure sample of circRNA. Such contaminants are typically components and by-products of any upstream processes, for example RNA manufacturing and circularization conditions. The sample typically contains the desired circRNA alongside various contaminants such as linear precursor RNA, nicked circular RNA, double stranded RNA, triphosphate-RNA, free nucleotides, endotoxins, and solvents.

There remains a need for more effective, reliable, and safer methods of purifying circRNA from large scale manufacturing processes for potential therapeutic applications which are also economical in terms of the number of steps, the complexity of the steps, and the resources used in the steps.

### BRIEF SUMMARY OF THE DISCLOSURE

In one aspect, the disclosure provides a circular RNA comprising a protein coding region and at least one RNA aptamer.

In certain embodiments, an internal ribosome entry site (IRES) is positioned at the 5′ end of the protein coding region.

In certain embodiments, an IRES is positioned at the 3′ end of the protein coding region.

In certain embodiments, the IRES is derived from Coxsackievirus B3 (CVB3), Encephalomyocarditis virus (EMCV), Dicistroviruses, hepatitis C virus (HCV), poliovirus (PV), enterovirus 71 (EV71), human rhinovirus (HRV), foot-and-mouth disease virus (FMDV), or synthetic IRES.

In certain embodiments, the IRES comprises a polynucleotide sequence of SEQ ID NO: 75.

In certain embodiments, the protein coding region encodes at least one polypeptide or peptide.

In certain embodiments, the polypeptide is a biologically active polypeptide, a therapeutic polypeptide, or an antigenic polypeptide.

In certain embodiments, the circular RNA comprises at least one 5′ internal homology arm and at least one 3′ internal homology arm.

In certain embodiments, the 5′ internal homology arm is about 5 to about 50 nucleotides in length.

In certain embodiments, the 5′ internal homology arm comprises the nucleotide sequence of SEQ ID NO: 70.

In certain embodiments, the 3′ internal homology arm is about 5 to about 50 nucleotides in length.

In certain embodiments, the 3′ internal homology arm comprises the nucleotide sequence of SEQ ID NO: 71.

In certain embodiments, the circular RNA comprises at least one 3′ exon element.

In certain embodiments, the 3′ exon element comprises the nucleotide sequence of SEQ ID NO: 81.

In certain embodiments, the circular RNA comprises at least one 5′ exon element.

In certain embodiments, the 5′ exon element comprises the nucleotide sequence of SEQ ID NO: 83.

In certain embodiments, the circular RNA comprises at least one spacer sequence.

In certain embodiments, the spacer sequence is about 5 to about 75 nucleotides in length.

In certain embodiments, the spacer sequence comprises the nucleotide sequence of SEQ ID NO: 78 or 79.

In certain embodiments, the spacer sequence is positioned at one or both of a 5′ end and 3′ end of any one of the following elements: the protein coding region, the IRES, the 5′ internal homology arm, the 3′ internal homology arm, the 5′ exon element, and the 3′ exon element.

In certain embodiments, the circular RNA comprises the following elements, from 5′ to 3′: a) the 3′ exon element, b) the 5′ internal homology arm, c) the spacer sequence, d) the IRES, e) the protein coding region, f) the spacer sequence, g) the 3′ internal homology arm, and h) the 5′ exon element.

In certain embodiments, the circular RNA comprises the following elements, from 5′ to 3′: a) the 3′ exon element, b) the 5′ internal homology arm, c) the spacer sequence, d) the protein coding region, e) the IRES, f) the spacer sequence, g) the 3′ internal homology arm, and h) the 5′ exon element.

In certain embodiments, the at least one RNA aptamer is positioned at a 5′ end or a 3′ end of any one of elements a)-h).

In certain embodiments, the circular RNA contains at least one 5′ untranslated region (5′ UTR), at least one 3′ untranslated region (3′ UTR), and/or at least one polyadenylation (polyA) sequence.

In certain embodiments, the 5′ UTR, the 3′ UTR, and/or the polyA sequence are spacer sequences.

In certain embodiments, the RNA aptamer is embedded in an RNA scaffold.

In certain embodiments, the RNA scaffold comprises at least one secondary structure motif.

In certain embodiments, the secondary structure motif is a tetraloop, a pseudoknot, or a stem-loop.

In certain embodiments, the RNA scaffold comprises at least one tertiary structure.

In certain embodiments, the secondary structure motif and/or tertiary structure are nuclease resistant.

In certain embodiments, the RNA scaffold comprises a transfer RNA (tRNA).

In certain embodiments, the RNA aptamer is embedded in a tRNA hairpin loop of the tRNA.

In certain embodiments, the RNA aptamer is embedded in a tRNA anticodon loop of the tRNA.

In certain embodiments, the RNA aptamer is embedded in a tRNA D loop of the tRNA.

In certain embodiments, the RNA aptamer is S1m, Sm, or a derivative or fragment thereof.

In certain embodiments, the circular RNA comprises between one to four RNA aptamers.

In certain embodiments, the RNA aptamers are identical.

In certain embodiments, at least one of the RNA aptamers is distinct.

In certain embodiments, the RNA aptamer is synthetically derived.

In certain embodiments, the RNA aptamer is a split aptamer or an X-aptamer.

In certain embodiments, the RNA aptamer is naturally-derived.

In certain embodiments, the RNA aptamer is derived from a hairpin RNA, a tRNA, or a riboswitch.

In certain embodiments, the RNA aptamer binds to an affinity ligand.

In certain embodiments, the affinity ligand comprises protein A, protein G, streptavidin, glutathione, dextran, or a fluorescent molecule.

In certain embodiments, the affinity ligand comprises streptavidin.

In certain embodiments, the affinity ligand is immobilized on a chromatography resin.

In certain embodiments, the at least one RNA aptamer is positioned: a) before the 3′ exon element, b) between the 3′ exon element and the 5′ internal homology arm, c) between the 5′ internal homology arm and the 5′ spacer sequence, d) between the 5′ spacer sequence and the IRES, e) between the protein coding region and the 3′ spacer sequence, f) between the 3′ spacer sequence and the 3′ internal homology arm, g) between the 3′ internal homology arm and the 5′ exon element, h) after the 5′ exon element, i) between the 3′ exon and the IRES, and/or j) between the IRES and the 5′ exon element.

In certain embodiments, the at least one RNA aptamer is positioned: a) before the 3′ exon element, b) between the 3′ exon element and the 5′ internal homology arm, c) between the 5′ internal homology arm and the 5′ spacer sequence, d) between the 5′ spacer sequence and the protein coding region, e) between the IRES and the 3′ spacer sequence, f) between the 3′ spacer sequence and the 3′ internal homology arm, g) between the 3′ internal homology arm and the 5′ exon element, h) after the 5′ exon element, i) between the 3′ exon and the protein coding region, and/or j) between the protein coding region and the 5′ exon element.

In certain embodiments, the RNA aptamer comprises the nucleotide sequence of SEQ ID NO: 65 or 66.

In certain embodiments, the RNA aptamer comprises the nucleotide sequence of SEQ ID NO: 84. In certain embodiments, the RNA aptamer comprises the nucleotide sequence of SEQ ID NO: 85. In certain embodiments, the RNA aptamer comprises the nucleotide sequence of SEQ ID NO: 86. In certain embodiments, the RNA aptamer comprises the nucleotide sequence of SEQ ID NO: 87. In certain embodiments, the RNA aptamer comprises the nucleotide sequence of SEQ ID NO: 88. In certain embodiments, the RNA aptamer comprises the nucleotide sequence of SEQ ID NO: 89. In certain embodiments, the RNA aptamer comprises the nucleotide sequence of SEQ ID NO: 90. In certain embodiments, the RNA aptamer comprises the nucleotide sequence of SEQ ID NO: 91. In certain embodiments, the RNA aptamer comprises the nucleotide sequence of SEQ ID NO: 92. In certain embodiments, the RNA aptamer comprises the nucleotide sequence of SEQ ID NO: 93.

In certain embodiments, the RNA aptamer embedded tRNA comprises the nucleotide sequence of SEQ ID NO: 67.

In certain embodiments, the RNA aptamer is about 30-200 nucleotides in length.

In certain embodiments, the RNA aptamer is about 50-200 nucleotides in length.

In certain embodiments, the RNA aptamer is not a histone stem-loop.

In certain embodiments, the circular RNA comprises at least one chemical modification.

In certain embodiments, the chemical modification is pseudouridine, N1-methylpseudouridine, 2-thiouridine, 4′-thiouridine, 5-methylcytosine, 2-thio-I-methyl-1-deaza-pseudouridine, 2-thio-I-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-I-methyl-pseudouridine, 4-thio-pseudouridine, 5-aza-uridine, dihydropseudouridine, 5-methyluridine, 5-methyluridine, 5-methoxyuridine, 2′-O-methyl uridine, or N6-methyladenosine.

In certain embodiments, the chemical modification is pseudouridine, N1-methylpseudouridine, 5-methylcytosine, 5-methoxyuridine, N6-methyladenosine or a combination thereof.

In certain embodiments, the chemical modification is N1-methylpseudouridine.

In another aspect, the disclosure provides a linear precursor RNA comprising at least a self-splicing ribozyme and a protein coding region, wherein the linear precursor RNA comprises at least one RNA aptamer.

In certain embodiments, the self-splicing ribozyme comprises at least two catalytic subunits.

In certain embodiments, the self-splicing ribozyme catalytic subunits derive from either a group I intron or a group II intron RNA transcript or a fragment thereof.

In certain embodiments, the self-splicing ribozyme catalytic subunits derive from a permuted intron-exon (PIE) sequence from Cyanobacterium anabaena pre-tRNA-Leu gene, T4 phage Td gene, or Tetrahymena pre-rRNA.

In certain embodiments, the catalytic activity of the two subunits results in a circularized RNA.

In certain embodiments, the linear precursor RNA comprises the following elements, from 5′ to 3′: a) a 5′ external homology arm, b) a 3′ self-splicing PIE fragment, c) a 5′ internal homology arm, d) a 5′ spacer sequence, e) an internal ribosome entry site (IRES) f) a protein coding region, g) a 3′ spacer sequence, h) a 3′ internal homology arm, i) a 5′ self-splicing PIE fragment, and j) a 3′ external homology arm, wherein the RNA aptamer is present at one or both of the 5′ end or 3′ end of any one of elements a)-j).

In certain embodiments, the linear precursor RNA comprises the following elements, from 5′ to 3′: a) a 5′ external homology arm, b) a 3′ self-splicing PIE fragment, c) a 5′ internal homology arm, d) a 5′ spacer sequence, e) a protein coding region, f) an IRES, g) a 3′ spacer sequence, h) a 3′ internal homology arm, i) a 5′ self-splicing PIE fragment, and j) a 3′ external homology arm, wherein the RNA aptamer is present at one or both of the 5′ end or 3′ end of any one of elements a)-j).

In certain embodiments, the 5′ external homology arm and the 3′ external homology arm comprises the nucleotide sequence of SEQ ID NO: 69 or SEQ ID NO: 72.

In certain embodiments, the 5′ external homology arm and the 3′ external homology arm are each independently about 5 to about 50 nucleotides in length.

In certain embodiments, the 5′ self-splicing PIE fragment comprises the nucleotide sequence of SEQ ID NO: 74.

In certain embodiments, the 5′ internal homology arm comprises the nucleotide sequence of SEQ ID NO: 70.

In certain embodiments, the 5′ internal homology arm is about 5 to about 50 nucleotides in length.

In certain embodiments, the 5′ spacer and the 3′ spacer comprises the nucleotide sequence of SEQ ID NO: 78 or SEQ ID NO: 79.

In certain embodiments, the 5′ spacer and the 3′ spacer are each independently about 5 to 75 nucleotides in length

In certain embodiments, the 3′ self-splicing PIE fragment comprises the nucleotide sequence of SEQ ID NO: 73.

In certain embodiments, the IRES is derived from Coxsackievirus B3 (CVB3), Encephalomyocarditis virus (EMCV), Dicistroviruses, hepatitis C virus (HCV), poliovirus (PV), enterovirus 71 (EV71), human rhinovirus (HRV), foot-and-mouth disease virus (FMDV), or synthetic IRES.

In certain embodiments, the IRES comprises the nucleotide sequence of SEQ ID NO: 75.

In certain embodiments, the linear precursor RNA comprises at least one 5′ untranslated region (5′ UTR), at least one 3′ untranslated region (3′ UTR), and/or a polyadenylation (polyA) sequence.

In certain embodiments, the protein coding region encodes at least one polypeptide.

In certain embodiments, the polypeptide is a biologically active polypeptide, a therapeutic polypeptide, or an antigenic polypeptide.

In certain embodiments, the RNA aptamer is embedded in an RNA scaffold.

In certain embodiments, the RNA scaffold comprises at least one secondary structure motif.

In certain embodiments, the secondary structure motif is a tetraloop, a pseudoknot, or a stem-loop.

In certain embodiments, the RNA scaffold comprises at least one tertiary structure.

In certain embodiments, the secondary structure motif and/or tertiary structure are nuclease resistant.

In certain embodiments, the RNA scaffold comprises a transfer RNA (tRNA).

In certain embodiments, the RNA aptamer is embedded in a tRNA hairpin loop of the tRNA.

In certain embodiments, the RNA aptamer is embedded in a tRNA anticodon loop of the tRNA.

In certain embodiments, the RNA aptamer is embedded in a tRNA D loop of the tRNA.

In certain embodiments, the RNA aptamer is S1m, Sm, or a derivative or fragment thereof.

In certain embodiments, the linear precursor RNA comprises between one to four RNA aptamers.

In certain embodiments, the RNA aptamers are identical.

In certain embodiments, at least one of the RNA aptamers is distinct.

In certain embodiments, the RNA aptamer is synthetically derived.

In certain embodiments, the RNA aptamer is a split aptamer or an X-aptamer.

In certain embodiments, the RNA aptamer is a split aptamer comprising a 5′ portion and a 3′ portion.

In certain embodiments, the 5′ portion of the split aptamer is positioned 3′ of the 5′ exon element and the 3′ portion of the split aptamer is positioned 5′ of the 3′ exon element.

In certain embodiments, the 5′ portion of the split aptamer is positioned 3′ of the 3′ internal homology arm and the 3′ portion of the split aptamer is positioned 5′ of the 5′ internal homology arm.

In certain embodiments, the split aptamer is reformed to a functional aptamer upon circularization of the linear precursor RNA.

In certain embodiments, the RNA aptamer is naturally-derived.

In certain embodiments, the RNA aptamer is derived from a hairpin RNA, a tRNA, or a riboswitch.

In certain embodiments, the RNA aptamer binds to an affinity ligand.

In certain embodiments, the affinity ligand comprises protein A, protein G, streptavidin, glutathione, dextran, or a fluorescent molecule.

In certain embodiments, the affinity ligand comprises streptavidin.

In certain embodiments, the affinity ligand is immobilized on a chromatography resin.

In certain embodiments, the at least one RNA aptamer is positioned: a) before the 5′ external homology arm, b) between the 5′ external homology arm and the 3′ self-splicing PIE fragment, c) between the 3′ self-splicing PIE fragment and the 5′ internal homology arm, d) between the 5′ internal homology arm and the 5′ spacer sequence, e) between the 5′ space sequence and the IRES, f) after the protein coding region but before the 3′ spacer sequence, g) between the 3′ spacer sequence and the 3′ internal homology arm, h) between the 3′ internal homology arm and the 5′ self-splicing PIE fragment, i) between the 5′ self-splicing PIE fragment and the 3′ external homology arm, and/or j) after the 3′ external homology arm.

In certain embodiments, at least one RNA aptamer is positioned: a) before the 5′ external homology arm, b) between the 5′ external homology arm and the 3′ self-splicing PIE fragment, c) between the 3′ self-splicing PIE fragment and the 5′ internal homology arm, d) between the 5′ internal homology arm and the 5′ spacer sequence, e) between the 5′ space sequence and the protein coding region, f) after the IRES but before the 3′ spacer sequence, g) between the 3′ spacer sequence and the 3′ internal homology arm, h) between the 3′ internal homology arm and the 5′ self-splicing PIE fragment, i) between the 5′ self-splicing PIE fragment and the 3′ external homology arm, and/or j) after the 3′ external homology arm.

In certain embodiments, the RNA aptamer comprises the nucleotide sequence of SEQ ID NO: 65 or 66.

In certain embodiments, the RNA aptamer comprises the nucleotide sequence of SEQ ID NO: 84. In certain embodiments, the RNA aptamer comprises the nucleotide sequence of SEQ ID NO: 85. In certain embodiments, the RNA aptamer comprises the nucleotide sequence of SEQ ID NO: 86. In certain embodiments, the RNA aptamer comprises the nucleotide sequence of SEQ ID NO: 87. In certain embodiments, the RNA aptamer comprises the nucleotide sequence of SEQ ID NO: 88. In certain embodiments, the RNA aptamer comprises the nucleotide sequence of SEQ ID NO: 89. In certain embodiments, the RNA aptamer comprises the nucleotide sequence of SEQ ID NO: 90. In certain embodiments, the RNA aptamer comprises the nucleotide sequence of SEQ ID NO: 91. In certain embodiments, the RNA aptamer comprises the nucleotide sequence of SEQ ID NO: 92. In certain embodiments, the RNA aptamer comprises the nucleotide sequence of SEQ ID NO: 93.

In certain embodiments, the RNA aptamer embedded tRNA comprises the nucleotide sequence of SEQ ID NO: 67.

In certain embodiments, the RNA aptamer is about 30-200 nucleotides in length.

In certain embodiments, the RNA aptamer is about 50-200 nucleotides in length.

In certain embodiments, the RNA aptamer is not a histone stem-loop.

In certain embodiments, the linear precursor RNA comprises at least one chemical modification.

In certain embodiments, the chemical modification is pseudouridine, N1-methylpseudouridine, 2-thiouridine, 4′-thiouridine, 5-methylcytosine, 2-thio-I-methyl-1-deaza-pseudouridine, 2-thio-I-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-I-methyl-pseudouridine, 4-thio-pseudouridine, 5-aza-uridine, dihydropseudouridine, 5-methyluridine, 5-methyluridine, 5-methoxyuridine, 2′-O-methyl uridine, or N6-methyladenosine.

In certain embodiments, the chemical modification is pseudouridine, N1-methylpseudouridine, 5-methylcytosine, 5-methoxyuridine, N6-methyladenosine, or a combination thereof.

In certain embodiments, the chemical modification is N1-methylpseudouridine.

In certain embodiments, the linear precursor RNA is synthesized using in vitro transcription (IVT)

In one aspect, the disclosure provides a circular RNA comprising a protein coding region and at least one RNA aptamer, wherein the circular RNA is formed from the linear precursor RNA described above.

In one aspect, the disclosure provides a circular RNA comprising a protein coding region, wherein the circular RNA is formed from the linear precursor RNA described above, and wherein the circular RNA lacks an RNA aptamer.

In one aspect, the disclosure provides a nucleic acid that encodes the linear precursor RNA described above.

In one aspect, the disclosure provides a vector comprising the nucleic acid described above.

In one aspect, the disclosure provides a host cell comprising the vector described above.

In one aspect, the disclosure provides a pharmaceutical composition comprising the circular RNA described above or the linear precursor RNA described above.

In one aspect, the disclosure provides a method of producing a circular RNA, comprising incubating the linear precursor RNA described above under conditions that result in the circularization of the linear precursor RNA.

In certain embodiments, the linear precursor RNA is incubated with GTP and Mg2+.

In certain embodiments, the linear precursor RNA is incubated with GTP and Mg2+ for a time sufficient to circularize the linear precursor RNA.

In certain embodiments, the GTP is present at a concentration of about 1 mM to about 15 mM.

In certain embodiments, the GTP is present at a concentration of about 2 mM.

In certain embodiments, the Mg2+ is present at a concentration of about 1 mM to about 50 mM.

In certain embodiments, the Mg2+ is present at a concentration of about 10 mM.

In one aspect, the disclosure provides a method of producing a plurality of circular RNA molecules, comprising incubating a plurality of linear precursor RNA molecules under conditions that result in the circularization of at least a portion of the linear precursor RNA molecules, wherein each linear precursor RNA molecule comprises the linear precursor RNA described above.

In certain embodiments, at least about 30% (i.e., about 30%, about 40%, about 50%, about 60%, about 70%, about 80%, about 90%, or 100%) of the linear precursor RNA molecules in the plurality are circularized.

In one aspect, the disclosure provides a method for purifying a circular RNA, comprising the steps of: (a) contacting a sample comprising the circular RNA described above with an affinity ligand that is immobilized on a chromatography resin, wherein the RNA aptamer comprises binding affinity for the affinity ligand; (b) eluting the circular RNA from the chromatography resin; and (c) purifying the circular RNA from the sample.

In one aspect, the disclosure provides a method for purifying a linear precursor RNA, comprising the steps of: (a) contacting a sample comprising the linear precursor RNA described above with an affinity ligand that is immobilized on a chromatography resin, wherein the RNA aptamer comprises binding affinity for the affinity ligand; (b) eluting the linear precursor RNA from the chromatography resin; and (c) purifying the linear precursor RNA from the sample.

In certain embodiments, the method comprises one or more washing steps between the contacting step (a) and the eluting step (b).

In one aspect, the disclosure provides a method of purifying a circular RNA, comprising the steps of: (a) contacting a sample comprising the circular RNA with an affinity ligand that is immobilized on a chromatography resin; (b) eluting the circular RNA from the chromatography resin; and (c) isolating the circular RNA from the sample, wherein the circular RNA comprises a protein coding region and at least one RNA aptamer, wherein the RNA aptamer comprises binding affinity for the affinity ligand.

In one aspect, the disclosure provides a method of purifying a linear precursor RNA, comprising the steps of: (a) contacting a sample comprising the linear precursor RNA with an affinity ligand that is immobilized on a chromatography resin; (b) eluting the linear precursor RNA from the chromatography resin; and (c) isolating the linear precursor RNA from the sample, wherein the linear precursor RNA comprises a protein coding region and at least one RNA aptamer, wherein the RNA aptamer comprises binding affinity for the affinity ligand.

In one aspect, the disclosure provides a method of purifying a circular RNA, comprising the steps of: (a) contacting a sample comprising a plurality of linear precursor RNA molecules and a plurality of circular RNA molecules with an affinity ligand that is immobilized on a chromatography resin; and (b) isolating the circular RNA molecules from the sample, wherein the linear precursor RNA molecules comprise a protein coding region and at least one RNA aptamer and wherein the RNA aptamer comprises binding affinity for the affinity ligand, and wherein the circular RNA molecules lack an RNA aptamer.

In certain embodiments, the circular RNA molecules do not bind the affinity ligand.

In certain embodiments, the circular RNA or linear precursor RNA is greater than or equal to 90% pure.

In one aspect, the disclosure provides a method of treating or preventing a disease or disorder, comprising administering to a subject in need thereof the pharmaceutical composition described above.

In one aspect, the disclosure provides a pharmaceutical composition comprising a plurality of circular RNA molecules, wherein at least about 90% of the circular RNA comprise a protein coding region and at least one RNA aptamer.

### BRIEF DESCRIPTION OF THE DRAWINGS/FIGURES

The foregoing and other features and advantages of the present disclosure will be more fully understood from the following detailed description of illustrative embodiments taken in conjunction with the accompanying drawings.

FIG. 1 left panel is a schematic diagram of the aptamer tagged linear precursor RNA that becomes circularized to form the aptamer tagged circRNA. The right panel shows streptavidin affinity binding during a purification process can occur with an aptamer tagged to a linear precursor RNA (top) or an aptamer tagged circRNA (bottom).

FIG. 2A depicts the plasmid map encoding the 4xS1m aptamer, the linear precursor RNA, and the PIE sequences used for RNA circularization. The plasmid elements are arranged in the following 5′ to 3′ order: a T7 promoter, a 5′ external homology arm, a 3″ Anabaena intron/exon fragment, a 5′ internal homology arm, a 5′ polyAC spacer, a CVB3 IRES, a protein coding region, a 3′ polyAC spacer, a 4xS1m aptamer, a 3′ internal homology arm, a 5″ Anabaena intron/exon fragment, and a 3′ external homology arm.

FIG. 2B depicts the plasmid map encoding the tRNA-S1m aptamer, the linear precursor RNA, and the PIE sequences used for RNA circularization. The plasmid elements are arranged in the following 5′ to 3′ order: a T7 promoter, a 5′ external homology arm, a 3′ Anabaena intron/exon fragment, a 5′ internal homology arm, a 5′ polyAC spacer, a CVB3 IRES, a protein coding region, a 3′ polyAC spacer, a 3′ internal homology arm, a 5′ Anabaena intron/exon fragment, a 3′ external homology arm, and a tRNA-S1m aptamer.

FIG. 2C depicts the control plasmid map which encodes the linear precursor RNA and PIE sequences used for RNA circularization but does not encode an aptamer. The plasmid elements are arranged in the following 5′ to 3′ order: a T7 promoter, a 5′ external homology arm, a 3′ Anabaena intron/exon fragment, a 5′ internal homology arm, a 5′ polyAC spacer, a CVB3 IRES, protein coding region, a 3′ polyAC spacer, a 3′ internal homology arm, a 5′ Anabaena intron/exon fragment, and a 3′ external homology arm.

FIG. 3 is an image of an agarose gel comparing the amount of RNA species (circular, precursor, or nicked) in the elution, unbound, and wash fractions after streptavidin Sepharose bead affinity purification of a 4xS1m aptamer tagged circRNA, a tRNA-S1m aptamer tagged circRNA, or a circRNA no aptamer control.

FIG. 4 is a bar graph that measures the elution, unbound, and wash fractions (wash 1 and wash 2) recovered after streptavidin Sepharose bead affinity purification of a 4xS1m aptamer tagged circRNA, a tRNA-S1m aptamer tagged circRNA, or a circRNA no aptamer control. The amount of recovered RNA measured is expressed as a percent of the input (i.e., the input being the sample of circRNA that did not undergo affinity purification).

FIG. 5 illustrates a design strategy to produce an aptamer tagged circRNA (left panel) and subsequent affinity purification (right panel) using a positive selection method. In the positive selection method, the linear precursor RNA will be flanked by a split aptamer which does not undergo affinity purification because the intact aptamer is required for binding to the affinity matrix. Upon circularization of the linear precursor RNA the intact aptamer will form allowing for binding to the affinity matrix.

FIG. 6 illustrates a design strategy to produce a circRNA (left panel) and subsequent affinity purification (right panel) using a negative selection method. In the negative selection method, the aptamer is localized outside of the 5′ end of 3′ intron or the 3′ end of 5′ intron of the linear precursor RNA such that the linear precursor RNA binds to the affinity matrix. Due to the positioning of the aptamer outside of the 5′ end of 3′ intron or the 3′ end of 5′ intron sequence the linear precursor RNA, upon circularization, the circRNA will not contain the aptamer and will not bind to the affinity matrix.

FIG. 7 is a bar graph that measures the elution, unbound, and wash recovered after streptavidin Sepharose bead affinity purification of a 4xS1m aptamer tagged linear precursor RNA (pML49), a tRNA-S1m aptamer tagged linear precursor RNA (pML50 and pML51), a no aptamer control (pML47), a 4xS1m aptamer tagged circRNA (pML26), and a tRNA-S1m aptamer tagged circRNA (pML38). The amount of recovered RNA measured is expressed as a percent of the input (i.e., the input being the total RNA in the sample).

FIG. 8A-8D are images of agarose gels comparing the amount of RNA species (circular, precursor, or nicked) in the elution, unbound, and wash fractions after streptavidin Sepharose bead affinity purification of a 4xS1m aptamer tagged linear precursor RNA (pML49, FIG. 8A), a tRNA-S1m aptamer tagged linear precursor RNA (pML50, FIG. 8B and pML51, FIG. 8C), and several controls (FIG. 8D).

FIG. 9A-9C are images of capillary electrophoresis traces comparing the amount of RNA species (circular, precursor, or nicked) in the input, elution, and unbound fractions after streptavidin Sepharose bead affinity purification of a tRNA-S1m aptamer tagged linear precursor RNA (pML50, FIG. 9A and pML51, FIG. 9B), and a 4xS1m aptamer tagged linear precursor RNA (pML49, FIG. 9C).

FIG. 10 depicts a bar graph of % linear precursor or circular/nicked RNA in the input, unbound, and wash fractions of a streptavidin Sepharose bead affinity purification.

FIG. 11A-11B depict % linear precursor or circular/nicked RNA and total yield (mg) in the input, unbound, and wash fractions of a streptavidin Sepharose bead affinity purification.

FIG. 12A depicts % linear precursor, circular/nicked RNA, and introns (combination of bound introns, 5′ intron, and 3′ intron) in the input, unbound, and wash fractions of a streptavidin Sepharose bead affinity purification. FIG. 12B depicts a schematic of a construct for IVT to produce a linear precursor RNA with a 5′ end and 3′ end aptamer.

FIG. 13 depicts % linear precursor or circular/nicked RNA of a large circRNA in the input and purified fractions of a streptavidin Sepharose bead affinity purification.

FIG. 14 depicts GFP expression in Hela cells from purified and unpurified circRNA.

### DETAILED DESCRIPTION OF THE DISCLOSURE

The present disclosure is directed to, inter alia, novel circRNA compositions and methods for RNA affinity purification. In particular, the disclosure relates to circRNA and linear RNA precursor compositions comprising at least one RNA aptamer. The RNA aptamers associated with the disclosed circRNA compositions enable the use of effective affinity purification. Also disclosed herein are methods of making these circRNA-tagged aptamer compositions.

### I. Definitions

Unless otherwise defined herein, scientific and technical terms used in connection with the present invention shall have the meanings that are commonly understood by those of ordinary skill in the art. Exemplary methods and materials are described below, although methods and materials similar or equivalent to those described herein can also be used in the practice or testing of the present invention. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, may provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure. Accordingly, the terms defined immediately below are more fully defined by reference to the specification in its entirety.

The term “approximately” or “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

Depending on context, the term “polynucleotide” or “nucleotide” may encompass a singular nucleic acid as well as plural nucleic acids. In some embodiments, a polynucleotide is an isolated nucleic acid molecule or construct, e.g., circular RNA (circRNA) or plasmid DNA (pDNA). In some embodiments, a polynucleotide comprises a conventional phosphodiester bond. In some embodiments, a polynucleotide comprises a non-conventional bond (e.g., an amide bond, such as found in peptide nucleic acids (PNA)). The term “nucleic acid” may refer to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding a Factor VIII polypeptide contained in a vector is considered isolated for the purposes of the present disclosure. Further examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present disclosure. Isolated polynucleotides or nucleic acids according to the present disclosure further include such molecules produced synthetically. In addition, a polynucleotide or a nucleic acid can include regulatory elements such as promoters, enhancers, ribosome binding sites, or transcription termination signals.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced recombinant technology, but is not necessarily translated from a designated nucleic acid sequence. It can be generated in any manner, including by chemical synthesis.

An “isolated” polypeptide or a fragment, variant, or derivative thereof refers to a polypeptide that is not in its natural milieu. No particular level of purification is required. For example, an isolated polypeptide can simply be removed from its native or natural environment. Recombinantly produced polypeptides and proteins expressed in host cells are considered isolated for the purpose of the disclosure, as are native or recombinant polypeptides which have been separated, fractionated, or partially or substantially purified by any suitable technique.

“Administer” or “administering,” as used herein refers to delivering to a subject a composition described herein, e.g., a chimeric protein. The composition, e.g., the chimeric protein, can be administered to a subject using methods known in the art. In particular, the composition can be administered intravenously, subcutaneously, intramuscularly, intradermally, or via any mucosal surface, e.g., orally, sublingually, buccally, nasally, rectally, vaginally or via pulmonary route. In some embodiments, the administration is intravenous. In some embodiments, the administration is subcutaneous. In some embodiments, the administration is self-administration. In some embodiments, a parent administers the chimeric protein to a child. In some embodiments, the chimeric protein is administered to a subject by a healthcare practitioner such as a medical doctor, a medic, or a nurse.

### II. Circular RNA and Linear Precursor RNA

Disclosed herein are circular RNA (circRNA) compositions comprising a protein coding region and at least one RNA aptamer. Also disclosed herein, are linear precursor RNA compositions comprising a self-splicing ribozyme and protein coding region, wherein the linear precursor RNA comprises at least one RNA aptamer.

As used herein, the term “circular RNA” or “circRNA” refers to an RNA polynucleotide that does not comprise a 5′ end or 3′ end, i.e., a continuous RNA molecule without a 5′ end or 3′ end. Exogenous circRNA constructs containing a protein coding region are previously described and shown to extend the duration of protein expression from full-length RNA. Wesselhoeft et al., (2018), Nat Commun., 9 (1): 2629; Wesselhoeft et al., (2019), Mol Cell., 74 (3): 508-520; WO2019236673.

As used herein, the term “linear RNA precursor” refers to an RNA polynucleotide that is not circular, but that contains sequence motifs to facilitate a circularization reactions, thereby creating a circular RNA. In certain embodiments, the sequence motif that facilitates circularization is a self-splicing ribozyme. The self-splicing ribozyme method orchestrates circularization efficiently in a wide range of RNAs in vitro, including RNAs with a protein coding region. Designing the linear precursor RNA with additional auxiliary sequences aid in creating favorable conditions for splicing (i.e., 5′ external homology arm, 5′ internal homology arm, 5′ spacer sequence, 3′ spacer sequence, 3′ internal homology arm, and 3′ external homology arm). Id. Functional protein was produced exogenous circRNA constructs in eukaryotic cells and translation was successfully initiated by incorporating an internal ribosome entry sites (IRES) and internal polyadenosine tracts.

Exogenous circRNA purified by high performance liquid chromatography displayed exceptional protein production qualities in terms of both quantity of protein produced and stability. However, samples retained impurities and unwanted RNA species including linear precursor RNA, nicked circular RNA, double stranded RNA, triphosphate-RNA, free nucleotides, endotoxins, and solvents.

Provided herein are methods and compositions that facilitate the use of exogenous circRNA for robust and stable protein expression in eukaryotic cells by improving the efficiency, quality, and reliability of circRNA purification methods.

### A. IRES

The translation of circRNAs can only be initiated in a cap-independent fashion because circRNA lacks a 5′ cap and 3′ poly-A tail. IRES-mediated translation of exogenous circRNA is one of the widely accepted mechanisms of circRNA translation initiation. Pamudurti et al., (2017), 66:9-21 e27; Petkovic (2015), Nucleic Acids Res., 43:2454-2465.

In some embodiments, the circRNA disclosed herein comprises an internal ribosome entry site (IRES) which is positioned at the 5′ end of the protein coding region. In some embodiments, the linear precursor RNA disclosed herein comprises an IRES. In some embodiments, the IRES is positioned at the 3′ end of the protein coding region in the linear precursor RNA but shifts to the 5′ end of the protein coding region upon circularization.

In some embodiments, the IRES is derived from Taura syndrome virus, Triatoma virus, Theiler's encephalomyelitis virus, simian Virus 40, Solenopsis invicta virus 1, Rhopalosiphum padi virus, Reticuloendotheliosis virus, fuman poliovirus 1, Plautia stali intestine virus, Kashmir bee virus, Human rhinovirus 2, Homalodisca coagulata virus-1, Human Immunodeficiency Virus type 1, Homalodisca coagulata virus-1, Himetobi P virus, Hepatitis C virus (HCV), Hepatitis A virus, Hepatitis GB virus, Equine rhinitis virus, Ectropis obliqua picorna-like virus, Encephalomyocarditis virus (EMCV), Drosophila C Virus, Crucifer tobamo virus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AMLURUNX1, Drosophila antennapedia, Human AQP4, Human AT1R, Human BAG-1, Human BCL2, Human BiP, Human c-IAP1, Human c-myc, Human elF4G, Mouse NDST4L, Human LEF1, Mouse HIF1 alpha, Human n.myc, Mouse Gtx, Human p27kip1, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Drosophila hairless, S. cerevisiae TFIID, S. cerevisiae YAP1, Human c-src, Human FGF-1, Simian picomavirus, Turnip crinkle virus, an aptamer to eIF4G, Coxsackievirus B3 (CVB3) or Coxsackievirus A (CVB1/2), Dicistroviruses, poliovirus (PV), enterovirus 71 (EV71), human rhinovirus (HRV), foot-and-mouth disease virus (FMDV), or synthetic IRES. In some embodiments, the is derived from a CVB3 IRES. In yet another embodiment, the IRES comprises a polynucleotide sequence of SEQ ID NO: 75. In yet another embodiment, the IRES is encoded by a polynucleotide sequence of SEQ ID NO: 51.

### B. 5′ and 3′ Homology Arms

As used herein, a “homology arm” is any contiguous sequence that is predicted to form base pairs with at least about 75% (e.g., at least about 80%, at least about 85%, at least about 90%, at least about 95%, or 100%) of another homology arm in the RNA (i.e., the circular RNA or linear RNA precursor). A homology arm sequence is about 5 to about 50 nucleotides in length. The homology arm sequence may be located before and adjacent to, or included within, the 3′ intron fragment and/or after and adjacent to, or included within, the 5′ intron fragment. The homology arm sequence is predicted to have less than 50% (e.g., less than 45%, less than 40%, less than 35%, less than 30%, less than 25%) base pairing with unintended sequences in the RNA (e.g., non-homology arm sequences). A “strong homology arm” refers to a homology arm with a Tm of greater than 50° C. when base paired with another homology arm in the RNA.

“Internal homology arms” and “external homology arms” refer to the orientation of the homology arms with respect to the self-splicing PIE fragments and the protein coding region. In the linear precursor RNA, internal homology arms are positioned between the self-splicing PIE fragments and the protein coding region. Upon circularization conditions, the internal homology arms remain in the circular RNA. In the linear precursor RNA, the external homology arms flank the self-splicing PIE fragments. Upon circularization conditions, the external homology arms are excised and are not present in the circular RNA.

In some embodiments, the circRNA disclosed herein comprises a 5′ internal homology arm. In some embodiments, the linear precursor RNA disclosed herein comprises a 5′ internal homology arm. In some embodiments, the 5′ internal homology arm comprises the nucleotide sequence of SEQ ID NO: 70. In some embodiments, the 5′ internal homology arm is about 5 to about 50 nucleotides in length.

In some embodiments, the circRNA disclosed herein comprises a 3′ internal homology arm. In some embodiments, the linear precursor RNA disclosed herein comprises a 3′ internal homology arm. In some embodiments, the 3′ internal homology arm comprises the nucleotide sequence of SEQ ID NO: 71. In some embodiments, the 3′ internal homology arm is about 5 to about 50 nucleotides in length.

In some embodiments, the linear precursor RNA disclosed herein comprises a 5′ external homology arm and a 3′ external homology arm. In some embodiments, the 5′ external homology arm and the 3′ external homology arm comprises the nucleotide sequence of SEQ ID NO: 69 or SEQ ID NO: 72. In some embodiments, the 5′ external homology arm and the 3′ external homology arm are each independently about 5 to about 50 nucleotides in length.

### C. Spacer Sequence

Spacer sequences may be employed to separate different elements in the circular RNA or linear precursor RNA of the disclosure. By separating the different elements, RNA secondary structure may fold better. For example, but in no way limiting, a spacer may be placed at the 5′ end of an IRES to allow the IRES to fold into the proper structure. The spacer sequences can be polyA sequences, polyAC sequences, polyC sequences, polyU sequences, or the spacer sequences can be engineered depending on the spatial constraints of secondary structures that are made by the other elements contained in the linear precursor RNA (e.g., the aptamer, the IRES, and the 5′ and 3′ self-splicing PIE fragments). Spacer sequences may promote circularization by introducing a region of spacer-spacer complementarity to promote the formation of a “splicing bubble” and spacer sequences promote functionality by allowing the highly structured intron portion of the self-splicing PIE fragment and IRES to fold into their correct secondary structures.

In some embodiments, the circular RNA or linear precursor RNA disclosed herein comprises at least one spacer sequence. In some embodiments, the circular RNA or linear precursor RNA comprises two or more spacer sequences. The two or more spacer sequences may comprise identical nucleotide sequences. In other embodiments, at least one of the two or more spacer sequences comprises a distinct nucleotide sequence. In some embodiments, the spacer sequence is about 5 to about 500 nucleotides in length. In some embodiments, the spacer sequence is about 5, about 10, about 15, about 20, about 25, about 30, about 35, about 40, about 45, about 50, about 55, about 60, about 65, about 70, about 75, about 80, about 85, about 90, about 95, about 100, about 150, about 200, about 250, about 300, about 350, about 400, about 450, or about 500 nucleotides in length. In some embodiments, the spacer sequence is longer than about 500 nucleotides in length.

In some embodiments, the circular RNA or linear precursor RNA disclosed herein comprises a 5′ spacer and a 3′ spacer sequence. In some embodiments, the 5′ spacer and the 3′ spacer comprises the nucleotide sequence of SEQ ID NO: 78 or SEQ ID NO: 79.

### D. Self-Splicing Ribozyme Elements and Circularization of the Linear Precursor RNA

The self-splicing ribozyme method of circularization utilizing a permuted group I catalytic intron can circularize long linear precursor RNA and requires only the addition of GTP and Mg2+ as cofactors (i.e., circularization conditions). Petkovic& Muller, (2015) Nucleic Acids Research, 43 (4): 2454-2465. Permuted intron-exon (PIE) splicing strategy consists of fused partial exons flanked by half-intron sequences (i.e., 3′ self-splicing PIE fragment and 5′ self-splicing PIE fragment). Puttaraju & Been, (1992) Nucleic Acids Research, 20 (20): 5357-5364. Upon addition of circularization conditions, linear precursor RNA containing the 3′ and 5′ self-splicing PIE undergo the double transesterification reactions characteristic of group I catalytic introns. During the reactions, the exon elements are fused resulting in the 5′ to 3′ linked circles. Petkovic & Muller, (2015) Nucleic Acids Research, 43 (4): 2454-2465; Wesselhoeft et al., (2018), Nat Commun., 9 (1): 2629.

In some embodiments, the linear precursor RNA disclosed herein comprises at least two catalytic subunits. In some embodiments, the self-splicing ribozyme catalytic subunits derive from either a group I intron or a group II intron RNA transcript or a fragment thereof. In some embodiments, the self-splicing ribozyme catalytic subunits derive from a permuted intron-exon (PIE) sequence from Cyanobacterium anabaena pre-tRNA-Leu gene, T4 phage Td gene, or Tetrahymena pre-rRNA. In some embodiments, RNA catalytic subunits comprise a 3′ self-splicing PIE fragment and a 5′ self-splicing PIE fragment. In some embodiments, the 3′ self-splicing PIE fragment comprises the nucleotide sequence of SEQ ID NO: 73. In some embodiments, the 5′ self-splicing PIE fragment comprises the nucleotide sequence of SEQ ID NO: 74. In some embodiments, the catalytic activity of the two subunits result in a circularized RNA.

In some embodiments, the circRNA disclosed herein comprises a 3′ exon element. In some embodiments, the 3′ exon element comprises the nucleotide sequence of SEQ ID NO: 81. In some embodiments, the circRNA comprising the protein coding region and at least one RNA aptamer comprises a 5′ exon element. In some embodiments, the 5′ exon element comprises the nucleotide sequence of SEQ ID NO: 83.

E. 5′ and 3′ UTR Sequence and polyA Sequences

Previous studies have shown that 5′ and 3′ UTR sequences do not prevent efficient circularization of RNA and can potentially improve the expression of circRNA by acting as additional spacer sequence (See, e.g., WO2019236673). Polyadenylation (polyA) sequences may also function as spacers.

In some embodiments the circRNA disclosed herein contains at least one 5′ untranslated region (5′ UTR), at least one 3′ untranslated region (3′ UTR), and/or at least one polyadenylation (polyA) sequence. In some embodiments, the linear precursor RNA disclosed herein contains at least one 5′ untranslated region (5′ UTR), at least one 3′ untranslated region (3′ UTR), and/or a polyadenylation (polyA) sequence.

In some embodiments, the 5′ UTR comprises the nucleotide sequence of SEQ ID NO: 76. In some embodiments, the 3′ UTR comprises the nucleotide sequence of SEQ ID NO: 77.

In some embodiments, a 5′ UTR may be between about 50 and 500 nucleotides in length. In some embodiments, a 3′ UTR may be between 50 and 500 nucleotides in length or longer. In some embodiments, the circular RNA and linear precursor RNA disclosed herein comprise a 5′ or 3′ UTR that is derived from a gene distinct from the gene encoding the polypeptide in the protein coding region. In some embodiments, the circRNA disclosed herein comprise a 5′ or 3′ UTR that is chimeric. In some embodiments, the linear precursor RNA disclosed herein comprise a 5′ or 3′ UTR that is chimeric.

### F. IVT: Generation of the Linear Precursor

The term “in vitro transcription” or “IVT” relates to a process wherein RNA is synthesized in a cell-free system (in vitro). As disclosed herein, linearized plasmid DNA can be used as template for the generation of linear RNA precursors. The promoter for controlling in vitro transcription can be any promoter for any DNA dependent RNA polymerase. Examples of DNA dependent RNA polymerases are the T7, T3, and SP6 RNA polymerases. A DNA template for in vitro RNA transcription may be obtained by cloning of a nucleic acid, in particular cDNA corresponding to the target RNA to be in vitro transcribed and introducing it into an appropriate DNA for in vitro transcription, for example into plasmid DNA. The cDNA may be obtained by reverse transcription of mRNA, chemical synthesis, or oligonucleotide cloning.

The linear precursor RNA disclosed herein may be synthesized according to any of a variety of known methods. In some embodiments, the linear precursor RNA according to the present invention may be synthesized via in vitro transcription (IVT). Methods for in vitro transcription are known in the art. See, e.g., Geall et al. (2013) Semin. Immunol. 25 (2): 152-159; Brunelle et al. (2013) Methods Enzymol. 530:101-14. Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor. The exact conditions will vary according to the specific application. The presence of these reagents is undesirable in a final RNA product and are considered impurities or contaminants which must be purified to provide a clean and homogeneous linear precursor RNA or resulting circRNA that is suitable for therapeutic use.

G. Total Length and Chemical Modifications to circRNA and Linear Precursor RNA

The methods disclosed herein may be used to purify circRNA or the linear precursor RNA of a variety of nucleotide lengths. In some embodiments, the disclosed methods may be used to purify circRNA or linear precursor RNA of greater than about 1 kb, 1.5 kb, 2 kb, 2.5 kb, 3 kb, 3.5 kb, 4 kb, 4.5 kb, 5 kb, 6 kb, 7 kb, 8 kb, 9 kb, 10 kb, 11 kb, 12 kb, 13 kb, 14 kb, or 15 kb in length. The circRNA or the linear precursor RNA disclosed herein may be modified or unmodified. In some embodiments, the circRNA or the linear precursor RNA disclosed herein contain one or more modifications that typically enhance RNA stability or regulate translation of circRNA. Tang and Lv, (2021), Int J Biol Sci. 17 (9); 2262-2277. Exemplary modifications include backbone modifications, sugar modifications, or base modifications. In some embodiments, the disclosed linear precursor RNA may be synthesized from naturally occurring nucleotides and/or nucleotide analogues (modified nucleotides) including, but not limited to, purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, 3-D-mannosyl-queosine, phosphoramidates, phosphorothioates, peptide nucleotides, methylphosphonates, 7-deazaguanosine, 5-methylcytosine, N6-methyladenosine, and inosine. In some embodiments, the disclosed circRNA or the linear precursor RNA comprise at least one chemical modification including but not limited to, consisting of pseudouridine, N1-methylpseudouridine, 2-thiouridine, 4′-thiouridine, 5-methylcytosine, 2-thio-I-methyl-1-deaza-pseudouridine, 2-thio-I-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-I-methyl-pseudouridine, 4-thio-pseudouridine, 5-aza-uridine, dihydropseudouridine, 5-methyluridine, 5-methyluridine, 5-methoxyuridine, and 2′-O-methyl uridine. In some embodiments, the modified nucleotides comprise N1-methylpseudouridine. The preparation of such analogues is known to a person skilled in the art e.g., from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530, and 5,700,642.

### H. Protein Coding Region

The circRNA or the linear precursor RNA disclosed herein contains a protein coding region encoding for a protein (e.g., a polypeptide or peptide). In some embodiments, the protein coding region is derived from a single gene or a single synthesis or expression construct. However, in some embodiments, the circRNA or the linear precursor RNA compositions disclosed herein comprise multiple protein coding regions and each can or collectively code for one or more proteins.

In some embodiments, the circRNA or the linear precursor RNA comprising the RNA aptamer as disclosed herein encodes a therapeutic polypeptide. In some embodiments, the therapeutic polypeptide comprises an antibody heavy chain, an antibody light chain, an enzyme, or a cytokine.

In some embodiments, the circRNA or the linear precursor RNA encodes a cytokine. Non-limiting examples of cytokines include IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, INF-α, INF-y, GM-CFS, M-CSF, LT-β, TNF-α, growth factors, and hGH.

In one embodiment, the circRNA or the linear precursor RNA comprising the RNA aptamer encodes a genome-editing polypeptide. In some embodiments, the genome-editing polypeptide is a CRISPR protein, a restriction nuclease, a meganuclease, a transcription activator-like effector protein (TALE, including a TALE nuclease, TALEN), or a zinc finger protein (ZF, including a ZF nuclease, ZFN). See, e.g., Int'l Pub. No. WO2020139783.

In some embodiments, the circRNA or the linear precursor RNA encodes an enzyme that is utilized in an enzyme replacement therapy. Examples of enzyme replacement therapy include lysosomal diseases, such as Gaucher disease, Fabry disease, MPS I, MPS II (Hunter syndrome), MPS VI and Glycogen storage disease type II.

In some embodiments, the circRNA or the linear precursor RNA comprising the RNA aptamer encodes an antigen of interest. The antigen may be a polypeptide derived from a virus, for example, influenza virus, coronavirus (e.g., SARS-COV-1, SARS-COV-2, or MERS-related virus), Ebola virus, Dengue virus, human immunodeficiency virus (HIV), hepatitis A virus (HAV), hepatitis B virus (HBV), hepatitis C virus (HCV), herpes simplex virus (HSV), respiratory syncytial virus (RSV), rhinovirus, cytomegalovirus (CMV), zika virus, human papillomavirus (HPV), human metapneumovirus (hMPV), human parainfluenza virus type 3 (PIV3), Epstein-Barr virus (EBV), or chikungunya virus.

The antigen may be derived from a bacterium, for example, Staphylococcus aureus, Moraxella (e.g., Moraxella catarrhalis; causing otitis, respiratory infections, and/or sinusitis), Chlamydia trachomatis (causing chlamydia), Borrelia (e.g., Borrelia burgdorferi causing Lyme Disease), Bacillus anthracis (causing anthrax), Salmonella typhi (causing typhoid fever), Mycobacterium tuberculosis (causing tuberculosis), Propionibacterium acnes (causing acne), or non-typeable Haemophilus influenzae.

Where desired, the circRNA or the linear precursor RNA comprising the RNA aptamer may encode for more than one antigen. In some embodiments, the circRNA or the linear precursor RNA disclosed herein encode for two, three, four, five, six, seven, eight, nine, ten, or more antigens. These antigens can be from the same or different pathogens. For example, a polycistronic protein coding region that can be translated into more than one antigen (e.g., each antigen-coding sequence is separated by a nucleotide linker encoding a self-cleaving peptide such as a 2A peptide) and can be further fused to the aptamer.

In some embodiments, the circRNA or the linear precursor RNA compositions disclosed herein are used in a vaccine. RNA vaccines provide a promising alternative to traditional subunit vaccines, which contain antigenic proteins derived from a pathogen. Vaccines based on RNA allow de novo expression of complex antigens in the vaccinated subject, which in turn allows proper post-translational modification and presentation of the antigens in its natural conformation. Moreover, once established, the manufacturing process for circRNA vaccines can be used for a variety of antigens, enabling rapid development and deployment of circRNA vaccines. A detailed discussion of RNA vaccines can be found in Pardi, et al. (2018) Nat Rev Drug Discov 17, 261-279.

### III. Aptamers

Widespread use of affinity purification of RNA has been limited due to the lack of efficient RNA fusion tags. Unless the RNA to be purified naturally contains a sequence with strong affinity for a target that can be immobilized on the stationary phase (i.e., a chromatography resin), the RNA may require tagging with a specific sequence to do so, analogous to the polyhistidine tag used in protein science.

Disclosed herein are circular RNA compositions which comprise a protein coding region and at least one aptamer. Also disclosed herein are linear precursor RNA compositions which comprise at least a self-splicing ribozyme and protein coding region, wherein the linear precursor RNA comprises at least one RNA aptamer. The aptamers associated with these circular RNA and linear precursor RNA compositions enable the use of affinity purification with minimal impact on translation efficiency and immunogenicity. Also disclosed herein are methods of making such circular RNA- and linear precursor RNA-tagged aptamer compositions.

The term “aptamer” as used herein refers to any nucleic acid sequence that has a non-covalent binding site for a specific target. Exemplary aptamer targets include nucleic acid sequence, protein, peptide, antibody, small molecule, mineral, antibiotic, and others. The aptamer binding site may result from secondary, tertiary, or quaternary conformational structure of the aptamer.

The term “RNA aptamer” as used herein refers to an aptamer comprised of RNA. In some embodiments, the RNA aptamer is included in the nucleotide sequence of the circRNA or the linear precursor RNA. In other embodiments, the RNA aptamer is separate from the nucleotide sequence of the circRNA or the linear precursor RNA.

Aptamers are typically capable of binding to specific targets with high affinity and specificity. Aptamers have several advantages over other binding proteins (e.g., antibodies). For example, aptamers can be engineered completely in vitro (e.g., via a SELEX aptamer selection method), can be produced by chemical synthesis, possess desirable storage properties, and elicit little or no immunogenicity in therapeutic applications. See, generally, Proske et al., (2005) Appl. Microbiol. Biotechnol 69:367-374.

Aptamers have historically been used to modulate gene expression by directly binding to ligands. These aptamers act similarly to regulatory proteins, forming highly specific binding pockets for the target, followed by conformational changes.

In some embodiments, the RNA aptamer is synthetically derived. In some embodiments, the RNA aptamer is naturally derived from prokaryotes and/or eukaryotes. In some embodiments, the RNA aptamer is derived from a hairpin RNA, a tRNA, or a riboswitch.

In some embodiments the RNA aptamer is derived from a riboswitch. Riboswitches are regulatory RNA elements that act as small molecule sensors to control gene transcription and translation. Several riboswitch classes are known in the art. Exemplary riboswitches include B12 riboswitch, TPP riboswitch, SAM riboswitch, guanine riboswitch, FMN riboswitch, lysine riboswitch, and the PreQ1 riboswitch.

In some embodiments, the RNA aptamer is a split aptamer. Split aptamers are analogs to split-protein systems (e.g., beta-galactosidase) and rely on two or more short nucleic acid strands that assemble into a higher order structure upon the presence of a specific target. Debais et al. (2020) Nucleic Acids Res 48 (7): 3400-3422. An exemplary split aptamer is the ATP-aptamer. Sassanfar & Szostak (1993) Nature 364 (6437)-550-553. The ATP aptamer is an RNA aptamer that was divided into two RNA fragments by removing the loop that closes the stem and by extending each fragment with additional nucleotides to compensate for the loss of stability. Neither of the two RNA fragments bind ATP alone but in the presence of ATP the binding ability is reactivated. Debiais et al. (2020) Nucleic Acids Res 48 (7): 3400-3422.

In other embodiments, the split aptamer is reformed through the circularization of a linear precursor RNA. In this context, the split aptamer comprises a 5′ portion and a 3′ portion. Each portion may be of any length that is less than the full, un-split aptamer. The 5′ portion and 3′ portion together form the full un-split aptamer. For linear precursor RNA that comprise a 3′ exon element and a 5′ exon element, then the 5′ portion of the split aptamer is positioned 3′ of the 5″ exon element and the 3′ portion of the split aptamer is positioned 5′ of the 3″ exon element. For linear precursor RNA that do not comprise a 5′ exon element and a 3′ exon element, then the 5′ portion of the split aptamer is positioned 3′ of the 3′ internal homology arm and the 3′ portion of the split aptamer is positioned 5′ of the 5′ internal homology arm.

In certain embodiments, the split aptamer is reformed to a functional aptamer upon circularization of the linear precursor RNA.

In some embodiments, the RNA aptamer is an X-aptamer. X-aptamers are engineered with a combination of natural and chemically-modified nucleotides to improve binding affinity, specificty, and versatility. An exemplary embodiment of a X-aptamer is the PS2-aptamer. The PS2-aptamer is an RNA aptamer that contains a phosphorodithioate (i.e., PS2) substitution at a single nucleotide of RNA aptamer which increases the aptamer's binding affinity from a nanomolar to a picomolar range. Abeydeera et al. (2016) Nucleic Acids Res. 44 (17): 8052-8064.

In some embodiments, the RNA aptamer binds to a ligand. In some embodiments the ligand is utilized in an affinity purification system. In some embodiments, the affinity ligand comprises protein A, protein G, streptavidin, glutathione (GSH), dextran (sephadex), cellulose (e.g., diethylaminoethyl cellulose) or a fluorescent molecule. In some embodiments, the affinity ligand is immobilized on a chromatography resin.

In some embodiments, the affinity ligand comprises protein A. DNA aptamers have been shown previously to target protein A. See, e.g., Stoltenburg et al. (2016) Sci Rep. 6:33812.

In some embodiments, the disclosed RNA aptamers bind streptavidin. Streptavidin-binding aptamers are described in, e.g., Srisawat & Engelke (2001) RNA 7 (4): 632-641. An exemplary RNA aptamer that binds streptavidin is S1. In some embodiments, the RNA aptamer comprises the nucleotide sequence of UCAUGCAAGUGCGUAAGAUAGUCGCGGGCCGGGGGCGUAU (SEQ ID NO: 90).

Also disclosed herein are RNA aptamers that bind to sephadex. Sephadex-binding aptamers are described in, e.g., Srisawat et al. (2001) Nucleic Acid Res 29 (2): e4. An exemplary RNA aptamer that binds sephadex (e.g., Sephadex G-100) is Sephadex D8. In some embodiments, the RNA aptamer comprises the nucleotide sequence of GUCCGAGUAAUUUACGUUUUGAUACGGUUGCGGAACUUGC (SEQ ID NO: 91).

Also disclosed herein are RNA aptamers that bind to glutathione (GSH). Glutathione-binding aptamers are described in, e.g., Bala, et al. (2011). RNA Biology 8 (1): 101-111. In some embodiments, the RNA aptamer is GSHapt 8.17 or GSHapt 5.39.

Also disclosed herein are RNA aptamers that bind to 6×His. 6×His corresponds to amino acid sequence of 6 consecutive histidine residues. The 6×His sequence may be isolated and optionally immobilized on a chromatography resin. Alternatively, the 6×His sequence may be present as a N or C-terminal tag on a polypeptide, optionally wherein the 6×His-tagged polypeptide is immobilized on a chromatography resin. 6×His-binding aptamers are described in, e.g., Tsuji, et al. (2009). Biochem Biophys Res Commun. 386 (1): 227-231. In some embodiments, the RNA aptamer is shot47 or 47s. In some embodiments, the RNA aptamer comprises the nucleotide sequence of GGGUACGCUCAGGUAUAUUGGCGCCUUCGUGGAAUGUCAGUGCCUGGACGUGCAGU (SEQ ID NO: 84). In some embodiments, the RNA aptamer comprises the nucleotide sequence of GGGACGCUCACGUACGCUCACGUCCGAUCGAUACUGGUAUAUUGGCGCCUUCGUGGAAUG UCAGUGCCUGGACGUGCAGU (SEQ ID NO: 85). In some embodiments, the RNA aptamer comprises the nucleotide sequence of GGGUAUAUUGGCGCCUUCGUGGAAUGUCAGUGCCUGG (SEQ ID NO: 86).

Also disclosed herein are RNA aptamers that bind to a MS2 coat protein (MCP). In some embodiments, the RNA aptamer comprises the nucleotide sequence of GGCCAACAUGAGGAUCACCCAUGUCUGCAGGGCC (SEQ ID NO: 87). In some embodiments, the RNA aptamer comprises the nucleotide sequence of ACAUGAGGAUCACCCAUG (SEQ ID NO: 88). In some embodiments, the RNA aptamer comprises the nucleotide sequence of ACAUGAGGAUCACCCAUGU (SEQ ID NO: 89). In some embodiments, the aptamer-containing circular RNA or linear RNA precursor described herein binds to an MCP immobilized on a chromatography resin. M2 aptamers are described in further detail in Bertrand et al. (1998). Molecular cell, 2 (4), 437-445.

Also disclosed herein are RNA aptamers that bind to a fluorescent molecule. Examples of such aptamers are described in, e.g., Paige et al. (2011) Science 333 (6042): 642-646. In some embodiments, the RNA comprises the aptamer nucleotide sequence of GAAGGGACGGUGCGGAGAGGAGA (SEQ ID NO: 92). The recited RNA aptamer is designated RNA Mango and binds the fluorescent molecule Thizole Orange (TO), such as TO1-biotin as described in Dolgosheina et al. (2014) ACS Chemical Biology, 9 (10): 2412-2420.

In some embodiments, the RNA aptamer comprises the nucleotide sequence of AGCUUAUCCAUUGCAUCUCGGAUGAGCU (SEQ ID NO: 93). The recited RNA aptamer is designated U1 hp and binds the spliceosomal protein U1A as described in Katsamba et al. (2001) J Biol Chem. 276 (24): 21476-81.

In some embodiments, the RNA aptamer comprises a S1m aptamer or a derivative or fragment thereof. In some embodiments, the S1m aptamer used according to the instant disclosure is the aptamer described in Bachler et al. (1999) RNA 5 (11): 1509-1516, Srisawat & Engelke (2001) RNA 7 (4): 632-641, or Li & Altman. (2002) Nuc. Acids Res. 30 (17): 3706-3711. In some embodiments, the RNA aptamer comprises the nucleotide sequence of SEQ ID NO: 65 or SEQ ID NO: 66. In some embodiments, the RNA adapter is encoded by the nucleotide sequence of SEQ ID NO: 52 or SEQ ID NO: 53.

In some embodiments, the RNA aptamer comprises a Sm aptamer.

In some embodiments, the RNA aptamer is about 30-200 nucleotides in length. In some embodiments, the RNA aptamer is about 50-200 nucleotides in length. In some embodiments, the RNA aptamer is about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, or about 200 nucleotides in length.

In some embodiments, the aptamer (e.g., RNA aptamer) is not a histone stem-loop. As used herein, the term “histone stem-loop” refers to a stem-loop RNA structure that is typically found in histone-encoding mRNA. The histone stem-loop binds the stem-loop binding protein (SLBP) and is used to regulate histone expression during the cell cycle. Histone stem-loops are described in further detail in Lopez et al. (RNA. 14 (1): 1-10. 2008) and WO2013120498.

In some embodiments, the aptamer (e.g., RNA aptamer) is not an internal ribosome entry site (IRES). In some embodiments, the aptamer (e.g., RNA aptamer) does not bind a ribosome or a protein that regulates protein translation. In some embodiments, the aptamer (e.g., RNA aptamer) does not bind the protein elF4G. In some embodiments, the aptamer (e.g., RNA aptamer) is capable of binding a specific target (e.g., a protein) immobilized on a surface (e.g., a protein immobilized on a surface, such as a crosslinked agarose or crosslinked dextran).

### A. Aptamer Location

Disclosed herein are RNA aptamers which include aptamers at various locations with respect to the other elements present in the linear precursor RNA or the subsequent circRNA. Selection of location of the RNA aptamer on the circRNA or the linear precursor RNA can be evaluated with respect to both the magnitude of regulation of translation and basal expression level.

In some embodiments, the RNA aptamer in the circRNA is positioned: a) before the 3′ exon element, b) between the 3′ exon element and the 5′ internal homology arm, c) between the 5′ internal homology arm and the 5′ spacer sequence, d) between the 5′ spacer sequence and the IRES, e) between the protein coding region and the 3′ spacer sequence, f) between the 3′ spacer sequence and the 3′ internal homology arm, g) between the 3′ internal homology arm and the 5′ exon element, h) after the 5′ exon element, j) between the 3′ exon and the IRES, and/or i) between the IRES and the 5′ exon element.

In some embodiments, the RNA aptamer in the circRNA is positioned: a) before the 3′ exon element, b) between the 3′ exon element and the 5′ internal homology arm, c) between the 5′ internal homology arm and the 5′ spacer sequence, d) between the 5′ spacer sequence and the protein coding region, e) between the IRES and the 3′ spacer sequence, f) between the 3′ spacer sequence and the 3′ internal homology arm, g) between the 3′ internal homology arm and the 5′ exon element, h) after the 5′ exon element, i) between the 3′ exon and the protein coding region, and/or j) between the protein coding region and the 5′ exon element.

In some embodiments, the RNA aptamer in the linear precursor RNA is positioned: a) before the 5′ external homology arm, b) between the 5′ external homology arm and the 3′ self-splicing PIE fragment, c) between the 3′ self-splicing PIE fragment and the 5′ internal homology arm, d) between the 5′ internal homology arm and the 5′ spacer sequence, e) between the 5′ space sequence and the IRES, f) after the protein coding region but before the 3′ spacer sequence, g) between the 3′ spacer sequence and the 3′ internal homology arm, h) between the 3′ internal homology arm and the 5′ self-splicing PIE fragment, i) between the 5′ self-splicing PIE fragment and the 3′ external homology arm, and/or j) after the 3′ external homology arm.

In some embodiments, the RNA aptamer in the linear precursor RNA is positioned: a) before the 5′ external homology arm, b) between the 5′ external homology arm and the 3′ self-splicing PIE fragment, c) between the 3′ self-splicing PIE fragment and the 5′ internal homology arm, d) between the 5′ internal homology arm and the 5′ spacer sequence, e) between the 5′ space sequence and the protein coding region, f) after the IRES but before the 3′ spacer sequence, g) between the 3′ spacer sequence and the 3′ internal homology arm, h) between the 3′ internal homology arm and the 5′ self-splicing PIE fragment, i) between the 5′ self-splicing PIE fragment and the 3′ external homology arm, and/or j) after the 3′ external homology arm.

In some embodiments, the RNA aptamer does not have to be bound directly to the circRNA or the linear precursor RNA. In some embodiments, the RNA aptamer is attached to a linker. See, e.g., Elenko et al. (2009) J Am Chem Soc. 131 (29): 9866-9867.

In some embodiments, the RNA aptamer can be removed from the circRNA or the linear precursor RNA after affinity purification. This may be achieved, for example, using DNA oligonucleotides which hybridize to the RNA aptamer or RNA scaffold. The resulting duplex can then be cleaved with an enzyme such as RNase H. See, e.g, Batey RT. (2014). Curr Opin Struct Biol. 26:1-8.

### B. Aptamer Copy Number

An increase in aptamer copy number may allow aptamers to create a larger three-dimensional structure (i.e., enhancing the number of affinity ligand binding sites available or creating a unique ligand binding site). A strategic arrangement of aptamer copies may allow for increased avidity with the cognate affinity ligand.

In some embodiments, the circRNA or the linear precursor RNA used in the disclosed methods and compositions comprises multiple copies of an aptamer. Previous reports have shown that using a single small-molecule binding aptamer in the 5′-UTR enables 8-fold repression of translation upon ligand addition, but using three aptamers causes a 37-fold repression. Kotter et al., (2009). Nucleic Acids Res. 37 (18): e120. In some embodiments, the copy number of aptamers introduced into the circRNA or the linear precursor RNA is one, two, three, four, five, six, seven, eight, nine, ten, or more.

In some embodiments, the RNA aptamer comprises multiple copies of an aptamer sequence. In some embodiments, the RNA aptamer comprises the nucleotide sequence of SEQ ID NO: 65.

In some embodiments, copies of the aptamer are in repeat tandem configuration. The 4XS1m aptamer disclosed herein is an example of a multiple copy aptamer in a repeat tandem configuration.

### IV. RNA Scaffolds

In some embodiments, the circular RNA and linear RNA precursor compositions disclosed herein comprise an RNA aptamer that is embedded in an RNA scaffold. As used herein, the term “RNA scaffold” refers to a noncoding RNA molecule that can assemble to have a predefined structure which creates spatial architecture to organize, protect, or enhance the properties of a functional module of interest. Exemplary functional modules can be nucleic acids (e.g., aptamers) or protein. In some embodiments, the RNA scaffolds suitable for use according to the instant disclosure can be associated with an RNA without disrupting the RNA structure. Furthermore, suitable RNA scaffolds allow for an RNA aptamer to be embedded without disrupting the RNA structure. In some embodiments, the RNA scaffolds used according to the instant disclosure can be any RNA scaffolds which do not have a significant negative impact on RNA expression or translation.

An RNA scaffold's predefined structure contains RNA-specific sequence motifs for self-assembly such as base-pairing between hairpin stems (kissing loops) and/or chemical modifications, Myhrvold & Silver (2015) Nat Struct Mol Bio 22 (1): 8-10. RNA-specific sequence motifs can form secondary (i.e., two-dimensional) and/or tertiary (i.e., three-dimensional) structures. In some embodiments, the RNA scaffold comprises at least one secondary structure motif. In some embodiments, the RNA scaffold comprises at least one tertiary structure motif. Common secondary and/or tertiary RNA structural motifs include open and stacked three-way junctions, four-way junctions, four-way junctions similar to Holliday's structures, stem-loops (i.e., hairpin loops), interior loops (i.e., internal loops), bulges, tetraloops, multibranch loops, pseudoknots and knots, 90° kinks, and pseudo-torsional angles. Shanna et al. (2021) Molecules 26 (5): 1422.

RNA scaffolds can either be derived from nature (e.g., attenuators, tRNA, riboswitches, terminators) or artificially engineered to form secondary or tertiary RNA structure. Delebecque et al. (2012) Nat Protoc 7 (10): 1797-1807. Typically, in order to retain the RNA scaffold predefined structure, the RNA scaffold's RNA loop(s) (e.g., a hairpin loop) are the target regions for embedding the functional module of interest. See, e.g., US20050282190 A1. The RNA scaffold's predefined structure can be modified, however, to have additional desirable properties. For example, the predefined RNA scaffold structure may be modified to become resistant to one or both of exonuclease digestion and endonuclease digestion.

In some embodiments, the circular RNA or linear precursor RNA compositions disclosed herein comprise an RNA aptamer that is embedded in a transfer RNA (tRNA). Transfer RNA (tRNA) scaffolds are an attractive tagging candidate in affinity purification systems, as tRNAs fold into canonical, stable clover-leaf structures that are resistant to unfolding and can protect RNA fusions from nuclease degradation. It has been demonstrated that embedding an aptamer in the anticodon loop of a tRNA scaffold promotes proper folding. See generally, Ponchon and Dardel (2007) Nat. Methods 4 (7): 571-576; Ponchon et al. (2013) Nucleic Acids Res. 41: e150. Use of an RNA aptamer embedded in a tRNA scaffold has been demonstrated to successfully pull down transcript-specific RNA-binding proteins from cell lysates. Iioka H et al. (2011) Nuc. Acids Res. 39 (8): e53.

In some embodiments, the circRNA or the linear precursor RNA compositions disclosed herein comprise an RNA aptamer that is embedded in a tRNA which comprises the nucleotide sequence of SEQ ID NO: 67.

In some embodiments, the RNA aptamer is embedded in a tRNA hairpin loop of the tRNA. In some embodiments, the RNA aptamer is embedded in a tRNA anticodon loop. In some embodiments, the RNA aptamer is embedded in a tRNA D loop. In some embodiments, the RNA aptamer is embedded in a tRNA T loop.

Other exemplary RNA scaffolds include ribosomal RNA (rRNA) and ribozymes. In some embodiments, the RNA aptamer is embedded in a ribosomal RNA. In some embodiments, the RNA aptamer is embedded in a ribozyme. In some embodiments, the ribozyme is catalytically inactive.

### V. Affinity Purification of RNA

In one aspect, disclosed herein are methods for purifying a circular RNA sample.

In some embodiments, the disclosed method for purifying circular RNA, comprises the steps of: (a) contacting a sample comprising the circular RNA disclosed herein with an affinity ligand that is immobilized on a chromatography resin, wherein the RNA aptamer comprises binding affinity for the affinity ligand; (b) eluting the circular RNA from the chromatography resin; and (c) purifying the circular RNA from the sample.

In some embodiments, the disclosed method for purifying a linear precursor RNA, comprises the steps of: (a) contacting a sample comprising the linear precursor RNA disclosed herein with an affinity ligand that is immobilized on a chromatography resin, wherein the RNA aptamer comprises binding affinity for the affinity ligand; (b) eluting the linear precursor RNA from the chromatography resin; and (c) purifying the linear precursor RNA from the sample.

In some embodiments, the disclosed methods comprise one or more washing steps between the contacting step (a) and the eluting step (b).

In some embodiments, the disclosed method for purifying a circular RNA, comprising the steps of: (a) contacting a sample comprising the circular RNA with an affinity ligand that is immobilized on a chromatography resin; (b) eluting the circular RNA from the chromatography resin; and (c) isolating the circular RNA from the sample, wherein the circular RNA comprises a protein coding region and at least one RNA aptamer, wherein the RNA aptamer comprises binding affinity for the affinity ligand.

In some embodiments, the disclosed method for purifying a linear precursor RNA, comprising the steps of: (a) contacting a sample comprising the linear precursor RNA with an affinity ligand that is immobilized on a chromatography resin; (b) eluting the linear precursor RNA from the chromatography resin; and (c) isolating the linear precursor RNA from the sample, wherein the linear precursor RNA comprises a protein coding region and at least one RNA aptamer, wherein the RNA aptamer comprises binding affinity for the affinity ligand.

In some embodiments, the disclosed methods result in circular RNA or linear precursor RNA that is greater than or equal to 90% pure. In some embodiments, the disclosed methods result in circular RNA and nicked circular RNA that is greater than or equal to 90% pure.

Affinity chromatography is one purification method that can be used with the circRNA or the linear precursor RNA compositions and methods disclosed herein. The RNA aptamers disclosed herein comprise binding affinity for the selected affinity ligand. The selected affinity ligand is immobilized (e.g., crosslinked) on a chromatography resin. The circRNA or the linear precursor RNA comprising the RNA aptamer therefore binds with the resin containing the affinity ligand. The chromatography resin material is preferably present in a column, wherein the sample containing RNA is loaded on the top of the column and the eluent is collected at the bottom of the column.

The chromatography resin can be any material that is known to be used as a stationary phase in chromatography methods. The type of molecules used as affinity ligands, which interact with the RNA aptamers disclosed herein, can be a variety of types. Non-exhaustive examples of affinity ligands are antibodies, proteins, oligonucleotides, dyes, boronate groups, or chelated metal ions. The stationary phase may be composed of organic and/or inorganic material.

The most widely used stationary phase materials are hydrophilic carbohydrates such as cross-linked agarose and synthetic copolymer materials. These materials may comprise derivatives of cellulose, polystyrene, synthetic poly amino acids, synthetic polyacrylamide gels, or a glass surface. Further examples of materials that can be used as chromotagraphy resins are polystyrenedivinylbenzenes, silica gel, silica gel modified with non-polar residues, or other materials suitable for gel chromatograpy or other chromatographic methods, such as dextran, sephadex, agarose, dextran/agarose mixtures, and others known in the art.

The chromotography resin can be functionalized with affinity ligands for which the RNA aptamer has binding affinity. In some embodiments, the resin may be an agarose media or a membrane functionalized with phenyl groups (e.g., Phenyl Sepharose™ from GE Healthcare or a Phenyl Membrane from Sartorius), Tosoh Hexyl, CaptoPhenyl, Phenyl Sepharose™ 6 Fast Flow with low or high substitution, Phenyl Sepharose™ High Performance, Octyl Sepharose™ High Performance (GE Healthcare); Fractogel™ EMD Propyl or Fractogel™ EMD Phenyl (E. Merck, Germany); Macro-Prep™ Methyl or Macro-Prep™ t-Butyl columns (Bio-Rad, California); WP HI-Propyl (C3)™ (J. T. Baker, New Jersey) or Toyopearl™ ether, phenyl or butyl (TosoHaas, PA). ToyoScreen PPG, ToyoScreen Phenyl, ToyoScreen Butyl, and ToyoScreen Hexyl are based on rigid methacrylic polymer beads. GE HiScreen Butyl FF and HiScreen Octyl FF are based on high flow agarose based beads. Preferred are Toyopearl Ether-650M, Toyopearl Phenyl-650M, Toyopearl Butyl-650M, Toyopearl Hexyl-650C (TosoHaas, PA), POROS-OH (ThermoFisher) or methacrylate based monolithic columns such as CIM-OH, CIM-SO3, CIM-C4 A and CIM C4 HDL which comprise OH, sulfate or butyl ligands, respectively (BIA Separations).

In some embodiments, the chromatography resin comprises protein A as an affinity ligand. Exemplary protein A resins include Byzen Pro Protein A resin (MilliporeSigma; 18887), Dynabeads Protein A Magnetic Beads (ThermoFisher; 10001D), Pierce Protein A Agarose (ThermoFisher; 20334), Pierce Protein A/G Plus Agarose (ThermoFisher; 20423), Pierce Protein A Plus UltraLink (ThermoFisher; 53142), Pierce Recombinant Protein A Agarose (ThermoFisher), POROS MabCapture A Select (ThermoFisher).

In some embodiments, the chromatography resin comprises streptavidin as an affinity ligand. Exemplary stretavidin resins include Streptavidin-Agarose from Streptomyces avidinii (MilliporeSigma; S1638), Pierce Steptavidin Plus UltaLink Resin (ThermoFisher; 53117), Pierce High Capacity Steptavisin Agarose (ThermoFisher; 20357), Streptavidin 6HC Agarose Resin (ABT; STV6HC-5), Streptavidin Resin—Amintra (Abcam; ab270530).

In some embodiments, the chromatography resin comprises glutathione (GSH) as an affinity ligand. Exemplary GSH resins include Glutathione Resin (GenScript; L00206), Pierce Glutathione Agarose (ThermoFisher; 16102BID), Glutathione Sepharose 4B GST-tagged Protein Resin 9Cytiva; 17075605); Glutathione Affinity Resin-Amintra (Abcam; ab270237).

### VI. Vectors

In one aspect, disclosed herein are vectors comprising the linear precursor RNA disclosed herein. The nucleic acid sequences encoding a protein of interest (e.g., the protein coding region encoding a therapeutic polypeptide) can be cloned into a number of types of vectors. For example, the nucleic acids can be cloned into a vector including, but not limited to a plasmid, a phagemid, a phage derivative, an animal virus, and a cosmid. Vectors of particular interest include expression vectors, replication vectors, probe generation vectors, sequencing vectors and vectors optimized for in vitro transcription.

In one embodiment, the vector is used to express the linear precursor RNA in a host cell. In another embodiment, the vector is used as a template for IVT. The construction of optimally translated IVT RNA suitable for therapeutic use is disclosed in detail in Sahin, et al. (2014). Nat. Rev. Drug Discov. 13, 759-780; Weissman (2015). Expert Rev. Vaccines 14, 265-281.

In some embodiments, the vectors disclosed herein comprise the following, from 5′ to 3′: a) a 5′ external homology arm, b) a 5′ self-splicing PIE fragment, c) a 5′ internal homology arm, d) a 5′ spacer sequence, e) an internal ribosome entry site (IRES), f) a protein coding region, g) a 3′ spacer sequence, h) a 3′ internal homology arm, i) a 3′ self-splicing PIE fragment, and j) a 3′ external homology arm, wherein the RNA aptamer is present at one or both of the 5′ end or 3′ end of any one of elements a)-j).

In some embodiments, the vectors disclosed herein also comprise a polynucleotide sequence 5′ UTR, a polynucleotide sequence 3′ UTR, a polynucleotide sequence encoding a polyA sequence and/or a polyadenylation signal.

A variety of RNA polymerase promoters are known in the art. In one embodiment, the promoter is a T7 RNA polymerase promoter. Other useful promoters include, but are not limited to, T3 and SP6 RNA polymerase promoters. Consensus nucleotide sequences for T7, T3 and SP6 promoters are known in the art.

Also disclosed herein are host cells (e.g., mammalian cells, e.g., human cells) comprising the vectors or RNA compositions disclosed herein.

Polynucleotides can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, biolistic particle delivery systems such as “gene guns” (see, for example, Nishikawa, et al. (2001). Hum Gene Ther. 12 (8): 861-70, or the TransIT-RNA transfection Kit (Mirus, Madison WI).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present invention, in order to confirm the presence of the circRNA or the linear precursor RNA sequence in the host cell, a variety of assays may be performed. Such assays are well known to those of skill in the art.

### VII. Pharmaceutical Compositions

RNA purified according to this invention is useful as a component in pharmaceutical compositions, for example for use as a vaccine. These compositions will typically include RNA and a pharmaceutically acceptable carrier. A pharmaceutical composition of the invention can also include one or more additional components such as small molecule immunopotentiators (e.g., TLR agonists). A pharmaceutical composition of the invention can also include a delivery system for the RNA, such as a liposome, an oil-in-water emulsion, or a microparticle. In some embodiments, the pharmaceutical composition comprises a lipid nanoparticle (LNP). In one embodiment, the composition comprises an antigen-encoding nucleic acid molecule encapsulated within a LNP. In some embodiments, the LNP comprises at least one cationic lipid. In some embodiments, the LNP comprises a cationic lipid, a polyethylene glycol (PEG) conjugated (PEGylated) lipid, a cholesterol-based lipid, and a helper lipid.

In order that this invention may be better understood, the following examples are set forth. These examples are for purposes of illustration only and are not to be construed as limiting the scope of the invention in any manner.

### EXAMPLES

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

### Example 1: Design of Aptamer-Tagged Circular RNA

Previous studies had demonstrated that aptamer tagged mRNA could be useful for the purification of linear RNA species. See WO2023031856A1, incorporated herein by reference in its entirety.

As described herein, the following example discloses the design of aptamer tagged circular RNA (circRNA) or the aptamer tagged linear precursor RNA, which is used to generate the circRNA.

The work described below utilized the S1m aptamer or a tRNA-S1m aptamer, each capable of binding streptavidin. The DNA nucleotide sequence encoding for the S1m aptamer and the tRNA-S1m aptamer are shown below.

SEQ ID NO: 52_S1m aptamer Tag (60 bp)
ATGCGGCCGCCGACCAGAATCATGCAAGTGCGTAAGATAGTCGCGGGTC
GGCGGCCGCAT
SEQ ID NO: 54_tRNA-S1m aptamer Tag (134 bp)
GCCCGGATAGCTCAGTCGGTAGAGCAGCGGCCTATGCGGCCGCCGACCA
GAATCATGCAAGTGCGTAAGATAGTCGCGGGTCGGCGGCCGCATTCGAG
GCCGCGTCCAGGGTTCAAGTCCCTGTTCGGGCGCCA

The S1m aptamer and the tRNA-S1m aptamer sequence present in the circular RNA and/or linear precursor RNA are shown below:

| | |
|---|---|
| SEQ ID NO. | AUGCGGCCGCCGACCAGAAUCAUGCAAGUGCGUAAGAU |
| 65: | AGUCGCGGGUCGGCGGCCGCAU |
| S1m aptamer | |
| SEQ ID NO. | AUGCGGCCGCCGACCAGAAUCAUGCAAGUGCGUAAGAU |
| 66: | AGUCGCGGGUCGGCGGCCGCAUCUGCUGGGAAGCUACG |
| 4xS1m | AUCCGUAGAAAAUGCGGCCGCCGACCAGAAUCAUGCAA |
| aptamer | GUGCGUAAGAUAGUCGCGGGUCGGCGGCCGCAUCUGCU |
| | GGGUAGCUGUGAACCGUAGAAAAUGCGGCCGCCGACCA |
| | GAAUCAUGCAAGUGCGUAAGAUAGUCGCGGGUCGGCGG |
| | CCGCAUCUGCUGGGAAGCUACGAUCCGUAGAAAAUGCG |
| | GCCGCCGACCAGAAUCAUGCAAGUGCGUAAGAUAGUCG |
| | CGGGUCGGCGGCCGCAU |
| SEQ ID NO. | GCCCGGAUAGCUCAGUCGGUAGAGCAGCGGCCUAUGCG |
| 67: | GCCGCCGACCAGAAUCAUGCAAGUGCGUAAGAUAGUCG |
| tRNA-S1m | CGGGUCGGCGGCCGCAUUCGAGGCCGCGUCCAGGGUUC |
| aptamer | AAGUCCCUGUUCGGGCGCCA |

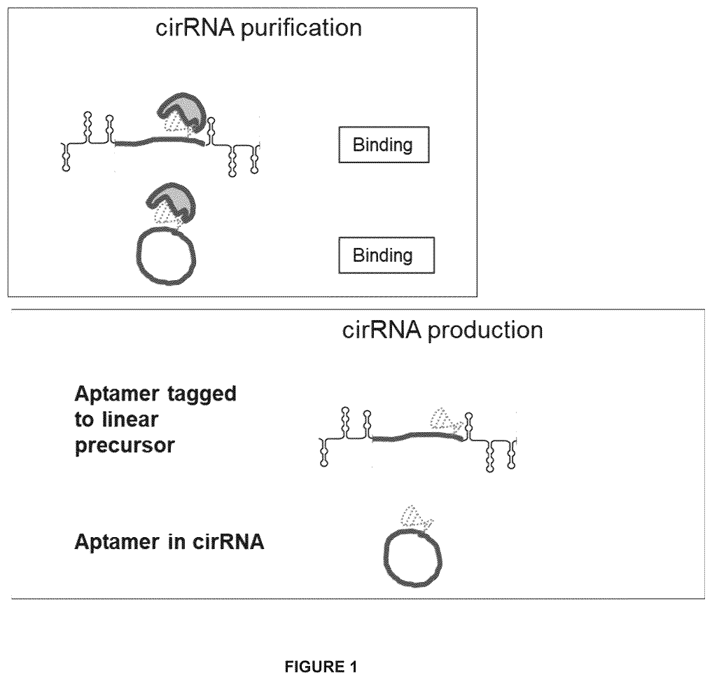

FIG. 1 depicts the experimental schematic of aptamer tagged linear precursor or aptamer tagged circRNA that were tested in streptavidin Sepharose bead affinity purification. The left panel shows the orientation of the aptamer tagged linear precursor RNA with respect to the flanking Anabaena PIE sequence. Anabaena PIE sequence reacted under group I intron splicing conditions resulting in synthesis of the aptamer tagged circRNA. The right panel shows that the presence of the intact aptamer in either the linear precursor RNA or the circRNA species enabled binding to the affinity matrix during purification.

To initially obtain the linear precursor RNA and subsequent circRNA, DNA plasmids were designed.

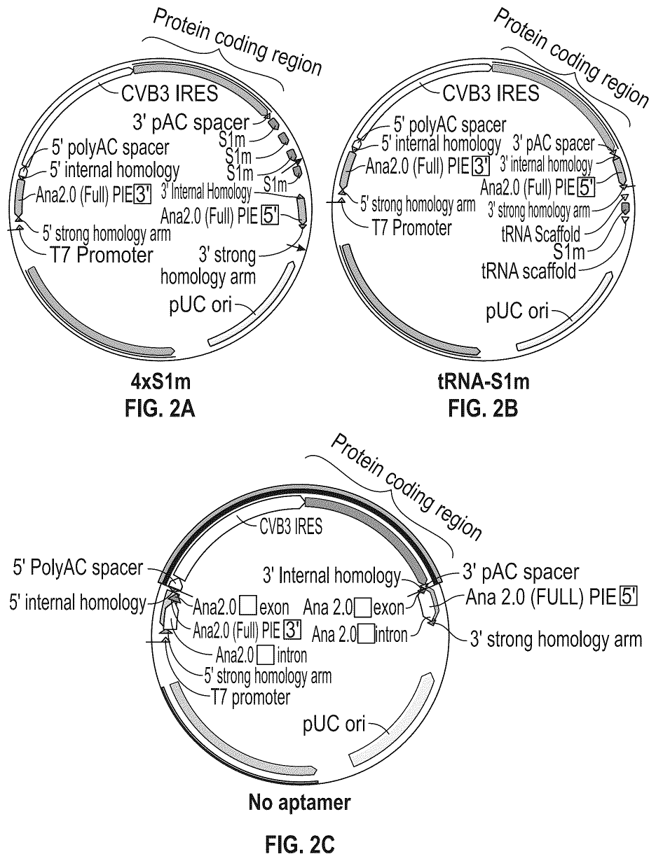

FIG. 2A depicts the plasmid map encoding the 4xS1m aptamer, the linear precursor RNA, and the Anabaena PIE sequences used for RNA circularization. The plasmid elements are arranged in the following 5′ to 3′ order: a T7 promoter, a 5′ external homology arm, a 3′ Anabaena intron/exon fragment, a 5′ internal homology arm, a 5′ polyAC spacer, a CVB3 IRES, a protein coding region, a 3′ polyAC spacer, a 4xS1m aptamer, a 3′ internal homology arm, a 5′ Anabaena intron/exon fragment, and a 3′ external homology arm.

FIG. 2B depicts the plasmid map encoding the tRNA-S1m aptamer, the linear precursor RNA, and the Anabaena PIE sequences used for RNA circularization. The plasmid elements are arranged in the following 5′ to 3′ order: a T7 promoter, a 5′ external homology arm, a 3′ Anabaena intron/exon fragment, a 5′ internal homology arm, a 5′ polyAC spacer, a CVB3 IRES, a protein coding region, a 3′ polyAC spacer, a 3′ internal homology arm, a 5′ Anabaena intron/exon fragment, a 3′ external homology arm, and a tRNA-S1m aptamer.

FIG. 2C depicts the control plasmid map which encodes the linear precursor RNA and PIE sequences used for RNA circularization but does not encode an aptamer. The plasmid elements are arranged in the following 5′ to 3′ order: a T7 promoter, a 5′ external homology arm, a 3′ Anabaena intron/exon fragment, a 5′ internal homology arm, a 5′ polyAC spacer, a CVB3 IRES, a protein coding region, a 3′ polyAC spacer, a 3′ internal homology arm, a 5′ Anabaena intron/exon fragment, and a 3′ external homology arm.

Each construct described in FIG. 2A-2C was driven by a T7 promoter and each plasmid contained a HindIII restriction site.

The subsequent examples test the generation and functionality of aptamer tagged circRNA constructs in streptavidin sepharose bead affinity purification.

### Example 2: Generation of Aptamer Tagged circRNA from Aptamer Tagged Linear Precursor RNA

The linear precursor RNA was synthesized by obtaining the cDNA template for IVT template via the linearization of the plasmids described in Example 1 using restriction enzyme, HindIII. Linearized template DNA was loaded into the IVT reaction for the experimental groups, 4xS1m aptamer tagged and tRNAxS1m aptamer tagged linear precursor RNA as well as the control group was carried out using the HiScribe T7 High Yield RNA Synthesis Kit (New England Biolabs) according to manufacturer's instructions.

After IVT reactions, samples were treated with DNase I (NEB) for 15 min. After DNase treatment, circRNA was generated from the linear precursor RNA by adding 2 mM GTP to IVT product and incubating at 55° C. for 15 min (i.e., circularization conditions). RNA samples were subsequently purified using LiCl precipitation and resuspended in 100 μl DEPC H2O.

After circularization conditions, three RNA species were expected to emerge from each respective sample: (1) aptamer-tagged circRNA, (2) residual aptamer-tagged linear precursor RNA that did not successfully undergo circularization, and (3) nicked aptamer-tagged circRNA. As previously reported, nicked aptamer-tagged circRNA is likely mediated by magnesium-catalyzed autohydrolysis which reduces the yield of the circRNA and is a deficiency that requires further optimization and improvement. Wesselhoeft et al., (2018), Nat Commun., 9 (1): 2629; Wesselhoeft et al., (2019), Mol Cell., 74 (3): 508-520; Li and Breaker, (1999), J. Am. Chem. Soc 121 (23): 5364-5372.

### Example 3: Streptavidin Sepharose Bead Affinity Purification and circRNA Quantification

Samples which had been subjected to the circulation conditions in Example 2 were tested in a Sepharose bead affinity purification strategy followed by quantification of the yield of RNA recovery.

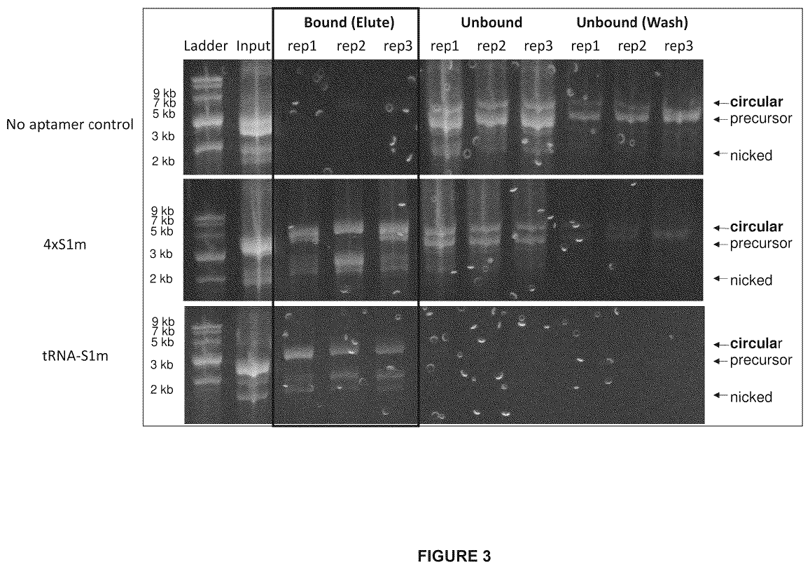

Methods for preparing the samples and binding conditions involved are disclosed in the following steps: (1) Preparation of the streptavidin Sepharose beads. To remove bead storage solution, 20 μL of streptavidin Sepharose beads (per sample) were spun at 0.8×g for 1 minute at 4° C. Subsequently, the beads were resuspended in 20 μL binding buffer and incubated on ice for 15 minutes. (2) Preparation of RNA aptamer tagged circRNA containing samples and incubation conditions. 2.5 μg of each sample was resuspended in 10 μL binding buffer. Refolding to allow aptamer to take on the expected secondary structure was performed by heating at 56° C. for 5 min, 37° C. for 10 min, and incubating at room temperature for 5 minutes. 2 μL of the sample was collected before binding to the sepharose beads and used as the control for input concentration. 10 μL of refolded aptamer (2.5 μg) were added to the Sepharose beads, incubated, and rotated at 4° C. for 2 hours. Beads were washed 2 times with 100 μL of binding buffer. (3) Elution of RNA aptamers from beads. Elution was performed with 250 μL phenol-based reagent in the following steps: 50 μL cold chloroform was added to the samples and vigorously shaken for 10 seconds. Subsequently, samples were spun at 12,000×g for 15 minutes at 4° C. Top aqueous phase (˜125 μL) containing RNA was directly transferred to Monarch cleanup columns and follow manufacturer's instructions, and finally eluted from Monarch column in 40 L DEPC H2O. (4) Quantification of yield of RNA recovery. RNA concentration following streptavidin affinity purification was quantified on a nanodrop. Elution, unbound, and wash fractions were run on a 2% EX Agarose Gel on an E-Gel Power Snap Electrophoresis system to visualize the RNA species present (aptamer-tagged circRNA, aptamer-tagged linear precursor RNA, and nicked RNA) in each of the fractions. Putative circRNA runs at a higher molecular weight than heavier linear precursor RNA, as indicated in FIG. 3.

As shown in FIG. 3, 4xS1m and tRNA-S1m aptamer tagged circRNA successfully underwent streptavidin Sepharose bead affinity purification relative to the no aptamer control sample (see lanes 3-5 containing eluted sample) and unbound fractions (compare lanes 3-5 with lanes 6-11). As predicted in Example 2, FIG. 3 also shows that circularization conditions resulted in three distinct RNA species (labeled on the agarose gel as “circular”, “precursor”, and “nicked”) indicating that the aptamer did not interfere with circularization of the linear precursor RNA.

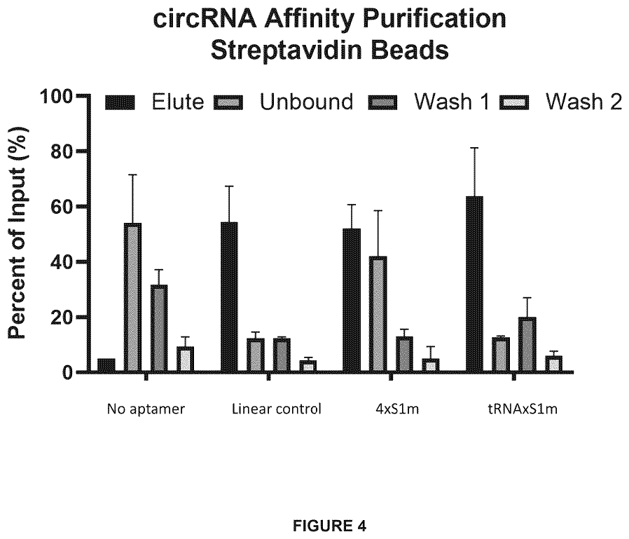

The amount of RNA recovery in each sample after streptavidin Sepharose bead affinity purification was also quantified. The results are shown in the bar graph of FIG. 4 which also displays an additional aptamer tagged linear precursor RNA control. Affinity purified 4xS1m aptamer tagged circRNA yielded approximately a 50% RNA recovery and the tRNAxS1m tagged circRNA yielded approximately a 60% RNA recovery yield relative to the input control sample. In contrast, the affinity purified control yielded approximately less than 5% RNA recovery yield. This result indicates that introducing aptamer tag to circRNA (e.g., a 4xS1m or a tRNAxS1m aptamer tag) can potentially be used to improve affinity purification efficiency of circRNA.

### Example 4: Negative Selection Scheme for Recovery of circRNA

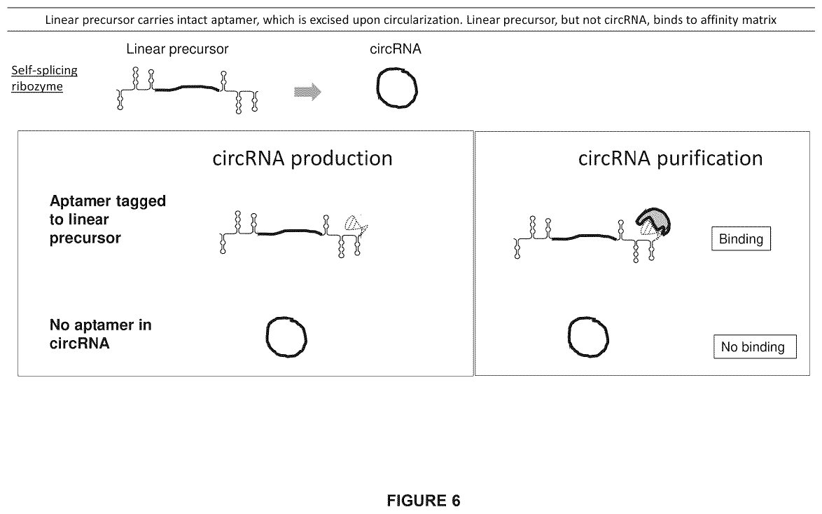

In Examples 1-3, aptamer-containing constructs were designed to be present in both the linear precursor RNA as well as the aptamer tagged circRNA (see FIG. 1). However, to optimally purify aptamer-tagged circRNA removal of the linear precursor RNA is necessary. Accordingly, linear precursor RNA were designed to create a negative selection strategy for affinity purification as diagrammed in FIG. 6.

Under the negative selection method, as shown in FIG. 6, the aptamer was localized in the linear precursor RNA at a position that would be removed upon circularization (i.e., the circRNA will not have the aptamer). In this configuration, the linear precursor RNA binds to the affinity matrix, but the circRNA does not.

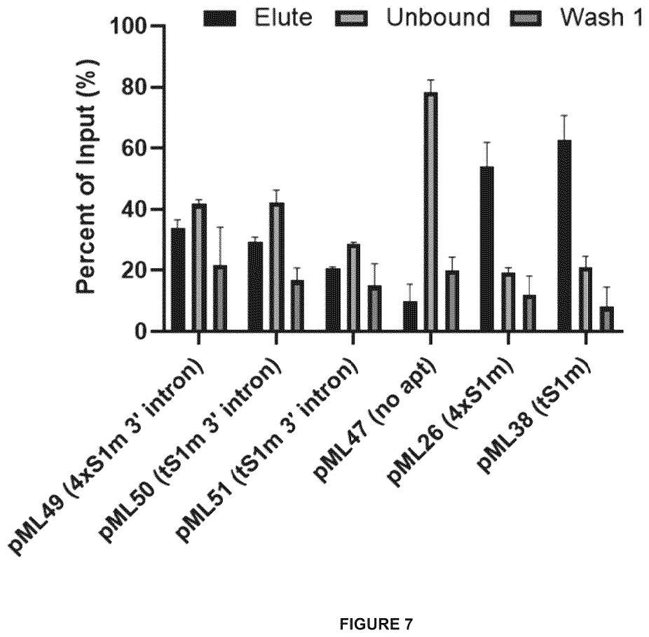

Several linear precursor RNAs were designed with the aptamer positioned at the 3′ intron region. After IVT and circularization, the circularization reaction mixture was incubated with streptavidin Sepharose beads as described above. The unbound, wash, and elution fractions were all collected. Purification of a 4xS1m aptamer tagged linear precursor RNA (pML49), a tRNA-S1m (tS1m) aptamer tagged linear precursor RNA (pML50 and pML51), a no aptamer control (pML47), a 4xS1m aptamer tagged circRNA (pML26), and a tRNA-S1m aptamer tagged circRNA (pML38) was performed. The amount of recovered RNA measured is expressed as a percent of the input (i.e., the input being the total RNA in the sample). As shown in FIG. 7, the negative selection constructs (pML49, pML50, pML51) showed binding that was intermediate between the no aptamer control (pML47) and the circRNA with aptamer designs (pML26 & pML38), suggesting that the portion of RNA in the unbound and wash fraction for the negative selection constructs was the desired circRNA.

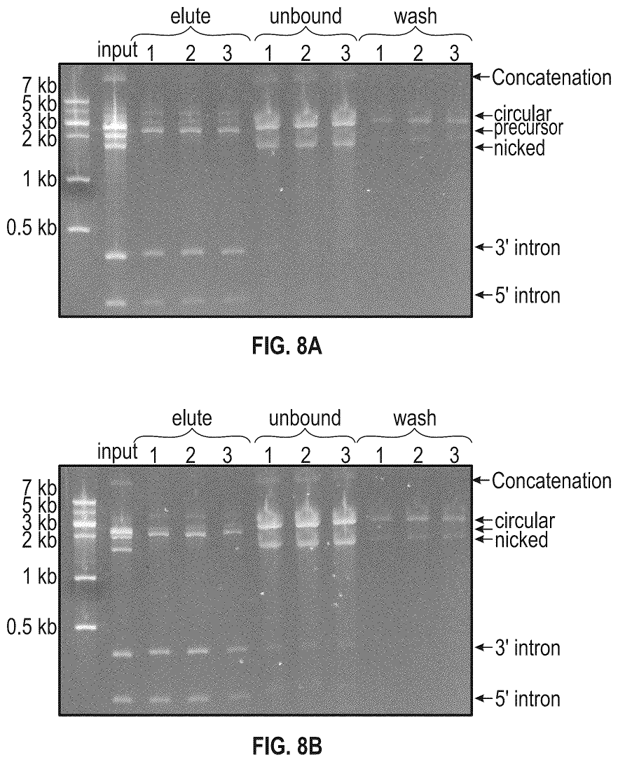

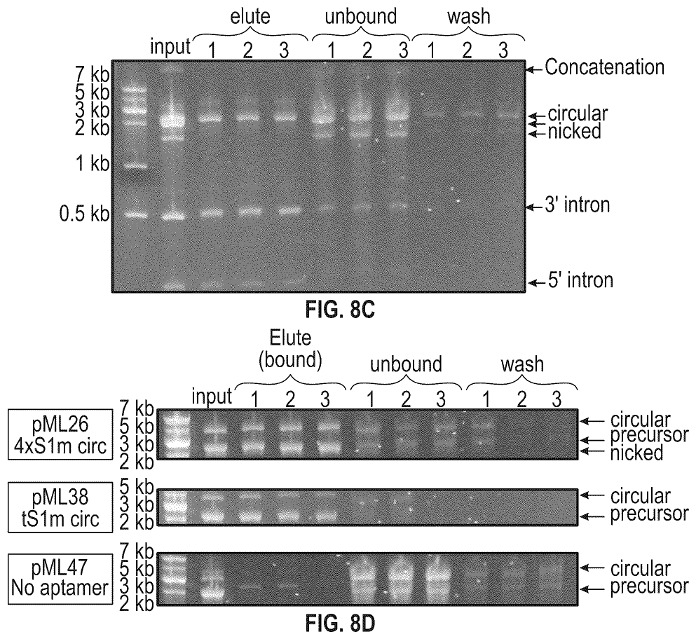

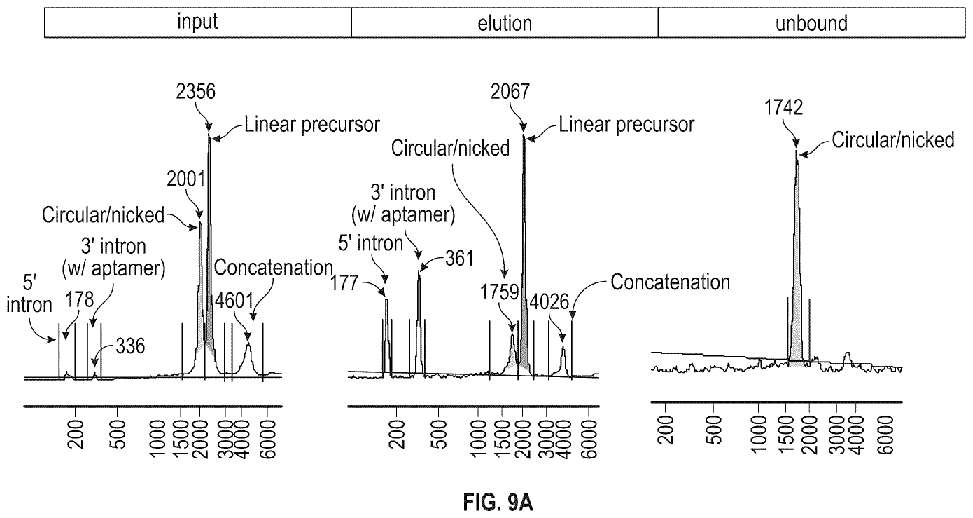

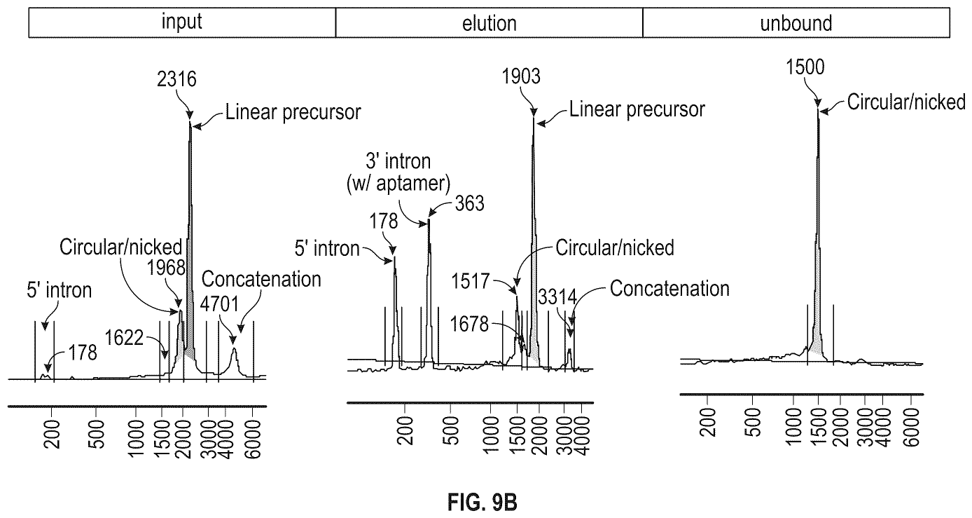

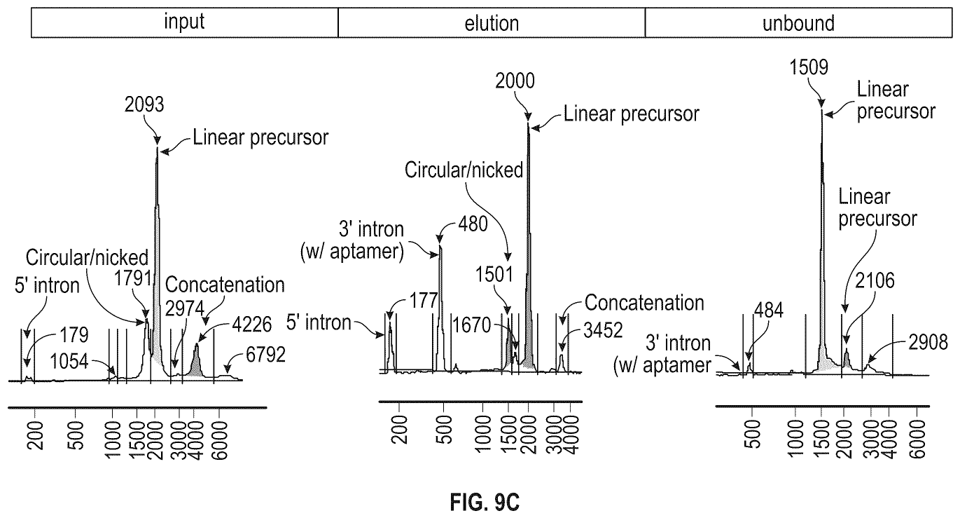

These results were analyzed further by taking images of agarose gels of the different samples. As shown in FIG. 8A-FIG. 8D, circRNA and nicked RNA species were predominantly found in the unbound and wash fraction, while linear precursor RNA was found in the eluted fraction for the negative selection constructs. A capillary electrophoresis assay was also performed to determine the various RNA species, as shown in FIG. 9A-FIG. 9C.

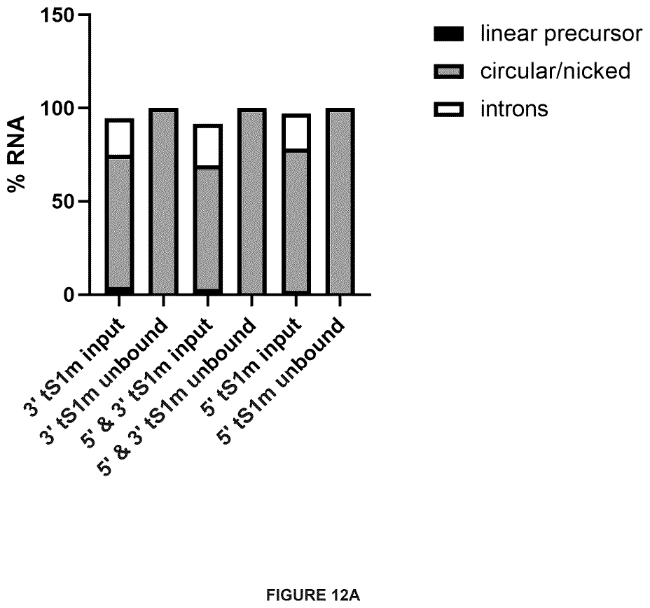

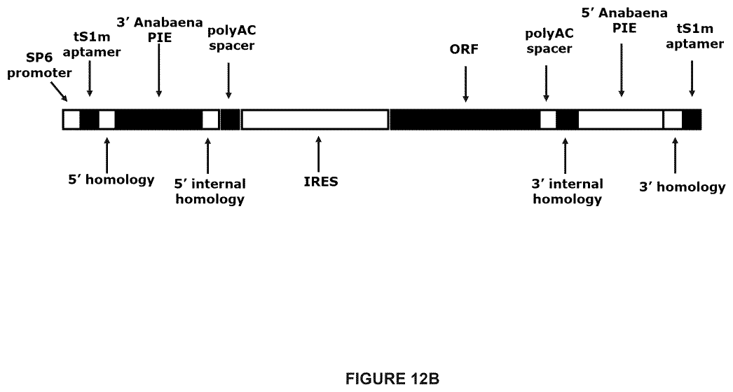

The placement of the aptamer in the linear precursor was tested. The tS1m aptamer was placed at the 3′ end of the linear precursor RNA (pML123), at the 5′ end of the linear precursor RNA (pML128), and at both the 5′ end and 3′ end of the linear precursor RNA (pML125). Each linear precursor RNA contained an ORF encoding for human erythropoietin (EPO), a gene of over 500 nucleotides. As shown in FIG. 12A-FIG. 12B, the placement or number of tS1m aptamers on the linear precursor did not negatively impact the purification of the circRNA. A summary of the purification is provided below in Table 1 for the pML125 construct. The introns in FIG. 12A results from the homology regions of the catalytic introns co-purifying when one of them contains the aptamer.

TABLE 1
Summary of purification

| | | | Total | | % | Total | Circular |
|---|---|---|---|---|---|---|---|
| Sample | Concentration | | RNA | | Circular | Circular | Recovery |
| Input | 217 | ng/ul | 8 | mg | 69% | 5.58 mg | N/A |
| Unbound | 504.7 | ng/ul | 2.019 | mg | 98% | 1.97 mg | 35.3% |
| Unbound + | 351.6 | ng/ul | 2.813 | mg | 98% | 2.76 mg | 49.5% |

washes

### Example 5: Positive Selection Scheme for Recovery of circRNA

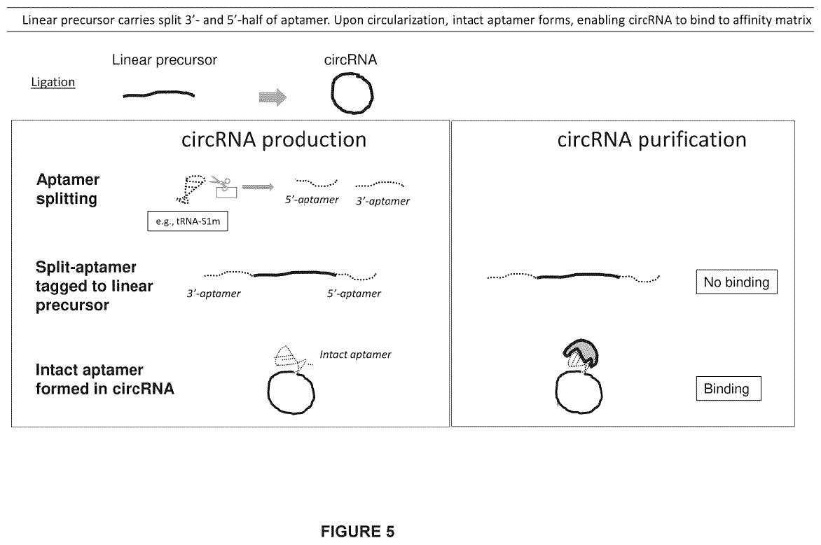

In Examples 1-3, aptamer-containing constructs were designed to be present in both the linear precursor RNA as well as the aptamer tagged circRNA (see FIG. 1). However, to optimally purify aptamer-tagged circRNA removal of the linear precursor RNA is necessary. Accordingly, linear precursor RNA were designed to create a positive selection strategy for affinity purification as diagrammed in FIG. 5.

Under the positive selection method, as shown in FIG. 5, a linear precursor RNA will be constructed to contain a split aptamer in which the 3′ and the 5′ half of the aptamer will be positioned at the 5′ and 3′ flanking ends of the linear precursor RNA, respectively. The linear precursor RNA will not undergo affinity purification because the intact aptamer is required for binding to the affinity matrix. Upon circularization of the linear precursor RNA, the intact aptamer will form allowing for binding to the affinity matrix.

cDNA templates will be generated and IVT will be used to produce the linear precursor RNA constructs. Constructs will vary the type of aptamer and its spatial configuration within the linear precursor RNA (see FIG. 5 for exemplary configurations). Table 2 shows the list of potential aptamer orientations for the tRNA-S1m and the 4xS1m aptamer in the linear precursor RNA. Upon completion of circularization conditions, constructs will be affinity purified using streptavidin sepharose beads and quantified as described in Example 3. Each construct will be evaluated based on RNA recovery relative to the input control sample.

### Example 6: Scale-Up of circRNA Purification

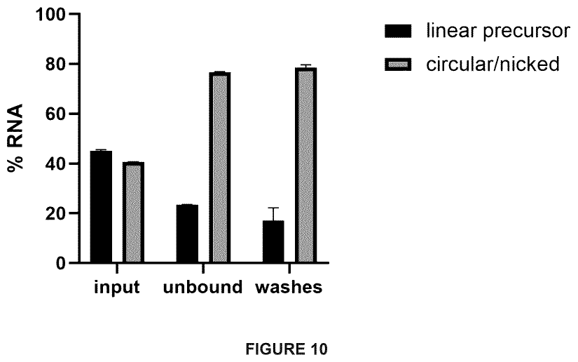

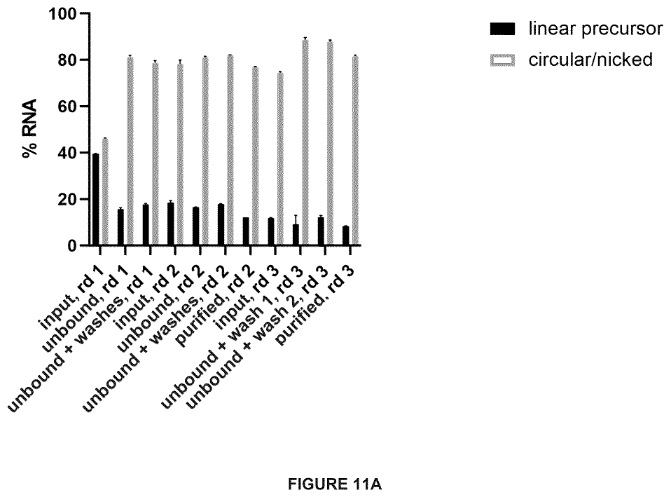

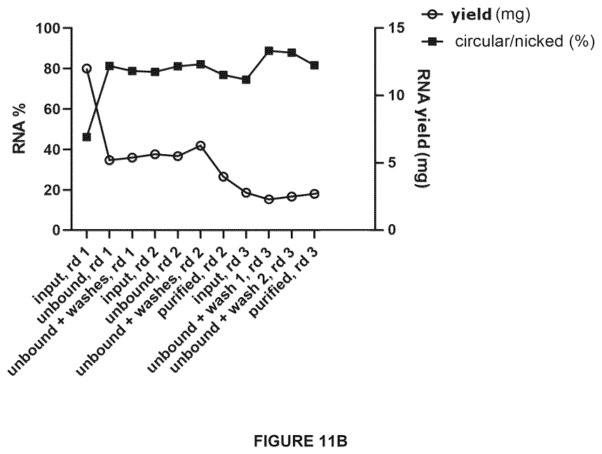

A scale up in the total input of linear precursor was performed to determine if the aptamer purification strategy would robustly purify the circRNA. As an initial matter, the template pML50 was modified to swap out the T7 RNA polymerase promoter for the SP6 promoter. An IVT reaction was performed to produce the linear precursor and the circularization reaction was performed with an initial 1 mg amount of RNA. As shown in FIG. 10, the 1 mg scale circularization followed by streptavidin purification yielded a highly pure circRNA in the unbound and wash fractions. Following the 1 mg scale purification, a larger 12 mg scale purification was attempted. In this assay, 3 rounds of the purification scheme were performed to increase purity. As shown in FIG. 11A, even at the higher starting amount of RNA, the circRNA was effectively purified, whether after 1, 2, or 3 rounds of purification. As shown in FIG. 11B, multiple rounds of purification yielded higher purities of circRNA.

### Example 7: Purification of Large circRNA

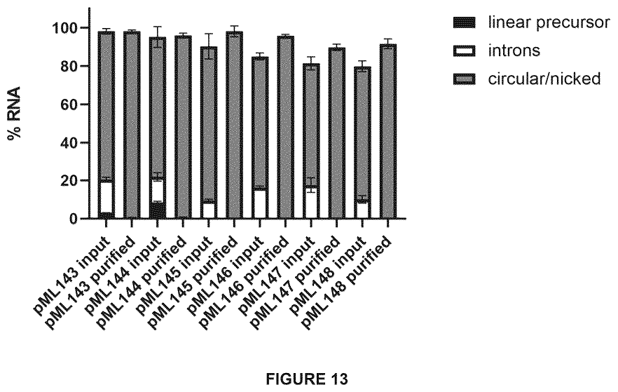

The circRNA purification strategies described above were attempted with circRNA encoding relatively small proteins (GFP and EPO). To test the efficacy of the aptamer purification strategy on larger circRNA, 6 different circRNA were generated with ORF sizes of 1032, 1035, 1725, 1728, 2172, and 2175 nucleotides. The full size of the 6 circRNAs were 1952, 2645, and 3092 nucleotides. As shown in FIG. 13, the 6 different constructs were purified through the negative selection purification scheme in which one or more aptamers are contained in the linear precursor, but lost during the circularization reaction. The data shows that the large circRNA was effectively purified.

### Example 8: Activity of circRNA Containing Aptamers

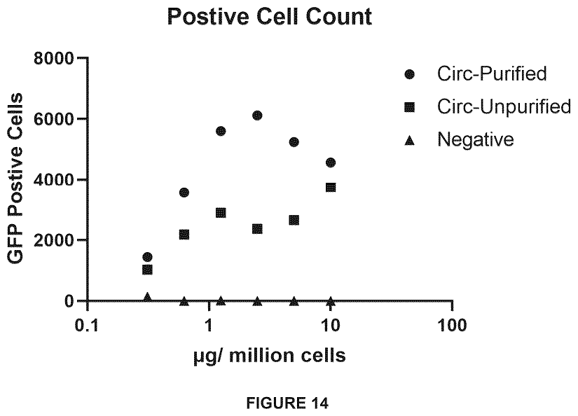

A circRNA was next tested to ensure expression of the encoded protein occurred. The pML50 circRNA encoding GFP was used, which was purified via the negative selection scheme, where the linear precursor RNA, but not he circRNA, contains the aptamer. The circRNA encoding GFP was transfected into Hela cells at different μg of RNA/million cells. As shown in FIG. 14, both purified and unpurified circRNA displayed GFP expression relative to a negative control., while the purified circRNA displayed greater expression relative to the unpurified circRNA.

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All patents and publications cited herein are incorporated by reference herein in their entirety.

TABLE 2
SEQUENCES
Linear precursor RNA-encoding nucleotide sequences

| SEQ ID NO/ | |
|---|---|
| Description | SEQUENCE |
| SEQ ID NO. | TAATACGACTCACTATAGGGGATCCGGGAGACCCTCGACCGTCGATTGTCCA |
| 1: | CTGGTCAACAATAGATGACTTACAACTAATCGGAAGGTGCAGAGACTCGACG |
| pML23_CVB | GGAGCTACCCTAACGTCAAGACGAGGGTAAAGAGAGAGTCCAATTCTCAAAG |
| 3-EGFP- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAACAAAACACATT |
| | AAAACAGCCTGTGGGTTGATCCCACCCACAGGCCCATTGGGCGCTAGCACTC |
| | TGGTATCACGGTACCTTTGTGCGCCTGTTTTATACCCCCTCCCCCAACTGTA |
| | ACTTAGAAGTAACACACACCGATCAACAGTCAGCGTGGCACACCAGCCACGT |
| | TTTGATCAAGCACTTCTGTTACCCCGGACTGAGTATCAATAGACTGCTCACG |
| | CGGTTGAAGGAGAAAGCGTTCGTTATCCGGCCAACTACTTCGAAAAACCTAG |
| | TAACACCGTGGAAGTTGCAGAGTGTTTCGCTCAGCACTACCCCAGTGTAGAT |
| | CAGGTCGATGAGTCACCGCATTCCCCACGGGCGACCGTGGCGGTGGCTGCGT |
| | TGGCGGCCTGCCCATGGGGAAACCCATGGGACGCTCTAATACAGACATGGTG |
| | CGAAGAGTCTATTGAGCTAGTTGGTAGTCCTCCGGCCCCTGAATGCGGCTAA |
| | TCCTAACTGCGGAGCACACACCCTCAAGCCAGAGGGCAGTGTGTCGTAACGG |
| | GCAACTCTGCAGCGGAACCGACTACTTTGGGTGTCCGTGTTTCATTTTATTC |
| | CTATACTGGCTGCTTATGGTGACAATTGAGAGATCGTTACCATATAGCTATT |
| | GGATTGGCCATCCGGTGACTAATAGAGCTATTATATATCCCTTTGTTGGGTT |
| | TATACCACTTAGCTTGAAAGAGGTTAAAACATTACAATTCATTGTTAAGTTG |
| | AATACAGCAAAATGGTGAGCAAGGGCGAGGAGCTGTTCACCGGGGTGGTGCC |
| | CATCCTGGTCGAGCTGGACGGCGACGTAAACGGCCACAAGTTCAGCGTGTCT |
| | GGCGAGGGCGAGGGCGATGCCACCTACGGCAAGCTGACCCTGAAGTTCATCT |
| | GCACCACCGGCAAGCTGCCCGTGCCCTGGCCCACCCTCGTGACCACCCTGAC |
| | CTACGGCGTGCAGTGCTTCAGCCGCTACCCCGACCACATGAAGCAGCACGAC |
| | TTCTTCAAGTCCGCCATGCCCGAAGGCTACGTCCAGGAGCGCACCATCTTCT |
| | TCAAGGACGACGGCAACTACAAGACCCGCGCCGAGGTGAAGTTCGAGGGCGA |
| | CACCCTGGTGAACCGCATCGAGCTGAAGGGCATCGACTTCAAGGAGGACGGC |
| | AACATCCTGGGGCACAAGCTGGAGTACAACTACAACAGCCACAACGTCTATA |
| | TCATGGCCGACAAGCAGAAGAACGGCATCAAGGCGAACTTCAAGATCCGCCA |
| | CAACATCGAGGACGGCAGCGTGCAGCTCGCCGACCACTACCAGCAGAACACC |
| | CCCATCGGCGACGGCCCCGTGCTGCTGCCCGACAACCACTACCTGAGCACCC |
| | AGTCCGCCCTGAGCAAAGACCCCAACGAGAAGCGCGATCACATGGTCCTGCT |
| | GGAGTTCGTGACCGCCGCCGGGATCACTCTCGGCATGGACGAGCTGTACAAG |
| | TAAAAAAAACAAAAAACAAAACGTAGAAAATGCGGCCGCCGACCAGAATCAT |
| | GCAAGTGCGTAAGATAGTCGCGGGTCGGCGGCCGCATCTGCTGGGAAGCTAC |
| | GATCCGTAGAAAATGCGGCCGCCGACCAGAATCATGCAAGTGCGTAAGATAG |
| | TCGCGGGTCGGCGGCCGCATCTGCTGGGTAGCTGTGAACCGTAGAAAATGCG |
| | GCCGCCGACCAGAATCATGCAAGTGCGTAAGATAGTCGCGGGTCGGCGGCCG |
| | CATCTGCTGGGAAGCTACGATCCGTAGAAAATGCGGCCGCCGACCAGAATCA |
| | TGCAAGTGCGTAAGATAGTCGCGGGTCGGCGGCCGCATCTGCTGGGGGCTAT |
| | TATGCGTTACCGGCGAGACGCTACGGACTTAAATAATTGAGCCTTAAAGAAG |
| | AAATTCTTTAAGTGGATGCTCTCAAACTCAGGGAAACCTAAATCTAGTTATA |
| | GACAAGGCAATCCTGAGCCAAGCCGAAGTAGTAATTAGTAAGACCAGTGGAC |
| | AATCGACG |
| SEQ ID NO. | TAATACGACTCACTATAGGGGATCCGGGAGACCCTCGACCGTCGATTGTCCA |
| 2: | CTGGTCAACAATAGATGACTTACAACTAATCGGAAGGTGCAGAGACTCGACG |
| pML24_CVB | GGAGCTACCCTAACGTCAAGACGAGGGTAAAGAGAGAGTCCAATTCTCAAAG |
| 3-EGFP- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAACAAAACACATT |
| | AAAACAGCCTGTGGGTTGATCCCACCCACAGGCCCATTGGGCGCTAGCACTC |
| | TGGTATCACGGTACCTTTGTGCGCCTGTTTTATACCCCCTCCCCCAACTGTA |
| | ACTTAGAAGTAACACACACCGATCAACAGTCAGCGTGGCACACCAGCCACGT |
| | TTTGATCAAGCACTTCTGTTACCCCGGACTGAGTATCAATAGACTGCTCACG |
| | CGGTTGAAGGAGAAAGCGTTCGTTATCCGGCCAACTACTTCGAAAAACCTAG |
| | TAACACCGTGGAAGTTGCAGAGTGTTTCGCTCAGCACTACCCCAGTGTAGAT |
| | CAGGTCGATGAGTCACCGCATTCCCCACGGGCGACCGTGGCGGTGGCTGCGT |
| | TGGCGGCCTGCCCATGGGGAAACCCATGGGACGCTCTAATACAGACATGGTG |
| | CGAAGAGTCTATTGAGCTAGTTGGTAGTCCTCCGGCCCCTGAATGCGGCTAA |
| | TCCTAACTGCGGAGCACACACCCTCAAGCCAGAGGGCAGTGTGTCGTAACGG |
| | GCAACTCTGCAGCGGAACCGACTACTTTGGGTGTCCGTGTTTCATTTTATTC |
| | CTATACTGGCTGCTTATGGTGACAATTGAGAGATCGTTACCATATAGCTATT |
| | GGATTGGCCATCCGGTGACTAATAGAGCTATTATATATCCCTTTGTTGGGTT |
| | TATACCACTTAGCTTGAAAGAGGTTAAAACATTACAATTCATTGTTAAGTTG |
| | AATACAGCAAAATGGTGAGCAAGGGCGAGGAGCTGTTCACCGGGGTGGTGCC |
| | CATCCTGGTCGAGCTGGACGGCGACGTAAACGGCCACAAGTTCAGCGTGTCT |
| | GGCGAGGGCGAGGGCGATGCCACCTACGGCAAGCTGACCCTGAAGTTCATCT |
| | GCACCACCGGCAAGCTGCCCGTGCCCTGGCCCACCCTCGTGACCACCCTGAC |
| | CTACGGCGTGCAGTGCTTCAGCCGCTACCCCGACCACATGAAGCAGCACGAC |
| | TTCTTCAAGTCCGCCATGCCCGAAGGCTACGTCCAGGAGCGCACCATCTTCT |
| | TCAAGGACGACGGCAACTACAAGACCCGCGCCGAGGTGAAGTTCGAGGGCGA |
| | CACCCTGGTGAACCGCATCGAGCTGAAGGGCATCGACTTCAAGGAGGACGGC |
| | AACATCCTGGGGCACAAGCTGGAGTACAACTACAACAGCCACAACGTCTATA |
| | TCATGGCCGACAAGCAGAAGAACGGCATCAAGGCGAACTTCAAGATCCGCCA |
| | CAACATCGAGGACGGCAGCGTGCAGCTCGCCGACCACTACCAGCAGAACACC |
| | CCCATCGGCGACGGCCCCGTGCTGCTGCCCGACAACCACTACCTGAGCACCC |
| | AGTCCGCCCTGAGCAAAGACCCCAACGAGAAGCGCGATCACATGGTCCTGCT |
| | GGAGTTCGTGACCGCCGCCGGGATCACTCTCGGCATGGACGAGCTGTACAAG |
| | TAAAAAAAACAAAAAACAAAACGTAGAAAATGCGGCCGCCGACCAGAATCAT |
| | GCAAGTGCGTAAGATAGTCGCGGGTCGGCGGCCGCATCTGCTGGGAAGCTAC |
| | GATCCGTAGAAAATGCGGCCGCCGACCAGAATCATGCAAGTGCGTAAGATAG |
| | TCGCGGGTCGGCGGCCGCATCTGCTGGGTAGCTGTGAACCGTAGAAAATGCG |
| | GCCGCCGACCAGAATCATGCAAGTGCGTAAGATAGTCGCGGGTCGGCGGCCG |
| | CATCTGCTGGGAAGCTACGATCCGTAGAAAATGCGGCCGCCGACCAGAATCA |
| | TGCAAGTGCGTAAGATAGTCGCGGGTCGGCGGCCGCATCTGCTGGGAGCTCG |
| | CTTTCTTGCTGTCCAATTTCTATTAAAGGTTCCTTTGTTCCCTAAGTCCAAC |
| | TACTAAACTGGGGGATATTATGAAGGGCCTTGAGCATCTGGATTCTGCCTAA |
| | TAAAAAACATTTATTTTCATTGCAGCTCGCTTTCTTGCTGTCCAATTTCTAT |
| | TAAAGGTTCCTTTGTTCCCTAAGTCCAACTACTAAACTGGGGGATATTATGA |
| | AGGGCCTTGAGCATCTGGATTCTGCCTAATAAAAAACATTTATTTTCATTGC |
| | GGCTATTATGCGTTACCGGCGAGACGCTACGGACTTAAATAATTGAGCCTTA |
| | AAGAAGAAATTCTTTAAGTGGATGCTCTCAAACTCAGGGAAACCTAAATCTA |
| | GTTATAGACAAGGCAATCCTGAGCCAAGCCGAAGTAGTAATTAGTAAGACCA |
| | GTGGACAATCGACG |
| SEQ ID NO. | TAATACGACTCACTATAGGGGATCCAGAGCGGCCGCTTTTTCAGCAAGATTA |
| 3: | AGCCCAGGGCAGAGCCATCTATTGOTTACATTTGCTTCTGACACAACTGTGT |
| pML25_UTR- | TCACTAGCAACCTCAAACAGACACCGGGAGACCCTCGACCGTCGATTGTCCA |
| CVB3-E | CTGGTCAACAATAGATGACTTACAACTAATCGGAAGGTGCAGAGACTCGACG |
| | GGAGCTACCCTAACGTCAAGACGAGGGTAAAGAGAGAGTCCAATTCTCAAAG |
| | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAACAAAACACATT |
| | AAAACAGCCTGTGGGTTGATCCCACCCACAGGCCCATTGGGCGCTAGCACTC |
| | TGGTATCACGGTACCTTTGTGCGCCTGTTTTATACCCCCTCCCCCAACTGTA |
| | ACTTAGAAGTAACACACACCGATCAACAGTCAGCGTGGCACACCAGCCACGT |
| | TTTGATCAAGCACTTCTGTTACCCCGGACTGAGTATCAATAGACTGCTCACG |
| | CGGTTGAAGGAGAAAGCGTTCGTTATCCGGCCAACTACTTCGAAAAACCTAG |
| | TAACACCGTGGAAGTTGCAGAGTGTTTCGCTCAGCACTACCCCAGTGTAGAT |
| | CAGGTCGATGAGTCACCGCATTCCCCACGGGCGACCGTGGCGGTGGCTGCGT |
| | TGGCGGCCTGCCCATGGGGAAACCCATGGGACGCTCTAATACAGACATGGTG |
| | CGAAGAGTCTATTGAGCTAGTTGGTAGTCCTCCGGCCCCTGAATGCGGCTAA |
| | TCCTAACTGCGGAGCACACACCCTCAAGCCAGAGGGCAGTGTGTCGTAACGG |
| | GCAACTCTGCAGCGGAACCGACTACTTTGGGTGTCCGTGTTTCATTTTATTC |
| | CTATACTGGCTGCTTATGGTGACAATTGAGAGATCGTTACCATATAGCTATT |
| | GGATTGGCCATCCGGTGACTAATAGAGCTATTATATATCCCTTTGTTGGGTT |
| | TATACCACTTAGCTTGAAAGAGGTTAAAACATTACAATTCATTGTTAAGTTG |
| | AATACAGCAAAATGGTGAGCAAGGGCGAGGAGCTGTTCACCGGGGTGGTGCC |
| | CATCCTGGTCGAGCTGGACGGCGACGTAAACGGCCACAAGTTCAGCGTGTCT |
| | GGCGAGGGCGAGGGCGATGCCACCTACGGCAAGCTGACCCTGAAGTTCATCT |
| | GCACCACCGGCAAGCTGCCCGTGCCCTGGCCCACCCTCGTGACCACCCTGAC |
| | CTACGGCGTGCAGTGCTTCAGCCGCTACCCCGACCACATGAAGCAGCACGAC |
| | TTCTTCAAGTCCGCCATGCCCGAAGGCTACGTCCAGGAGCGCACCATCTTCT |
| | TCAAGGACGACGGCAACTACAAGACCCGCGCCGAGGTGAAGTTCGAGGGCGA |
| | CACCCTGGTGAACCGCATCGAGCTGAAGGGCATCGACTTCAAGGAGGACGGC |
| | AACATCCTGGGGCACAAGCTGGAGTACAACTACAACAGCCACAACGTCTATA |
| | TCATGGCCGACAAGCAGAAGAACGGCATCAAGGCGAACTTCAAGATCCGCCA |
| | CAACATCGAGGACGGCAGCGTGCAGCTCGCCGACCACTACCAGCAGAACACC |
| | CCCATCGGCGACGGCCCCGTGCTGCTGCCCGACAACCACTACCTGAGCACCC |
| | AGTCCGCCCTGAGCAAAGACCCCAACGAGAAGCGCGATCACATGGTCCTGCT |
| | GGAGTTCGTGACCGCCGCCGGGATCACTCTCGGCATGGACGAGCTGTACAAG |
| | TAAAAAAAACAAAAAACAAAACGTAGAAAATGCGGCCGCCGACCAGAATCAT |
| | GCAAGTGCGTAAGATAGTCGCGGGTCGGCGGCCGCATCTGCTGGGAAGCTAC |
| | GATCCGTAGAAAATGCGGCCGCCGACCAGAATCATGCAAGTGCGTAAGATAG |
| | TCGCGGGTCGGCGGCCGCATCTGCTGGGTAGCTGTGAACCGTAGAAAATGCG |
| | GCCGCCGACCAGAATCATGCAAGTGCGTAAGATAGTCGCGGGTCGGCGGCCG |
| | CATCTGCTGGGAAGCTACGATCCGTAGAAAATGCGGCCGCCGACCAGAATCA |
| | TGCAAGTGCGTAAGATAGTCGCGGGTCGGCGGCCGCATCTGCTGGGAGCTCG |
| | CTTTCTTGCTGTCCAATTTCTATTAAAGGTTCCTTTGTTCCCTAAGTCCAAC |
| | TACTAAACTGGGGGATATTATGAAGGGCCTTGAGCATCTGGATTCTGCCTAA |
| | TAAAAAACATTTATTTTCATTGCAGCTCGCTTTCTTGCTGTCCAATTTCTAT |
| | TAAAGGTTCCTTTGTTCCCTAAGTCCAACTACTAAACTGGGGGATATTATGA |
| | AGGGCCTTGAGCATCTGGATTCTGCCTAATAAAAAACATTTATTTTCATTGC |
| | GGCTATTATGCGTTACCGGCGAGACGCTACGGACTTAAATAATTGAGCCTTA |
| | AAGAAGAAATTCTTTAAGTGGATGCTCTCAAACTCAGGGAAACCTAAATCTA |
| | GTTATAGACAAGGCAATCCTGAGCCAAGCCGAAGTAGTAATTAGTAAGACCA |
| | GTGGACAATCGACG |
| SEQ ID NO. | TAATACGACTCACTATAGGGGATCCGGGAGACCCTCGACCGTCGATTGTCCA |
| 4: | CTGGTCAACAATAGATGACTTACAACTAATCGGAAGGTGCAGAGACTCGACG |
| pML26_CVB | GGAGCTACCCTAACGTCAAGACGAGGGTAAAGAGAGAGTCCAATTCTCAAAG |
| 3-EGFP- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAACAAAACACATT |
| | AAAACAGCCTGTGGGTTGATCCCACCCACAGGCCCATTGGGCGCTAGCACTC |
| | TGGTATCACGGTACCTTTGTGCGCCTGTTTTATACCCCCTCCCCCAACTGTA |
| | ACTTAGAAGTAACACACACCGATCAACAGTCAGCGTGGCACACCAGCCACGT |
| | TTTGATCAAGCACTTCTGTTACCCCGGACTGAGTATCAATAGACTGCTCACG |
| | CGGTTGAAGGAGAAAGCGTTCGTTATCCGGCCAACTACTTCGAAAAACCTAG |
| | TAACACCGTGGAAGTTGCAGAGTGTTTCGCTCAGCACTACCCCAGTGTAGAT |
| | CAGGTCGATGAGTCACCGCATTCCCCACGGGCGACCGTGGCGGTGGCTGCGT |
| | TGGCGGCCTGCCCATGGGGAAACCCATGGGACGCTCTAATACAGACATGGTG |
| | CGAAGAGTCTATTGAGCTAGTTGGTAGTCCTCCGGCCCCTGAATGCGGCTAA |
| | TCCTAACTGCGGAGCACACACCCTCAAGCCAGAGGGCAGTGTGTCGTAACGG |
| | GCAACTCTGCAGCGGAACCGACTACTTTGGGTGTCCGTGTTTCATTTTATTC |
| | CTATACTGGCTGCTTATGGTGACAATTGAGAGATCGTTACCATATAGCTATT |
| | GGATTGGCCATCCGGTGACTAATAGAGCTATTATATATCCCTTTGTTGGGTT |
| | TATACCACTTAGCTTGAAAGAGGTTAAAACATTACAATTCATTGTTAAGTTG |
| | AATACAGCAAAATGGTGAGCAAGGGCGAGGAGCTGTTCACCGGGGTGGTGCC |
| | CATCCTGGTCGAGCTGGACGGCGACGTAAACGGCCACAAGTTCAGCGTGTCT |
| | GGCGAGGGCGAGGGCGATGCCACCTACGGCAAGCTGACCCTGAAGTTCATCT |
| | GCACCACCGGCAAGCTGCCCGTGCCCTGGCCCACCCTCGTGACCACCCTGAC |
| | CTACGGCGTGCAGTGCTTCAGCCGCTACCCCGACCACATGAAGCAGCACGAC |
| | TTCTTCAAGTCCGCCATGCCCGAAGGCTACGTCCAGGAGCGCACCATCTTCT |
| | TCAAGGACGACGGCAACTACAAGACCCGCGCCGAGGTGAAGTTCGAGGGCGA |
| | CACCCTGGTGAACCGCATCGAGCTGAAGGGCATCGACTTCAAGGAGGACGGC |
| | AACATCCTGGGGCACAAGCTGGAGTACAACTACAACAGCCACAACGTCTATA |
| | TCATGGCCGACAAGCAGAAGAACGGCATCAAGGCGAACTTCAAGATCCGCCA |
| | CAACATCGAGGACGGCAGCGTGCAGCTCGCCGACCACTACCAGCAGAACACC |
| | CCCATCGGCGACGGCCCCGTGCTGCTGCCCGACAACCACTACCTGAGCACCC |
| | AGTCCGCCCTGAGCAAAGACCCCAACGAGAAGCGCGATCACATGGTCCTGCT |
| | GGAGTTCGTGACCGCCGCCGGGATCACTCTCGGCATGGACGAGCTGTACAAG |
| | TAAGTAGAAAATGCGGCCGCCGACCAGAATCATGCAAGTGCGTAAGATAGTC |
| | GCGGGTCGGCGGCCGCATCTGCTGGGAAGCTACGATCCGTAGAAAATGCGGC |
| | CGCCGACCAGAATCATGCAAGTGCGTAAGATAGTCGCGGGTCGGCGGCCGCA |
| | TCTGCTGGGTAGCTGTGAACCGTAGAAAATGCGGCCGCCGACCAGAATCATG |
| | CAAGTGCGTAAGATAGTCGCGGGTCGGCGGCCGCATCTGCTGGGAAGCTACG |
| | ATCCGTAGAAAATGCGGCCGCCGACCAGAATCATGCAAGTGCGTAAGATAGT |
| | CGCGGGTCGGCGGCCGCATCTGCTGGGAAAAAACAAAAAACAAAACGGCTAT |
| | TATGCGTTACCGGCGAGACGCTACGGACTTAAATAATTGAGCCTTAAAGAAG |
| | AAATTCTTTAAGTGGATGCTCTCAAACTCAGGGAAACCTAAATCTAGTTATA |
| | GACAAGGCAATCCTGAGCCAAGCCGAAGTAGTAATTAGTAAGACCAGTGGAC |
| | AATCGACG |
| SEQ ID NO. | TAATACGACTCACTATAGGGGATCCGGGAGACCCTCGACCGTCGATTGTCCA |
| 5: | CTGGTCAACAATAGATGACTTACAACTAATCGGAAGGTGCAGAGACTCGACG |
| pML27_CVB | GGAGCTACCCTAACGTCAAGACGAGGGTAAAGAGAGAGTCCAATTCTCAAAG |
| 3-EGFP- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAACAAAACACATT |
| | AAAACAGCCTGTGGGTTGATCCCACCCACAGGCCCATTGGGCGCTAGCACTC |
| | TGGTATCACGGTACCTTTGTGCGCCTGTTTTATACCCCCTCCCCCAACTGTA |
| | ACTTAGAAGTAACACACACCGATCAACAGTCAGCGTGGCACACCAGCCACGT |
| | TTTGATCAAGCACTTCTGTTACCCCGGACTGAGTATCAATAGACTGCTCACG |
| | CGGTTGAAGGAGAAAGCGTTCGTTATCCGGCCAACTACTTCGAAAAACCTAG |
| | TAACACCGTGGAAGTTGCAGAGTGTTTCGCTCAGCACTACCCCAGTGTAGAT |
| | CAGGTCGATGAGTCACCGCATTCCCCACGGGCGACCGTGGCGGTGGCTGCGT |
| | TGGCGGCCTGCCCATGGGGAAACCCATGGGACGCTCTAATACAGACATGGTG |
| | CGAAGAGTCTATTGAGCTAGTTGGTAGTCCTCCGGCCCCTGAATGCGGCTAA |
| | TCCTAACTGCGGAGCACACACCCTCAAGCCAGAGGGCAGTGTGTCGTAACGG |
| | GCAACTCTGCAGCGGAACCGACTACTTTGGGTGTCCGTGTTTCATTTTATTC |
| | CTATACTGGCTGCTTATGGTGACAATTGAGAGATCGTTACCATATAGCTATT |
| | GGATTGGCCATCCGGTGACTAATAGAGCTATTATATATCCCTTTGTTGGGTT |
| | TATACCACTTAGCTTGAAAGAGGTTAAAACATTACAATTCATTGTTAAGTTG |
| | AATACAGCAAAATGGTGAGCAAGGGCGAGGAGCTGTTCACCGGGGTGGTGCC |
| | CATCCTGGTCGAGCTGGACGGCGACGTAAACGGCCACAAGTTCAGCGTGTCT |
| | GGCGAGGGCGAGGGCGATGCCACCTACGGCAAGCTGACCCTGAAGTTCATCT |
| | GCACCACCGGCAAGCTGCCCGTGCCCTGGCCCACCCTCGTGACCACCCTGAC |
| | CTACGGCGTGCAGTGCTTCAGCCGCTACCCCGACCACATGAAGCAGCACGAC |
| | TTCTTCAAGTCCGCCATGCCCGAAGGCTACGTCCAGGAGCGCACCATCTTCT |
| | TCAAGGACGACGGCAACTACAAGACCCGCGCCGAGGTGAAGTTCGAGGGCGA |
| | CACCCTGGTGAACCGCATCGAGCTGAAGGGCATCGACTTCAAGGAGGACGGC |
| | AACATCCTGGGGCACAAGCTGGAGTACAACTACAACAGCCACAACGTCTATA |
| | TCATGGCCGACAAGCAGAAGAACGGCATCAAGGCGAACTTCAAGATCCGCCA |
| | CAACATCGAGGACGGCAGCGTGCAGCTCGCCGACCACTACCAGCAGAACACC |
| | CCCATCGGCGACGGCCCCGTGCTGCTGCCCGACAACCACTACCTGAGCACCC |
| | AGTCCGCCCTGAGCAAAGACCCCAACGAGAAGCGCGATCACATGGTCCTGCT |
| | GGAGTTCGTGACCGCCGCCGGGATCACTCTCGGCATGGACGAGCTGTACAAG |
| | TAAGTAGAAAATGCGGCCGCCGACCAGAATCATGCAAGTGCGTAAGATAGTC |
| | GCGGGTCGGCGGCCGCATCTGCTGGGAAGCTACGATCCGTAGAAAATGCGGC |
| | CGCCGACCAGAATCATGCAAGTGCGTAAGATAGTCGCGGGTCGGCGGCCGCA |
| | TCTGCTGGGTAGCTGTGAACCGTAGAAAATGCGGCCGCCGACCAGAATCATG |
| | CAAGTGCGTAAGATAGTCGCGGGTCGGCGGCCGCATCTGCTGGGAAGCTACG |
| | ATCCGTAGAAAATGCGGCCGCCGACCAGAATCATGCAAGTGCGTAAGATAGT |
| | CGCGGGTCGGCGGCCGCATCTGCTGGGAGCTCGCTTTCTTGCTGTCCAATTT |
| | CTATTAAAGGTTCCTTTGTTCCCTAAGTCCAACTACTAAACTGGGGGATATT |
| | ATGAAGGGCCTTGAGCATCTGGATTCTGCCTAATAAAAAACATTTATTTTCA |
| | TTGCAGCTCGCTTTCTTGCTGTCCAATTTCTATTAAAGGTTCCTTTGTTCCC |
| | TAAGTCCAACTACTAAACTGGGGGATATTATGAAGGGCCTTGAGCATCTGGA |
| | TTCTGCCTAATAAAAAACATTTATTTTCATTGCAAAAAACAAAAAACAAAAC |
| | GGCTATTATGCGTTACCGGCGAGACGCTACGGACTTAAATAATTGAGCCTTA |
| | AAGAAGAAATTCTTTAAGTGGATGCTCTCAAACTCAGGGAAACCTAAATCTA |
| | GTTATAGACAAGGCAATCCTGAGCCAAGCCGAAGTAGTAATTAGTAAGACCA |
| | GTGGACAATCGACG |
| SEQ ID NO. | TAATACGACTCACTATAGGGGATCCGGGAGACCCTCGACCGTCGATTGTCCA |
| 6: | CTGGTCAACAATAGATGACTTACAACTAATCGGAAGGTGCAGAGACTCGACG |
| pML28_CVB | GGAGCTACCCTAACGTCAAGACGAGGGTAAAGAGAGAGTCCAATTCTCAAAG |
| 3-EGFP- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAACAAAACACATT |
| | AAAACAGCCTGTGGGTTGATCCCACCCACAGGCCCATTGGGCGCTAGCACTC |
| | TGGTATCACGGTACCTTTGTGCGCCTGTTTTATACCCCCTCCCCCAACTGTA |
| | ACTTAGAAGTAACACACACCGATCAACAGTCAGCGTGGCACACCAGCCACGT |
| | TTTGATCAAGCACTTCTGTTACCCCGGACTGAGTATCAATAGACTGCTCACG |
| | CGGTTGAAGGAGAAAGCGTTCGTTATCCGGCCAACTACTTCGAAAAACCTAG |
| | TAACACCGTGGAAGTTGCAGAGTGTTTCGCTCAGCACTACCCCAGTGTAGAT |
| | CAGGTCGATGAGTCACCGCATTCCCCACGGGCGACCGTGGCGGTGGCTGCGT |
| | TGGCGGCCTGCCCATGGGGAAACCCATGGGACGCTCTAATACAGACATGGTG |
| | CGAAGAGTCTATTGAGCTAGTTGGTAGTCCTCCGGCCCCTGAATGCGGCTAA |
| | TCCTAACTGCGGAGCACACACCCTCAAGCCAGAGGGCAGTGTGTCGTAACGG |
| | GCAACTCTGCAGCGGAACCGACTACTTTGGGTGTCCGTGTTTCATTTTATTC |
| | CTATACTGGCTGCTTATGGTGACAATTGAGAGATCGTTACCATATAGCTATT |
| | GGATTGGCCATCCGGTGACTAATAGAGCTATTATATATCCCTTTGTTGGGTT |
| | TATACCACTTAGCTTGAAAGAGGTTAAAACATTACAATTCATTGTTAAGTTG |
| | AATACAGCAAAATGGTGAGCAAGGGCGAGGAGCTGTTCACCGGGGTGGTGCC |
| | CATCCTGGTCGAGCTGGACGGCGACGTAAACGGCCACAAGTTCAGCGTGTCT |
| | GGCGAGGGCGAGGGCGATGCCACCTACGGCAAGCTGACCCTGAAGTTCATCT |
| | GCACCACCGGCAAGCTGCCCGTGCCCTGGCCCACCCTCGTGACCACCCTGAC |
| | CTACGGCGTGCAGTGCTTCAGCCGCTACCCCGACCACATGAAGCAGCACGAC |
| | TTCTTCAAGTCCGCCATGCCCGAAGGCTACGTCCAGGAGCGCACCATCTTCT |
| | TCAAGGACGACGGCAACTACAAGACCCGCGCCGAGGTGAAGTTCGAGGGCGA |
| | CACCCTGGTGAACCGCATCGAGCTGAAGGGCATCGACTTCAAGGAGGACGGC |
| | AACATCCTGGGGCACAAGCTGGAGTACAACTACAACAGCCACAACGTCTATA |
| | TCATGGCCGACAAGCAGAAGAACGGCATCAAGGCGAACTTCAAGATCCGCCA |
| | CAACATCGAGGACGGCAGCGTGCAGCTCGCCGACCACTACCAGCAGAACACC |
| | CCCATCGGCGACGGCCCCGTGCTGCTGCCCGACAACCACTACCTGAGCACCC |
| | AGTCCGCCCTGAGCAAAGACCCCAACGAGAAGCGCGATCACATGGTCCTGCT |
| | GGAGTTCGTGACCGCCGCCGGGATCACTCTCGGCATGGACGAGCTGTACAAG |
| | TAAAGCTCGCTTTCTTGCTGTCCAATTTCTATTAAAGGTTCCTTTGTTCCCT |
| | AAGTCCAACTACTAAACTGGGGGATATTATGAAGGGCCTTGAGCATCTGGAT |
| | TCTGCCTAATAAAAAACATTTATTTTCATTGCAGCTCGCTTTCTTGCTGTCC |
| | AATTTCTATTAAAGGTTCCTTTGTTCCCTAAGTCCAACTACTAAACTGGGGG |
| | ATATTATGAAGGGCCTTGAGCATCTGGATTCTGCCTAATAAAAAACATTTAT |
| | TTTCATTGCGTAGAAAATGCGGCCGCCGACCAGAATCATGCAAGTGCGTAAG |
| | ATAGTCGCGGGTCGGCGGCCGCATCTGCTGGGAAGCTACGATCCGTAGAAAA |
| | TGCGGCCGCCGACCAGAATCATGCAAGTGCGTAAGATAGTCGCGGGTCGGCG |
| | GCCGCATCTGCTGGGTAGCTGTGAACCGTAGAAAATGCGGCCGCCGACCAGA |
| | ATCATGCAAGTGCGTAAGATAGTCGCGGGTCGGCGGCCGCATCTGCTGGGAA |
| | GCTACGATCCGTAGAAAATGCGGCCGCCGACCAGAATCATGCAAGTGCGTAA |
| | GATAGTCGCGGGTCGGCGGCCGCATCTGCTGGGAAAAAACAAAAAACAAAAC |
| | GGCTATTATGCGTTACCGGCGAGACGCTACGGACTTAAATAATTGAGCCTTA |
| | AAGAAGAAATTCTTTAAGTGGATGCTCTCAAACTCAGGGAAACCTAAATCTA |
| | GTTATAGACAAGGCAATCCTGAGCCAAGCCGAAGTAGTAATTAGTAAGACCA |
| | GTGGACAATCGACG |
| SEQ ID NO. | TAATACGACTCACTATAGGGGATCCGGGAGACCCTCGACCGTCGATTGTCCA |
| 7: | CTGGTCAACAATAGATGACTTACAACTAATCGGAAGGTGCAGAGACTCGACG |
| pML29_CVB | GGAGCTACCCTAACGTCAAGACGAGGGTAAAGAGAGAGTCCAATTCTCAAAG |
| 3-GLuc- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAAAAAACACATTA |
| | AAACAGCCTGTGGGTTGATCCCACCCACAGGCCCATTGGGCGCTAGCACTCT |
| | GGTATCACGGTACCTTTGTGCGCCTGTTTTATACCCCCTCCCCCAACTGTAA |
| | CTTAGAAGTAACACACACCGATCAACAGTCAGCGTGGCACACCAGCCACGTT |
| | TTGATCAAGCACTTCTGTTACCCCGGACTGAGTATCAATAGACTGCTCACGC |
| | GGTTGAAGGAGAAAGCGTTCGTTATCCGGCCAACTACTTCGAAAAACCTAGT |
| | AACACCGTGGAAGTTGCAGAGTGTTTCGCTCAGCACTACCCCAGTGTAGATC |
| | AGGTCGATGAGTCACCGCATTCCCCACGGGCGACCGTGGCGGTGGCTGCGTT |
| | GGCGGCCTGCCCATGGGGAAACCCATGGGACGCTCTAATACAGACATGGTGC |
| | GAAGAGTCTATTGAGCTAGTTGGTAGTCCTCCGGCCCCTGAATGCGGCTAAT |
| | CCTAACTGCGGAGCACACACCCTCAAGCCAGAGGGCAGTGTGTCGTAACGGG |
| | CAACTCTGCAGCGGAACCGACTACTTTGGGTGTCCGTGTTTCATTTTATTCC |
| | TATACTGGCTGCTTATGGTGACAATTGAGAGATCGTTACCATATAGCTATTG |
| | GATTGGCCATCCGGTGACTAATAGAGCTATTATATATCCCTTTGTTGGGTTT |
| | ATACCACTTAGCTTGAAAGAGGTTAAAACATTACAATTCATTGTTAAGTTGA |
| | ATACAGCAAAATGGGAGTCAAAGTTCTGTTTGCCCTGATCTGCATCGCTGTG |
| | GCCGAGGCCAAGCCCACCGAGAACAACGAAGACTTCAACATCGTGGCCGTGG |
| | CCAGCAACTTCGCGACCACGGATCTCGATGCTGACCGCGGGAAGTTGCCCGG |
| | CAAGAAGCTGCCGCTGGAGGTGCTCAAAGAGATGGAAGCCAATGCCCGGAAA |
| | GCTGGCTGCACCAGGGGCTGTCTGATCTGCCTGTCCCACATCAAGTGCACGC |
| | CCAAGATGAAGAAGTTCATCCCAGGACGCTGCCACACCTACGAAGGCGACAA |
| | AGAGTCCGCACAGGGGGGCATAGGCGAGGCGATCGTCGACATTCCTGAGATT |
| | CCTGGGTTCAAGGACTTGGAGCCCATGGAGCAGTTCATCGCACAGGTCGATC |
| | TGTGTGTGGACTGCACAACTGGCTGCCTCAAAGGGCTTGCCAACGTGCAGTG |
| | TTCTGACCTGCTCAAGAAGTGGCTGCCGCAACGCTGTGCGACCTTTGCCAGC |
| | AAGATCCAGGGCCAGGTGGACAAGATCAAGGGGGCCGGTGGTGACTAAAAAA |
| | AACAAAAAACAAAACGTAGAAAATGCGGCCGCCGACCAGAATCATGCAAGTG |
| | CGTAAGATAGTCGCGGGTCGGCGGCCGCATCTGCTGGGAAGCTACGATCCGT |
| | AGAAAATGCGGCCGCCGACCAGAATCATGCAAGTGCGTAAGATAGTCGCGGG |
| | TCGGCGGCCGCATCTGCTGGGTAGCTGTGAACCGTAGAAAATGCGGCCGCCG |
| | ACCAGAATCATGCAAGTGCGTAAGATAGTCGCGGGTCGGCGGCCGCATCTGC |
| | TGGGAAGCTACGATCCGTAGAAAATGCGGCCGCCGACCAGAATCATGCAAGT |
| | GCGTAAGATAGTCGCGGGTCGGCGGCCGCATCTGCTGGGGGCTATTATGCGT |
| | TACCGGCGAGACGCTACGGACTTAAATAATTGAGCCTTAAAGAAGAAATTCT |
| | TTAAGTGGATGCTCTCAAACTCAGGGAAACCTAAATCTAGTTATAGACAAGG |
| | CAATCCTGAGCCAAGCCGAAGTAGTAATTAGTAAGACCAGTGGACAATCGAC |
| | G |
| SEQ ID NO. | TAATACGACTCACTATAGGGGATCCGGGAGACCCTCGACCGTCGATTGTCCA |
| 8: | CTGGTCAACAATAGATGACTTACAACTAATCGGAAGGTGCAGAGACTCGACG |
| pML30_CVB | GGAGCTACCCTAACGTCAAGACGAGGGTAAAGAGAGAGTCCAATTCTCAAAG |
| 3-GLuc- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAACAAAACACATT |
| | AAAACAGCCTGTGGGTTGATCCCACCCACAGGCCCATTGGGCGCTAGCACTC |
| | TGGTATCACGGTACCTTTGTGCGCCTGTTTTATACCCCCTCCCCCAACTGTA |
| | ACTTAGAAGTAACACACACCGATCAACAGTCAGCGTGGCACACCAGCCACGT |
| | TTTGATCAAGCACTTCTGTTACCCCGGACTGAGTATCAATAGACTGCTCACG |
| | CGGTTGAAGGAGAAAGCGTTCGTTATCCGGCCAACTACTTCGAAAAACCTAG |
| | TAACACCGTGGAAGTTGCAGAGTGTTTCGCTCAGCACTACCCCAGTGTAGAT |
| | CAGGTCGATGAGTCACCGCATTCCCCACGGGCGACCGTGGCGGTGGCTGCGT |
| | TGGCGGCCTGCCCATGGGGAAACCCATGGGACGCTCTAATACAGACATGGTG |
| | CGAAGAGTCTATTGAGCTAGTTGGTAGTCCTCCGGCCCCTGAATGCGGCTAA |
| | TCCTAACTGCGGAGCACACACCCTCAAGCCAGAGGGCAGTGTGTCGTAACGG |
| | GCAACTCTGCAGCGGAACCGACTACTTTGGGTGTCCGTGTTTCATTTTATTC |
| | CTATACTGGCTGCTTATGGTGACAATTGAGAGATCGTTACCATATAGCTATT |
| | GGATTGGCCATCCGGTGACTAATAGAGCTATTATATATCCCTTTGTTGGGTT |
| | TATACCACTTAGCTTGAAAGAGGTTAAAACATTACAATTCATTGTTAAGTTG |
| | AATACAGCAAAATGGGAGTCAAAGTTCTGTTTGCCCTGATCTGCATCGCTGT |
| | GGCCGAGGCCAAGCCCACCGAGAACAACGAAGACTTCAACATCGTGGCCGTG |
| | GCCAGCAACTTCGCGACCACGGATCTCGATGCTGACCGCGGGAAGTTGCCCG |
| | GCAAGAAGCTGCCGCTGGAGGTGCTCAAAGAGATGGAAGCCAATGCCCGGAA |
| | AGCTGGCTGCACCAGGGGCTGTCTGATCTGCCTGTCCCACATCAAGTGCACG |
| | CCCAAGATGAAGAAGTTCATCCCAGGACGCTGCCACACCTACGAAGGCGACA |
| | AAGAGTCCGCACAGGGGGGCATAGGCGAGGCGATCGTCGACATTCCTGAGAT |
| | TCCTGGGTTCAAGGACTTGGAGCCCATGGAGCAGTTCATCGCACAGGTCGAT |
| | CTGTGTGTGGACTGCACAACTGGCTGCCTCAAAGGGCTTGCCAACGTGCAGT |
| | GTTCTGACCTGCTCAAGAAGTGGCTGCCGCAACGCTGTGCGACCTTTGCCAG |
| | CAAGATCCAGGGCCAGGTGGACAAGATCAAGGGGGCCGGTGGTGACTAAAAA |
| | AAACAAAAAACAAAACGTAGAAAATGCGGCCGCCGACCAGAATCATGCAAGT |
| | GCGTAAGATAGTCGCGGGTCGGCGGCCGCATCTGCTGGGAAGCTACGATCCG |
| | TAGAAAATGCGGCCGCCGACCAGAATCATGCAAGTGCGTAAGATAGTCGCGG |
| | GTCGGCGGCCGCATCTGCTGGGTAGCTGTGAACCGTAGAAAATGCGGCCGCC |
| | GACCAGAATCATGCAAGTGCGTAAGATAGTCGCGGGTCGGCGGCCGCATCTG |
| | CTGGGAAGCTACGATCCGTAGAAAATGCGGCCGCCGACCAGAATCATGCAAG |
| | TGCGTAAGATAGTCGCGGGTCGGCGGCCGCATCTGCTGGGAGCTCGCTTTCT |
| | TGCTGTCCAATTTCTATTAAAGGTTCCTTTGTTCCCTAAGTCCAACTACTAA |
| | ACTGGGGGATATTATGAAGGGCCTTGAGCATCTGGATTCTGCCTAATAAAAA |
| | ACATTTATTTTCATTGCAGCTCGCTTTCTTGCTGTCCAATTTCTATTAAAGG |
| | TTCCTTTGTTCCCTAAGTCCAACTACTAAACTGGGGGATATTATGAAGGGCC |
| | TTGAGCATCTGGATTCTGCCTAATAAAAAACATTTATTTTCATTGCGGCTAT |
| | TATGCGTTACCGGCGAGACGCTACGGACTTAAATAATTGAGCCTTAAAGAAG |
| | AAATTCTTTAAGTGGATGCTCTCAAACTCAGGGAAACCTAAATCTAGTTATA |
| | GACAAGGCAATCCTGAGCCAAGCCGAAGTAGTAATTAGTAAGACCAGTGGAC |
| | AATCGACG |
| SEQ ID NO. | TAATACGACTCACTATAGGGGATCCGGGAGACCCTCGACCGTCGATTGTCCA |
| 9: | CTGGTCAACAATAGATGACTTACAACTAATCGGAAGGTGCAGAGACTCGACG |
| pML31_CVB | GGAGCTACCCTAACGTCAAGACGAGGGTAAAGAGAGAGTCCAATTCTCAAAG |
| 3-GLuc- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAACAAAACACATT |
| | AAAACAGCCTGTGGGTTGATCCCACCCACAGGCCCATTGGGCGCTAGCACTC |
| | TGGTATCACGGTACCTTTGTGCGCCTGTTTTATACCCCCTCCCCCAACTGTA |
| | ACTTAGAAGTAACACACACCGATCAACAGTCAGCGTGGCACACCAGCCACGT |
| | TTTGATCAAGCACTTCTGTTACCCCGGACTGAGTATCAATAGACTGCTCACG |
| | CGGTTGAAGGAGAAAGCGTTCGTTATCCGGCCAACTACTTCGAAAAACCTAG |
| | TAACACCGTGGAAGTTGCAGAGTGTTTCGCTCAGCACTACCCCAGTGTAGAT |
| | CAGGTCGATGAGTCACCGCATTCCCCACGGGCGACCGTGGCGGTGGCTGCGT |
| | TGGCGGCCTGCCCATGGGGAAACCCATGGGACGCTCTAATACAGACATGGTG |
| | CGAAGAGTCTATTGAGCTAGTTGGTAGTCCTCCGGCCCCTGAATGCGGCTAA |
| | TCCTAACTGCGGAGCACACACCCTCAAGCCAGAGGGCAGTGTGTCGTAACGG |
| | GCAACTCTGCAGCGGAACCGACTACTTTGGGTGTCCGTGTTTCATTTTATTC |
| | CTATACTGGCTGCTTATGGTGACAATTGAGAGATCGTTACCATATAGCTATT |
| | GGATTGGCCATCCGGTGACTAATAGAGCTATTATATATCCCTTTGTTGGGTT |
| | TATACCACTTAGCTTGAAAGAGGTTAAAACATTACAATTCATTGTTAAGTTG |
| | AATACAGCAAAATGGGAGTCAAAGTTCTGTTTGCCCTGATCTGCATCGCTGT |
| | GGCCGAGGCCAAGCCCACCGAGAACAACGAAGACTTCAACATCGTGGCCGTG |
| | GCCAGCAACTTCGCGACCACGGATCTCGATGCTGACCGCGGGAAGTTGCCCG |
| | GCAAGAAGCTGCCGCTGGAGGTGCTCAAAGAGATGGAAGCCAATGCCCGGAA |
| | AGCTGGCTGCACCAGGGGCTGTCTGATCTGCCTGTCCCACATCAAGTGCACG |
| | CCCAAGATGAAGAAGTTCATCCCAGGACGCTGCCACACCTACGAAGGCGACA |
| | AAGAGTCCGCACAGGGGGGCATAGGCGAGGCGATCGTCGACATTCCTGAGAT |
| | TCCTGGGTTCAAGGACTTGGAGCCCATGGAGCAGTTCATCGCACAGGTCGAT |
| | CTGTGTGTGGACTGCACAACTGGCTGCCTCAAAGGGCTTGCCAACGTGCAGT |
| | GTTCTGACCTGCTCAAGAAGTGGCTGCCGCAACGCTGTGCGACCTTTGCCAG |
| | CAAGATCCAGGGCCAGGTGGACAAGATCAAGGGGGCCGGTGGTGACTAAGTA |
| | GAAAATGCGGCCGCCGACCAGAATCATGCAAGTGCGTAAGATAGTCGCGGGT |
| | CGGCGGCCGCATCTGCTGGGAAGCTACGATCCGTAGAAAATGCGGCCGCCGA |
| | CCAGAATCATGCAAGTGCGTAAGATAGTCGCGGGTCGGCGGCCGCATCTGCT |
| | GGGTAGCTGTGAACCGTAGAAAATGCGGCCGCCGACCAGAATCATGCAAGTG |
| | CGTAAGATAGTCGCGGGTCGGCGGCCGCATCTGCTGGGAAGCTACGATCCGT |
| | AGAAAATGCGGCCGCCGACCAGAATCATGCAAGTGCGTAAGATAGTCGCGGG |
| | TCGGCGGCCGCATCTGCTGGGAAAAAACAAAAAACAAAACGGCTATTATGCG |
| | TTACCGGCGAGACGCTACGGACTTAAATAATTGAGCCTTAAAGAAGAAATTC |
| | TTTAAGTGGATGCTCTCAAACTCAGGGAAACCTAAATCTAGTTATAGACAAG |
| | GCAATCCTGAGCCAAGCCGAAGTAGTAATTAGTAAGACCAGTGGACAATCGA |
| | CG |
| SEQ ID NO. | TAATACGACTCACTATAGGGGATCCGGGAGACCCTCGACCGTCGATTGTCCA |
| 10: | CTGGTCAACAATAGATGACTTACAACTAATCGGAAGGTGCAGAGACTCGACG |
| pML32_CVB | GGAGCTACCCTAACGTCAAGACGAGGGTAAAGAGAGAGTCCAATTCTCAAAG |
| 3-GLuc- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAACAAAACACATT |
| | AAAACAGCCTGTGGGTTGATCCCACCCACAGGCCCATTGGGCGCTAGCACTC |
| | TGGTATCACGGTACCTTTGTGCGCCTGTTTTATACCCCCTCCCCCAACTGTA |
| | ACTTAGAAGTAACACACACCGATCAACAGTCAGCGTGGCACACCAGCCACGT |
| | TTTGATCAAGCACTTCTGTTACCCCGGACTGAGTATCAATAGACTGCTCACG |
| | CGGTTGAAGGAGAAAGCGTTCGTTATCCGGCCAACTACTTCGAAAAACCTAG |
| | TAACACCGTGGAAGTTGCAGAGTGTTTCGCTCAGCACTACCCCAGTGTAGAT |
| | CAGGTCGATGAGTCACCGCATTCCCCACGGGCGACCGTGGCGGTGGCTGCGT |
| | TGGCGGCCTGCCCATGGGGAAACCCATGGGACGCTCTAATACAGACATGGTG |
| | CGAAGAGTCTATTGAGCTAGTTGGTAGTCCTCCGGCCCCTGAATGCGGCTAA |
| | TCCTAACTGCGGAGCACACACCCTCAAGCCAGAGGGCAGTGTGTCGTAACGG |
| | GCAACTCTGCAGCGGAACCGACTACTTTGGGTGTCCGTGTTTCATTTTATTC |
| | CTATACTGGCTGCTTATGGTGACAATTGAGAGATCGTTACCATATAGCTATT |
| | GGATTGGCCATCCGGTGACTAATAGAGCTATTATATATCCCTTTGTTGGGTT |
| | TATACCACTTAGCTTGAAAGAGGTTAAAACATTACAATTCATTGTTAAGTTG |
| | AATACAGCAAAATGGGAGTCAAAGTTCTGTTTGCCCTGATCTGCATCGCTGT |
| | GGCCGAGGCCAAGCCCACCGAGAACAACGAAGACTTCAACATCGTGGCCGTG |
| | GCCAGCAACTTCGCGACCACGGATCTCGATGCTGACCGCGGGAAGTTGCCCG |
| | GCAAGAAGCTGCCGCTGGAGGTGCTCAAAGAGATGGAAGCCAATGCCCGGAA |
| | AGCTGGCTGCACCAGGGGCTGTCTGATCTGCCTGTCCCACATCAAGTGCACG |
| | CCCAAGATGAAGAAGTTCATCCCAGGACGCTGCCACACCTACGAAGGCGACA |
| | AAGAGTCCGCACAGGGCGGCATAGGCGAGGCGATCGTCGACATTCCTGAGAT |
| | TCCTGGGTTCAAGGACTTGGAGCCCATGGAGCAGTTCATCGCACAGGTCGAT |
| | CTGTGTGTGGACTGCACAACTGGCTGCCTCAAAGGGCTTGCCAACGTGCAGT |
| | GTTCTGACCTGCTCAAGAAGTGGCTGCCGCAACGCTGTGCGACCTTTGCCAG |
| | CAAGATCCAGGGCCAGGTGGACAAGATCAAGGGGGCCGGTGGTGACTAAAGC |
| | TCGCTTTCTTGCTGTCCAATTTCTATTAAAGGTTCCTTTGTTCCCTAAGTCC |
| | AACTACTAAACTGGGGGATATTATGAAGGGCCTTGAGCATCTGGATTCTGCC |
| | TAATAAAAAACATTTATTTTCATTGCAGCTCGCTTTCTTGCTGTCCAATTTC |
| | TATTAAAGGTTCCTTTGTTCCCTAAGTCCAACTACTAAACTGGGGGATATTA |
| | TGAAGGGCCTTGAGCATCTGGATTCTGCCTAATAAAAAACATTTATTTTCAT |
| | TGCGTAGAAAATGCGGCCGCCGACCAGAATCATGCAAGTGCGTAAGATAGTC |
| | GCGGGTCGGCGGCCGCATCTGCTGGGAAGCTACGATCCGTAGAAAATGCGGC |
| | CGCCGACCAGAATCATGCAAGTGCGTAAGATAGTCGCGGGTCGGCGGCCGCA |
| | TCTGCTGGGTAGCTGTGAACCGTAGAAAATGCGGCCGCCGACCAGAATCATG |
| | CAAGTGCGTAAGATAGTCGCGGGTCGGCGGCCGCATCTGCTGGGAAGCTACG |
| | ATCCGTAGAAAATGCGGCCGCCGACCAGAATCATGCAAGTGCGTAAGATAGT |
| | CGCGGGTCGGCGGCCGCATCTGCTGGGAAAAAACAAAAAACAAAACGGCTAT |
| | TATGCGTTACCGGCGAGACGCTACGGACTTAAATAATTGAGCCTTAAAGAAG |
| | AAATTCTTTAAGTGGATGCTCTCAAACTCAGGGAAACCTAAATCTAGTTATA |
| | GACAAGGCAATCCTGAGCCAAGCCGAAGTAGTAATTAGTAAGACCAGTGGAC |
| | AATCGACG |
| SEQ ID NO. | TAATACGACTCACTATAGGGGATCCGGGAGACCCTCGACCGTCGATTGTCCA |
| 11: | CTGGTCAACAATAGATGACTTACAACTAATCGGAAGGTGCAGAGACTCGACG |
| pML33_CVB | GGAGCTACCCTAACGTCAAGACGAGGGTAAAGAGAGAGTCCAATTCTCAAAG |
| 3-GLuc- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAACAAAACACATT |
| | AAAACAGCCTGTGGGTTGATCCCACCCACAGGCCCATTGGGCGCTAGCACTC |
| | TGGTATCACGGTACCTTTGTGCGCCTGTTTTATACCCCCTCCCCCAACTGTA |
| | ACTTAGAAGTAACACACACCGATCAACAGTCAGCGTGGCACACCAGCCACGT |
| | TTTGATCAAGCACTTCTGTTACCCCGGACTGAGTATCAATAGACTGCTCACG |
| | CGGTTGAAGGAGAAAGCGTTCGTTATCCGGCCAACTACTTCGAAAAACCTAG |
| | TAACACCGTGGAAGTTGCAGAGTGTTTCGCTCAGCACTACCCCAGTGTAGAT |
| | CAGGTCGATGAGTCACCGCATTCCCCACGGGCGACCGTGGCGGTGGCTGCGT |
| | TGGCGGCCTGCCCATGGGGAAACCCATGGGACGCTCTAATACAGACATGGTG |
| | CGAAGAGTCTATTGAGCTAGTTGGTAGTCCTCCGGCCCCTGAATGCGGCTAA |
| | TCCTAACTGCGGAGCACACACCCTCAAGCCAGAGGGCAGTGTGTCGTAACGG |
| | GCAACTCTGCAGCGGAACCGACTACTTTGGGTGTCCGTGTTTCATTTTATTC |
| | CTATACTGGCTGCTTATGGTGACAATTGAGAGATCGTTACCATATAGCTATT |
| | GGATTGGCCATCCGGTGACTAATAGAGCTATTATATATCCCTTTGTTGGGTT |
| | TATACCACTTAGCTTGAAAGAGGTTAAAACATTACAATTCATTGTTAAGTTG |
| | AATACAGCAAAATGGGAGTCAAAGTTCTGTTTGCCCTGATCTGCATCGCTGT |
| | GGCCGAGGCCAAGCCCACCGAGAACAACGAAGACTTCAACATCGTGGCCGTG |
| | GCCAGCAACTTCGCGACCACGGATCTCGATGCTGACCGCGGGAAGTTGCCCG |
| | GCAAGAAGCTGCCGCTGGAGGTGCTCAAAGAGATGGAAGCCAATGCCCGGAA |
| | AGCTGGCTGCACCAGGGGCTGTCTGATCTGCCTGTCCCACATCAAGTGCACG |
| | CCCAAGATGAAGAAGTTCATCCCAGGACGCTGCCACACCTACGAAGGCGACA |
| | AAGAGTCCGCACAGGGGGGCATAGGCGAGGCGATCGTCGACATTCCTGAGAT |
| | TCCTGGGTTCAAGGACTTGGAGCCCATGGAGCAGTTCATCGCACAGGTCGAT |
| | CTGTGTGTGGACTGCACAACTGGCTGCCTCAAAGGGCTTGCCAACGTGCAGT |
| | GTTCTGACCTGCTCAAGAAGTGGCTGCCGCAACGCTGTGCGACCTTTGCCAG |
| | CAAGATCCAGGGCCAGGTGGACAAGATCAAGGGGGCCGGTGGTGACTAAGTA |
| | GAAAATGCGGCCGCCGACCAGAATCATGCAAGTGCGTAAGATAGTCGCGGGT |
| | CGGCGGCCGCATCTGCTGGGAAGCTACGATCCGTAGAAAATGCGGCCGCCGA |
| | CCAGAATCATGCAAGTGCGTAAGATAGTCGCGGGTCGGCGGCCGCATCTGCT |
| | GGGTAGCTGTGAACCGTAGAAAATGCGGCCGCCGACCAGAATCATGCAAGTG |
| | CGTAAGATAGTCGCGGGTCGGCGGCCGCATCTGCTGGGAAGCTACGATCCGT |
| | AGAAAATGCGGCCGCCGACCAGAATCATGCAAGTGCGTAAGATAGTCGCGGG |
| | TCGGCGGCCGCATCTGCTGGGAGCTCGCTTTCTTGCTGTCCAATTTCTATTA |
| | AAGGTTCCTTTGTTCCCTAAGTCCAACTACTAAACTGGGGGATATTATGAAG |
| | GGCCTTGAGCATCTGGATTCTGCCTAATAAAAAACATTTATTTTCATTGCAG |
| | CTCGCTTTCTTGCTGTCCAATTTCTATTAAAGGTTCCTTTGTTCCCTAAGTC |
| | CAACTACTAAACTGGGGGATATTATGAAGGGCCTTGAGCATCTGGATTCTGC |
| | CTAATAAAAAACATTTATTTTCATTGCAAAAAACAAAAAACAAAACGGCTAT |
| | TATGCGTTACCGGCGAGACGCTACGGACTTAAATAATTGAGCCTTAAAGAAG |
| | AAATTCTTTAAGTGGATGCTCTCAAACTCAGGGAAACCTAAATCTAGTTATA |
| | GACAAGGCAATCCTGAGCCAAGCCGAAGTAGTAATTAGTAAGACCAGTGGAC |
| | AATCGACG |
| SEQ ID NO. | TAATACGACTCACTATAGGGGATCCGGGAGACCCTCGACCGTCGATTGTCCA |
| 12: | CTGGTCAACAATAGATGACTTACAACTAATCGGAAGGTGCAGAGACTCGACG |
| pML34_4xS1 | GGAGCTACCCTAACGTCAAGACGAGGGTAAAGAGAGAGTCCAATTCTCAAAG |
| m-CVB3 | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | GTAGAAAATGCGGCCGCCGACCAGAATCATGCAAGTGCGTAAGATAGTCGCG |
| | GGTCGGCGGCCGCATCTGCTGGGAAGCTACGATCCGTAGAAAATGCGGCCGC |
| | CGACCAGAATCATGCAAGTGCGTAAGATAGTCGCGGGTCGGCGGCCGCATCT |
| | GCTGGGTAGCTGTGAACCGTAGAAAATGCGGCCGCCGACCAGAATCATGCAA |
| | GTGCGTAAGATAGTCGCGGGTCGGCGGCCGCATCTGCTGGGAAGCTACGATC |
| | CGTAGAAAATGCGGCCGCCGACCAGAATCATGCAAGTGCGTAAGATAGTCGC |
| | GGGTCGGCGGCCGCATCTGCTGGGAAAAACAAAAAAAAAAAAAACAAAAAAA |
| | AAACCAAAAAAACAAAACACATTAAAACAGCCTGTGGGTTGATCCCACCCAC |
| | AGGCCCATTGGGCGCTAGCACTCTGGTATCACGGTACCTTTGTGCGCCTGTT |
| | TTATACCCCCTCCCCCAACTGTAACTTAGAAGTAACACACACCGATCAACAG |
| | TCAGCGTGGCACACCAGCCACGTTTTGATCAAGCACTTCTGTTACCCCGGAC |
| | TGAGTATCAATAGACTGCTCACGCGGTTGAAGGAGAAAGCGTTCGTTATCCG |
| | GCCAACTACTTCGAAAAACCTAGTAACACCGTGGAAGTTGCAGAGTGTTTCG |
| | CTCAGCACTACCCCAGTGTAGATCAGGTCGATGAGTCACCGCATTCCCCACG |
| | GGCGACCGTGGCGGTGGCTGCGTTGGCGGCCTGCCCATGGGGAAACCCATGG |
| | GACGCTCTAATACAGACATGGTGCGAAGAGTCTATTGAGCTAGTTGGTAGTC |
| | CTCCGGCCCCTGAATGCGGCTAATCCTAACTGCGGAGCACACACCCTCAAGC |
| | CAGAGGGCAGTGTGTCGTAACGGGCAACTCTGCAGCGGAACCGACTACTTTG |
| | GGTGTCCGTGTTTCATTTTATTCCTATACTGGCTGCTTATGGTGACAATTGA |
| | GAGATCGTTACCATATAGCTATTGGATTGGCCATCCGGTGACTAATAGAGCT |
| | ATTATATATCCCTTTGTTGGGTTTATACCACTTAGCTTGAAAGAGGTTAAAA |
| | CATTACAATTCATTGTTAAGTTGAATACAGCAAAATGGTGAGCAAGGGCGAG |
| | GAGCTGTTCACCGGGGTGGTGCCCATCCTGGTCGAGCTGGACGGCGACGTAA |
| | ACGGCCACAAGTTCAGCGTGTCTGGCGAGGGCGAGGGCGATGCCACCTACGG |
| | CAAGCTGACCCTGAAGTTCATCTGCACCACCGGCAAGCTGCCCGTGCCCTGG |
| | CCCACCCTCGTGACCACCCTGACCTACGGCGTGCAGTGCTTCAGCCGCTACC |
| | CCGACCACATGAAGCAGCACGACTTCTTCAAGTCCGCCATGCCCGAAGGCTA |
| | CGTCCAGGAGCGCACCATCTTCTTCAAGGACGACGGCAACTACAAGACCCGC |
| | GCCGAGGTGAAGTTCGAGGGCGACACCCTGGTGAACCGCATCGAGCTGAAGG |
| | GCATCGACTTCAAGGAGGACGGCAACATCCTGGGGCACAAGCTGGAGTACAA |
| | CTACAACAGCCACAACGTCTATATCATGGCCGACAAGCAGAAGAACGGCATC |
| | AAGGCGAACTTCAAGATCCGCCACAACATCGAGGACGGCAGCGTGCAGCTCG |
| | CCGACCACTACCAGCAGAACACCCCCATCGGCGACGGCCCCGTGCTGCTGCC |
| | CGACAACCACTACCTGAGCACCCAGTCCGCCCTGAGCAAAGACCCCAACGAG |
| | AAGCGCGATCACATGGTCCTGCTGGAGTTCGTGACCGCCGCCGGGATCACTC |
| | TCGGCATGGACGAGCTGTACAAGTAAAAAAAACAAAAAACAAAACGGCTATT |
| | ATGCGTTACCGGCGAGACGCTACGGACTTAAATAATTGAGCCTTAAAGAAGA |
| | AATTCTTTAAGTGGATGCTCTCAAACTCAGGGAAACCTAAATCTAGTTATAG |
| | ACAAGGCAATCCTGAGCCAAGCCGAAGTAGTAATTAGTAAGACCAGTGGACA |
| | ATCGACG |
| SEQ ID NO. | TAATACGACTCACTATAGGGGATCCGGGAGACCCTCGACCGTCGATTGTCCA |
| 13: | CTGGTCAACAATAGATGACTTACAACTAATCGGAAGGTGCAGAGACTCGACG |
| pML35_CVB | GGAGCTACCCTAACGTCAAGACGAGGGTAAAGAGAGAGTCCAATTCTCAAAG |
| 3-EGFP- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAACAAAACACATT |
| | AAAACAGCCTGTGGGTTGATCCCACCCACAGGCCCATTGGGCGCTAGCACTC |
| | TGGTATCACGGTACCTTTGTGCGCCTGTTTTATACCCCCTCCCCCAACTGTA |
| | ACTTAGAAGTAACACACACCGATCAACAGTCAGCGTGGCACACCAGCCACGT |
| | TTTGATCAAGCACTTCTGTTACCCCGGACTGAGTATCAATAGACTGCTCACG |
| | CGGTTGAAGGAGAAAGCGTTCGTTATCCGGCCAACTACTTCGAAAAACCTAG |
| | TAACACCGTGGAAGTTGCAGAGTGTTTCGCTCAGCACTACCCCAGTGTAGAT |
| | CAGGTCGATGAGTCACCGCATTCCCCACGGGCGACCGTGGCGGTGGCTGCGT |
| | TGGCGGCCTGCCCATGGGGAAACCCATGGGACGCTCTAATACAGACATGGTG |
| | CGAAGAGTCTATTGAGCTAGTTGGTAGTCCTCCGGCCCCTGAATGCGGCTAA |
| | TCCTAACTGCGGAGCACACACCCTCAAGCCAGAGGGCAGTGTGTCGTAACGG |
| | GCAACTCTGCAGCGGAACCGACTACTTTGGGTGTCCGTGTTTCATTTTATTC |
| | CTATACTGGCTGCTTATGGTGACAATTGAGAGATCGTTACCATATAGCTATT |
| | GGATTGGCCATCCGGTGACTAATAGAGCTATTATATATCCCTTTGTTGGGTT |
| | TATACCACTTAGCTTGAAAGAGGTTAAAACATTACAATTCATTGTTAAGTTG |
| | AATACAGCAAAATGGTGAGCAAGGGCGAGGAGCTGTTCACCGGGGTGGTGCC |
| | CATCCTGGTCGAGCTGGACGGCGACGTAAACGGCCACAAGTTCAGCGTGTCT |
| | GGCGAGGGCGAGGGCGATGCCACCTACGGCAAGCTGACCCTGAAGTTCATCT |
| | GCACCACCGGCAAGCTGCCCGTGCCCTGGCCCACCCTCGTGACCACCCTGAC |
| | CTACGGCGTGCAGTGCTTCAGCCGCTACCCCGACCACATGAAGCAGCACGAC |
| | TTCTTCAAGTCCGCCATGCCCGAAGGCTACGTCCAGGAGCGCACCATCTTCT |
| | TCAAGGACGACGGCAACTACAAGACCCGCGCCGAGGTGAAGTTCGAGGGCGA |
| | CACCCTGGTGAACCGCATCGAGCTGAAGGGCATCGACTTCAAGGAGGACGGC |
| | AACATCCTGGGGCACAAGCTGGAGTACAACTACAACAGCCACAACGTCTATA |
| | TCATGGCCGACAAGCAGAAGAACGGCATCAAGGCGAACTTCAAGATCCGCCA |
| | CAACATCGAGGACGGCAGCGTGCAGCTCGCCGACCACTACCAGCAGAACACC |
| | CCCATCGGCGACGGCCCCGTGCTGCTGCCCGACAACCACTACCTGAGCACCC |
| | AGTCCGCCCTGAGCAAAGACCCCAACGAGAAGCGCGATCACATGGTCCTGCT |
| | GGAGTTCGTGACCGCCGCCGGGATCACTCTCGGCATGGACGAGCTGTACAAG |
| | TAAAAAAAACAAAAAACAAAACAAAAAAAAAAAAAGCCCGGATAGCTCAGTC |
| | GGTAGAGCAGCGGCCTATGCGGCCGCCGACCAGAATCATGCAAGTGCGTAAG |
| | ATAGTCGCGGGTCGGCGGCCGCATTCGAGGCCGCGTCCAGGGTTCAAGTCCC |
| | TGTTCGGGCGCCACTGCAGAAAAAAAAAAAAGGCTATTATGCGTTACCGGCG |
| | AGACGCTACGGACTTAAATAATTGAGCCTTAAAGAAGAAATTCTTTAAGTGG |
| | ATGCTCTCAAACTCAGGGAAACCTAAATCTAGTTATAGACAAGGCAATCCTG |
| | AGCCAAGCCGAAGTAGTAATTAGTAAGACCAGTGGACAATCGACG |
| SEQ ID NO. | TAATACGACTCACTATAGGGGATCCGGGAGACCCTCGACCGTCGATTGTCCA |
| 14: | CTGGTCAACAATAGATGACTTACAACTAATCGGAAGGTGCAGAGACTCGACG |
| pML36_CVB | GGAGCTACCCTAACGTCAAGACGAGGGTAAAGAGAGAGTCCAATTCTCAAAG |
| 3-EGFP- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAACAAAACACATT |
| | AAAACAGCCTGTGGGTTGATCCCACCCACAGGCCCATTGGGCGCTAGCACTC |
| | TGGTATCACGGTACCTTTGTGCGCCTGTTTTATACCCCCTCCCCCAACTGTA |
| | ACTTAGAAGTAACACACACCGATCAACAGTCAGCGTGGCACACCAGCCACGT |
| | TTTGATCAAGCACTTCTGTTACCCCGGACTGAGTATCAATAGACTGCTCACG |
| | CGGTTGAAGGAGAAAGCGTTCGTTATCCGGCCAACTACTTCGAAAAACCTAG |
| | TAACACCGTGGAAGTTGCAGAGTGTTTCGCTCAGCACTACCCCAGTGTAGAT |
| | CAGGTCGATGAGTCACCGCATTCCCCACGGGCGACCGTGGCGGTGGCTGCGT |
| | TGGCGGCCTGCCCATGGGGAAACCCATGGGACGCTCTAATACAGACATGGTG |
| | CGAAGAGTCTATTGAGCTAGTTGGTAGTCCTCCGGCCCCTGAATGCGGCTAA |
| | TCCTAACTGCGGAGCACACACCCTCAAGCCAGAGGGCAGTGTGTCGTAACGG |
| | GCAACTCTGCAGCGGAACCGACTACTTTGGGTGTCCGTGTTTCATTTTATTC |
| | CTATACTGGCTGCTTATGGTGACAATTGAGAGATCGTTACCATATAGCTATT |
| | GGATTGGCCATCCGGTGACTAATAGAGCTATTATATATCCCTTTGTTGGGTT |
| | TATACCACTTAGCTTGAAAGAGGTTAAAACATTACAATTCATTGTTAAGTTG |
| | AATACAGCAAAATGGTGAGCAAGGGCGAGGAGCTGTTCACCGGGGTGGTGCC |
| | CATCCTGGTCGAGCTGGACGGCGACGTAAACGGCCACAAGTTCAGCGTGTCT |
| | GGCGAGGGCGAGGGCGATGCCACCTACGGCAAGCTGACCCTGAAGTTCATCT |
| | GCACCACCGGCAAGCTGCCCGTGCCCTGGCCCACCCTCGTGACCACCCTGAC |
| | CTACGGCGTGCAGTGCTTCAGCCGCTACCCCGACCACATGAAGCAGCACGAC |
| | TTCTTCAAGTCCGCCATGCCCGAAGGCTACGTCCAGGAGCGCACCATCTTCT |
| | TCAAGGACGACGGCAACTACAAGACCCGCGCCGAGGTGAAGTTCGAGGGCGA |
| | CACCCTGGTGAACCGCATCGAGCTGAAGGGCATCGACTTCAAGGAGGACGGC |
| | AACATCCTGGGGCACAAGCTGGAGTACAACTACAACAGCCACAACGTCTATA |
| | TCATGGCCGACAAGCAGAAGAACGGCATCAAGGCGAACTTCAAGATCCGCCA |
| | CAACATCGAGGACGGCAGCGTGCAGCTCGCCGACCACTACCAGCAGAACACC |
| | CCCATCGGCGACGGCCCCGTGCTGCTGCCCGACAACCACTACCTGAGCACCC |
| | AGTCCGCCCTGAGCAAAGACCCCAACGAGAAGCGCGATCACATGGTCCTGCT |
| | GGAGTTCGTGACCGCCGCCGGGATCACTCTCGGCATGGACGAGCTGTACAAG |
| | TAAAAAAAACAAAAAACAAAACAAAAAAAAAAAAAGCCCGGATAGCTCAGTC |
| | GGTAGAGCAGCGGCCTATGCGGCCGCCGACCAGAATCATGCAAGTGCGTAAG |
| | ATAGTCGCGGGTCGGCGGCCGCATTCGAGGCCGCGTCCAGGGTTCAAGTCCC |
| | TGTTCGGGCGCCACTGCAGAAAAAAAAAAAAAGCTCGCTTTCTTGCTGTCCA |
| | ATTTCTATTAAAGGTTCCTTTGTTCCCTAAGTCCAACTACTAAACTGGGGGA |
| | TATTATGAAGGGCCTTGAGCATCTGGATTCTGCCTAATAAAAAACATTTATT |
| | TTCATTGCAGCTCGCTTTCTTGCTGTCCAATTTCTATTAAAGGTTCCTTTGT |
| | TCCCTAAGTCCAACTACTAAACTGGGGGATATTATGAAGGGCCTTGAGCATC |
| | TGGATTCTGCCTAATAAAAAACATTTATTTTCATTGCGGCTATTATGCGTTA |
| | CCGGCGAGACGCTACGGACTTAAATAATTGAGCCTTAAAGAAGAAATTCTTT |
| | AAGTGGATGCTCTCAAACTCAGGGAAACCTAAATCTAGTTATAGACAAGGCA |
| | ATCCTGAGCCAAGCCGAAGTAGTAATTAGTAAGACCAGTGGACAATCGACG |
| SEQ ID NO. | TAATACGACTCACTATAGGGGATCCAGAGCGGCCGCTTTTTCAGCAAGATTA |
| 15: | AGCCCAGGGCAGAGCCATCTATTGOTTACATTTGCTTCTGACACAACTGTGT |
| pML37_UTR- | TCACTAGCAACCTCAAACAGACACCGGGAGACCCTCGACCGTCGATTGTCCA |
| CVB3-E | CTGGTCAACAATAGATGACTTACAACTAATCGGAAGGTGCAGAGACTCGACG |
| | GGAGCTACCCTAACGTCAAGACGAGGGTAAAGAGAGAGTCCAATTCTCAAAG |
| | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAACAAAACACATT |
| | AAAACAGCCTGTGGGTTGATCCCACCCACAGGCCCATTGGGCGCTAGCACTC |
| | TGGTATCACGGTACCTTTGTGCGCCTGTTTTATACCCCCTCCCCCAACTGTA |
| | ACTTAGAAGTAACACACACCGATCAACAGTCAGCGTGGCACACCAGCCACGT |
| | TTTGATCAAGCACTTCTGTTACCCCGGACTGAGTATCAATAGACTGCTCACG |
| | CGGTTGAAGGAGAAAGCGTTCGTTATCCGGCCAACTACTTCGAAAAACCTAG |
| | TAACACCGTGGAAGTTGCAGAGTGTTTCGCTCAGCACTACCCCAGTGTAGAT |
| | CAGGTCGATGAGTCACCGCATTCCCCACGGGCGACCGTGGCGGTGGCTGCGT |
| | TGGCGGCCTGCCCATGGGGAAACCCATGGGACGCTCTAATACAGACATGGTG |
| | CGAAGAGTCTATTGAGCTAGTTGGTAGTCCTCCGGCCCCTGAATGCGGCTAA |
| | TCCTAACTGCGGAGCACACACCCTCAAGCCAGAGGGCAGTGTGTCGTAACGG |
| | GCAACTCTGCAGCGGAACCGACTACTTTGGGTGTCCGTGTTTCATTTTATTC |
| | CTATACTGGCTGCTTATGGTGACAATTGAGAGATCGTTACCATATAGCTATT |
| | GGATTGGCCATCCGGTGACTAATAGAGCTATTATATATCCCTTTGTTGGGTT |
| | TATACCACTTAGCTTGAAAGAGGTTAAAACATTACAATTCATTGTTAAGTTG |
| | AATACAGCAAAATGGTGAGCAAGGGCGAGGAGCTGTTCACCGGGGTGGTGCC |
| | CATCCTGGTCGAGCTGGACGGCGACGTAAACGGCCACAAGTTCAGCGTGTCT |
| | GGCGAGGGCGAGGGCGATGCCACCTACGGCAAGCTGACCCTGAAGTTCATCT |
| | GCACCACCGGCAAGCTGCCCGTGCCCTGGCCCACCCTCGTGACCACCCTGAC |
| | CTACGGCGTGCAGTGCTTCAGCCGCTACCCCGACCACATGAAGCAGCACGAC |
| | TTCTTCAAGTCCGCCATGCCCGAAGGCTACGTCCAGGAGCGCACCATCTTCT |
| | TCAAGGACGACGGCAACTACAAGACCCGCGCCGAGGTGAAGTTCGAGGGCGA |
| | CACCCTGGTGAACCGCATCGAGCTGAAGGGCATCGACTTCAAGGAGGACGGC |
| | AACATCCTGGGGCACAAGCTGGAGTACAACTACAACAGCCACAACGTCTATA |
| | TCATGGCCGACAAGCAGAAGAACGGCATCAAGGCGAACTTCAAGATCCGCCA |
| | CAACATCGAGGACGGCAGCGTGCAGCTCGCCGACCACTACCAGCAGAACACC |
| | CCCATCGGCGACGGCCCCGTGCTGCTGCCCGACAACCACTACCTGAGCACCC |
| | AGTCCGCCCTGAGCAAAGACCCCAACGAGAAGCGCGATCACATGGTCCTGCT |
| | GGAGTTCGTGACCGCCGCCGGGATCACTCTCGGCATGGACGAGCTGTACAAG |
| | TAAAAAAAACAAAAAACAAAACAAAAAAAAAAAAAGCCCGGATAGCTCAGTC |
| | GGTAGAGCAGCGGCCTATGCGGCCGCCGACCAGAATCATGCAAGTGCGTAAG |
| | ATAGTCGCGGGTCGGCGGCCGCATTCGAGGCCGCGTCCAGGGTTCAAGTCCC |
| | TGTTCGGGCGCCACTGCAGAAAAAAAAAAAAAGCTCGCTTTCTTGCTGTCCA |
| | ATTTCTATTAAAGGTTCCTTTGTTCCCTAAGTCCAACTACTAAACTGGGGGA |
| | TATTATGAAGGGCCTTGAGCATCTGGATTCTGCCTAATAAAAAACATTTATT |
| | TTCATTGCAGCTCGCTTTCTTGCTGTCCAATTTCTATTAAAGGTTCCTTTGT |
| | TCCCTAAGTCCAACTACTAAACTGGGGGATATTATGAAGGGCCTTGAGCATC |
| | TGGATTCTGCCTAATAAAAAACATTTATTTTCATTGCGGCTATTATGCGTTA |
| | CCGGCGAGACGCTACGGACTTAAATAATTGAGCCTTAAAGAAGAAATTCTTT |
| | AAGTGGATGCTCTCAAACTCAGGGAAACCTAAATCTAGTTATAGACAAGGCA |
| | ATCCTGAGCCAAGCCGAAGTAGTAATTAGTAAGACCAGTGGACAATCGACG |
| SEQ ID NO. | TAATACGACTCACTATAGGGGATCCGGGAGACCCTCGACCGTCGATTGTCCA |
| 16: | CTGGTCAACAATAGATGACTTACAACTAATCGGAAGGTGCAGAGACTCGACG |
| pML38_CVB | GGAGCTACCCTAACGTCAAGACGAGGGTAAAGAGAGAGTCCAATTCTCAAAG |
| 3-EGFP- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAACAAAACACATT |
| | AAAACAGCCTGTGGGTTGATCCCACCCACAGGCCCATTGGGCGCTAGCACTC |
| | TGGTATCACGGTACCTTTGTGCGCCTGTTTTATACCCCCTCCCCCAACTGTA |
| | ACTTAGAAGTAACACACACCGATCAACAGTCAGCGTGGCACACCAGCCACGT |
| | TTTGATCAAGCACTTCTGTTACCCCGGACTGAGTATCAATAGACTGCTCACG |
| | CGGTTGAAGGAGAAAGCGTTCGTTATCCGGCCAACTACTTCGAAAAACCTAG |
| | TAACACCGTGGAAGTTGCAGAGTGTTTCGCTCAGCACTACCCCAGTGTAGAT |
| | CAGGTCGATGAGTCACCGCATTCCCCACGGGCGACCGTGGCGGTGGCTGCGT |
| | TGGCGGCCTGCCCATGGGGAAACCCATGGGACGCTCTAATACAGACATGGTG |
| | CGAAGAGTCTATTGAGCTAGTTGGTAGTCCTCCGGCCCCTGAATGCGGCTAA |
| | TCCTAACTGCGGAGCACACACCCTCAAGCCAGAGGGCAGTGTGTCGTAACGG |
| | GCAACTCTGCAGCGGAACCGACTACTTTGGGTGTCCGTGTTTCATTTTATTC |
| | CTATACTGGCTGCTTATGGTGACAATTGAGAGATCGTTACCATATAGCTATT |
| | GGATTGGCCATCCGGTGACTAATAGAGCTATTATATATCCCTTTGTTGGGTT |
| | TATACCACTTAGCTTGAAAGAGGTTAAAACATTACAATTCATTGTTAAGTTG |
| | AATACAGCAAAATGGTGAGCAAGGGCGAGGAGCTGTTCACCGGGGTGGTGCC |
| | CATCCTGGTCGAGCTGGACGGCGACGTAAACGGCCACAAGTTCAGCGTGTCT |
| | GGCGAGGGCGAGGGCGATGCCACCTACGGCAAGCTGACCCTGAAGTTCATCT |
| | GCACCACCGGCAAGCTGCCCGTGCCCTGGCCCACCCTCGTGACCACCCTGAC |
| | CTACGGCGTGCAGTGCTTCAGCCGCTACCCCGACCACATGAAGCAGCACGAC |
| | TTCTTCAAGTCCGCCATGCCCGAAGGCTACGTCCAGGAGCGCACCATCTTCT |
| | TCAAGGACGACGGCAACTACAAGACCCGCGCCGAGGTGAAGTTCGAGGGCGA |
| | CACCCTGGTGAACCGCATCGAGCTGAAGGGCATCGACTTCAAGGAGGACGGC |
| | AACATCCTGGGGCACAAGCTGGAGTACAACTACAACAGCCACAACGTCTATA |
| | TCATGGCCGACAAGCAGAAGAACGGCATCAAGGCGAACTTCAAGATCCGCCA |
| | CAACATCGAGGACGGCAGCGTGCAGCTCGCCGACCACTACCAGCAGAACACC |
| | CCCATCGGCGACGGCCCCGTGCTGCTGCCCGACAACCACTACCTGAGCACCC |
| | AGTCCGCCCTGAGCAAAGACCCCAACGAGAAGCGCGATCACATGGTCCTGCT |
| | GGAGTTCGTGACCGCCGCCGGGATCACTCTCGGCATGGACGAGCTGTACAAG |
| | TAAAAAAAAAAAAAAAGCCCGGATAGCTCAGTCGGTAGAGCAGCGGCCTATG |
| | CGGCCGCCGACCAGAATCATGCAAGTGCGTAAGATAGTCGCGGGTCGGCGGC |
| | CGCATTCGAGGCCGCGTCCAGGGTTCAAGTCCCTGTTCGGGCGCCACTGCAG |
| | AAAAAAAAAAAAAAAAAACAAAAAACAAAACGGCTATTATGCGTTACCGGCG |
| | AGACGCTACGGACTTAAATAATTGAGCCTTAAAGAAGAAATTCTTTAAGTGG |
| | ATGCTCTCAAACTCAGGGAAACCTAAATCTAGTTATAGACAAGGCAATCCTG |
| | AGCCAAGCCGAAGTAGTAATTAGTAAGACCAGTGGACAATCGACG |
| SEQ ID NO. | TAATACGACTCACTATAGGGGATCCGGGAGACCCTCGACCGTCGATTGTCCA |
| 17: | CTGGTCAACAATAGATGACTTACAACTAATCGGAAGGTGCAGAGACTCGACG |
| pML39_CVB | GGAGCTACCCTAACGTCAAGACGAGGGTAAAGAGAGAGTCCAATTCTCAAAG |
| 3-EGFP- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAACAAAACACATT |
| | AAAACAGCCTGTGGGTTGATCCCACCCACAGGCCCATTGGGCGCTAGCACTC |
| | TGGTATCACGGTACCTTTGTGCGCCTGTTTTATACCCCCTCCCCCAACTGTA |
| | ACTTAGAAGTAACACACACCGATCAACAGTCAGCGTGGCACACCAGCCACGT |
| | TTTGATCAAGCACTTCTGTTACCCCGGACTGAGTATCAATAGACTGCTCACG |
| | CGGTTGAAGGAGAAAGCGTTCGTTATCCGGCCAACTACTTCGAAAAACCTAG |
| | TAACACCGTGGAAGTTGCAGAGTGTTTCGCTCAGCACTACCCCAGTGTAGAT |
| | CAGGTCGATGAGTCACCGCATTCCCCACGGGCGACCGTGGCGGTGGCTGCGT |
| | TGGCGGCCTGCCCATGGGGAAACCCATGGGACGCTCTAATACAGACATGGTG |
| | CGAAGAGTCTATTGAGCTAGTTGGTAGTCCTCCGGCCCCTGAATGCGGCTAA |
| | TCCTAACTGCGGAGCACACACCCTCAAGCCAGAGGGCAGTGTGTCGTAACGG |
| | GCAACTCTGCAGCGGAACCGACTACTTTGGGTGTCCGTGTTTCATTTTATTC |
| | CTATACTGGCTGCTTATGGTGACAATTGAGAGATCGTTACCATATAGCTATT |
| | GGATTGGCCATCCGGTGACTAATAGAGCTATTATATATCCCTTTGTTGGGTT |
| | TATACCACTTAGCTTGAAAGAGGTTAAAACATTACAATTCATTGTTAAGTTG |
| | AATACAGCAAAATGGTGAGCAAGGGCGAGGAGCTGTTCACCGGGGTGGTGCC |
| | CATCCTGGTCGAGCTGGACGGCGACGTAAACGGCCACAAGTTCAGCGTGTCT |
| | GGCGAGGGCGAGGGCGATGCCACCTACGGCAAGCTGACCCTGAAGTTCATCT |
| | GCACCACCGGCAAGCTGCCCGTGCCCTGGCCCACCCTCGTGACCACCCTGAC |
| | CTACGGCGTGCAGTGCTTCAGCCGCTACCCCGACCACATGAAGCAGCACGAC |
| | TTCTTCAAGTCCGCCATGCCCGAAGGCTACGTCCAGGAGCGCACCATCTTCT |
| | TCAAGGACGACGGCAACTACAAGACCCGCGCCGAGGTGAAGTTCGAGGGCGA |
| | CACCCTGGTGAACCGCATCGAGCTGAAGGGCATCGACTTCAAGGAGGACGGC |
| | AACATCCTGGGGCACAAGCTGGAGTACAACTACAACAGCCACAACGTCTATA |
| | TCATGGCCGACAAGCAGAAGAACGGCATCAAGGCGAACTTCAAGATCCGCCA |
| | CAACATCGAGGACGGCAGCGTGCAGCTCGCCGACCACTACCAGCAGAACACC |
| | CCCATCGGCGACGGCCCCGTGCTGCTGCCCGACAACCACTACCTGAGCACCC |
| | AGTCCGCCCTGAGCAAAGACCCCAACGAGAAGCGCGATCACATGGTCCTGCT |
| | GGAGTTCGTGACCGCCGCCGGGATCACTCTCGGCATGGACGAGCTGTACAAG |
| | TAAAGCTCGCTTTCTTGCTGTCCAATTTCTATTAAAGGTTCCTTTGTTCCCT |
| | AAGTCCAACTACTAAACTGGGGGATATTATGAAGGGCCTTGAGCATCTGGAT |
| | TCTGCCTAATAAAAAACATTTATTTTCATTGCAGCTCGCTTTCTTGCTGTCC |
| | AATTTCTATTAAAGGTTCCTTTGTTCCCTAAGTCCAACTACTAAACTGGGGG |
| | ATATTATGAAGGGCCTTGAGCATCTGGATTCTGCCTAATAAAAAACATTTAT |
| | TTTCATTGCAAAAAAAAAAAAAGCCCGGATAGCTCAGTCGGTAGAGCAGCGG |
| | CCTATGCGGCCGCCGACCAGAATCATGCAAGTGCGTAAGATAGTCGCGGGTC |
| | GGCGGCCGCATTCGAGGCCGCGTCCAGGGTTCAAGTCCCTGTTCGGGCGCCA |
| | CTGCAGAAAAAAAAAAAAAAAAAACAAAAAACAAAACGGCTATTATGCGTTA |
| | CCGGCGAGACGCTACGGACTTAAATAATTGAGCCTTAAAGAAGAAATTCTTT |
| | AAGTGGATGCTCTCAAACTCAGGGAAACCTAAATCTAGTTATAGACAAGGCA |
| | ATCCTGAGCCAAGCCGAAGTAGTAATTAGTAAGACCAGTGGACAATCGACG |
| SEQ ID NO. | TAATACGACTCACTATAGGGGATCCGGGAGACCCTCGACCGTCGATTGTCCA |
| 18: | CTGGTCAACAATAGATGACTTACAACTAATCGGAAGGTGCAGAGACTCGACG |
| pML40_CVB | GGAGCTACCCTAACGTCAAGACGAGGGTAAAGAGAGAGTCCAATTCTCAAAG |
| 3-EGFP- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAACAAAACACATT |
| | AAAACAGCCTGTGGGTTGATCCCACCCACAGGCCCATTGGGCGCTAGCACTC |
| | TGGTATCACGGTACCTTTGTGCGCCTGTTTTATACCCCCTCCCCCAACTGTA |
| | ACTTAGAAGTAACACACACCGATCAACAGTCAGCGTGGCACACCAGCCACGT |
| | TTTGATCAAGCACTTCTGTTACCCCGGACTGAGTATCAATAGACTGCTCACG |
| | CGGTTGAAGGAGAAAGCGTTCGTTATCCGGCCAACTACTTCGAAAAACCTAG |
| | TAACACCGTGGAAGTTGCAGAGTGTTTCGCTCAGCACTACCCCAGTGTAGAT |
| | CAGGTCGATGAGTCACCGCATTCCCCACGGGCGACCGTGGCGGTGGCTGCGT |
| | TGGCGGCCTGCCCATGGGGAAACCCATGGGACGCTCTAATACAGACATGGTG |
| | CGAAGAGTCTATTGAGCTAGTTGGTAGTCCTCCGGCCCCTGAATGCGGCTAA |
| | TCCTAACTGCGGAGCACACACCCTCAAGCCAGAGGGCAGTGTGTCGTAACGG |
| | GCAACTCTGCAGCGGAACCGACTACTTTGGGTGTCCGTGTTTCATTTTATTC |
| | CTATACTGGCTGCTTATGGTGACAATTGAGAGATCGTTACCATATAGCTATT |
| | GGATTGGCCATCCGGTGACTAATAGAGCTATTATATATCCCTTTGTTGGGTT |
| | TATACCACTTAGCTTGAAAGAGGTTAAAACATTACAATTCATTGTTAAGTTG |
| | AATACAGCAAAATGGTGAGCAAGGGCGAGGAGCTGTTCACCGGGGTGGTGCC |
| | CATCCTGGTCGAGCTGGACGGCGACGTAAACGGCCACAAGTTCAGCGTGTCT |
| | GGCGAGGGCGAGGGCGATGCCACCTACGGCAAGCTGACCCTGAAGTTCATCT |
| | GCACCACCGGCAAGCTGCCCGTGCCCTGGCCCACCCTCGTGACCACCCTGAC |
| | CTACGGCGTGCAGTGCTTCAGCCGCTACCCCGACCACATGAAGCAGCACGAC |
| | TTCTTCAAGTCCGCCATGCCCGAAGGCTACGTCCAGGAGCGCACCATCTTCT |
| | TCAAGGACGACGGCAACTACAAGACCCGCGCCGAGGTGAAGTTCGAGGGCGA |
| | CACCCTGGTGAACCGCATCGAGCTGAAGGGCATCGACTTCAAGGAGGACGGC |
| | AACATCCTGGGGCACAAGCTGGAGTACAACTACAACAGCCACAACGTCTATA |
| | TCATGGCCGACAAGCAGAAGAACGGCATCAAGGCGAACTTCAAGATCCGCCA |
| | CAACATCGAGGACGGCAGCGTGCAGCTCGCCGACCACTACCAGCAGAACACC |
| | CCCATCGGCGACGGCCCCGTGCTGCTGCCCGACAACCACTACCTGAGCACCC |
| | AGTCCGCCCTGAGCAAAGACCCCAACGAGAAGCGCGATCACATGGTCCTGCT |
| | GGAGTTCGTGACCGCCGCCGGGATCACTCTCGGCATGGACGAGCTGTACAAG |
| | TAAAAAAAAAAAAAAAGCCCGGATAGCTCAGTCGGTAGAGCAGCGGCCTATG |
| | CGGCCGCCGACCAGAATCATGCAAGTGCGTAAGATAGTCGCGGGTCGGCGGC |
| | CGCATTCGAGGCCGCGTCCAGGGTTCAAGTCCCTGTTCGGGCGCCACTGCAG |
| | AAAAAAAAAAAAAGCTCGCTTTCTTGCTGTCCAATTTCTATTAAAGGTTCCT |
| | TTGTTCCCTAAGTCCAACTACTAAACTGGGGGATATTATGAAGGGCCTTGAG |
| | CATCTGGATTCTGCCTAATAAAAAACATTTATTTTCATTGCAGCTCGCTTTC |
| | TTGCTGTCCAATTTCTATTAAAGGTTCCTTTGTTCCCTAAGTCCAACTACTA |
| | AACTGGGGGATATTATGAAGGGCCTTGAGCATCTGGATTCTGCCTAATAAAA |
| | AACATTTATTTTCATTGCAAAAAACAAAAAACAAAACGGCTATTATGCGTTA |
| | CCGGCGAGACGCTACGGACTTAAATAATTGAGCCTTAAAGAAGAAATTCTTT |
| | AAGTGGATGCTCTCAAACTCAGGGAAACCTAAATCTAGTTATAGACAAGGCA |
| | ATCCTGAGCCAAGCCGAAGTAGTAATTAGTAAGACCAGTGGACAATCGACG |
| SEQ ID NO. | TAATACGACTCACTATAGGGGATCCGGGAGACCCTCGACCGTCGATTGTCCA |
| 19: | CTGGTCAACAATAGATGACTTACAACTAATCGGAAGGTGCAGAGACTCGACG |
| pML41_CVB | GGAGCTACCCTAACGTCAAGACGAGGGTAAAGAGAGAGTCCAATTCTCAAAG |
| 3-GLuc- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAACAAAACACATT |
| | AAAACAGCCTGTGGGTTGATCCCACCCACAGGCCCATTGGGCGCTAGCACTC |
| | TGGTATCACGGTACCTTTGTGCGCCTGTTTTATACCCCCTCCCCCAACTGTA |
| | ACTTAGAAGTAACACACACCGATCAACAGTCAGCGTGGCACACCAGCCACGT |
| | TTTGATCAAGCACTTCTGTTACCCCGGACTGAGTATCAATAGACTGCTCACG |
| | CGGTTGAAGGAGAAAGCGTTCGTTATCCGGCCAACTACTTCGAAAAACCTAG |
| | TAACACCGTGGAAGTTGCAGAGTGTTTCGCTCAGCACTACCCCAGTGTAGAT |
| | CAGGTCGATGAGTCACCGCATTCCCCACGGGCGACCGTGGCGGTGGCTGCGT |
| | TGGCGGCCTGCCCATGGGGAAACCCATGGGACGCTCTAATACAGACATGGTG |
| | CGAAGAGTCTATTGAGCTAGTTGGTAGTCCTCCGGCCCCTGAATGCGGCTAA |
| | TCCTAACTGCGGAGCACACACCCTCAAGCCAGAGGGCAGTGTGTCGTAACGG |
| | GCAACTCTGCAGCGGAACCGACTACTTTGGGTGTCCGTGTTTCATTTTATTC |
| | CTATACTGGCTGCTTATGGTGACAATTGAGAGATCGTTACCATATAGCTATT |
| | GGATTGGCCATCCGGTGACTAATAGAGCTATTATATATCCCTTTGTTGGGTT |
| | TATACCACTTAGCTTGAAAGAGGTTAAAACATTACAATTCATTGTTAAGTTG |
| | AATACAGCAAAATGGGAGTCAAAGTTCTGTTTGCCCTGATCTGCATCGCTGT |
| | GGCCGAGGCCAAGCCCACCGAGAACAACGAAGACTTCAACATCGTGGCCGTG |
| | GCCAGCAACTTCGCGACCACGGATCTCGATGCTGACCGCGGGAAGTTGCCCG |
| | GCAAGAAGCTGCCGCTGGAGGTGCTCAAAGAGATGGAAGCCAATGCCCGGAA |
| | AGCTGGCTGCACCAGGGGCTGTCTGATCTGCCTGTCCCACATCAAGTGCACG |
| | CCCAAGATGAAGAAGTTCATCCCAGGACGCTGCCACACCTACGAAGGCGACA |
| | AAGAGTCCGCACAGGGCGGCATAGGCGAGGCGATCGTCGACATTCCTGAGAT |
| | TCCTGGGTTCAAGGACTTGGAGCCCATGGAGCAGTTCATCGCACAGGTCGAT |
| | CTGTGTGTGGACTGCACAACTGGCTGCCTCAAAGGGCTTGCCAACGTGCAGT |
| | GTTCTGACCTGCTCAAGAAGTGGCTGCCGCAACGCTGTGCGACCTTTGCCAG |
| | CAAGATCCAGGGCCAGGTGGACAAGATCAAGGGGGCCGGTGGTGACTAAAAA |
| | AAACAAAAAACAAAACAAAAAAAAAAAAAGCCCGGATAGCTCAGTCGGTAGA |
| | GCAGCGGCCTATGCGGCCGCCGACCAGAATCATGCAAGTGCGTAAGATAGTC |
| | GCGGGTCGGCGGCCGCATTCGAGGCCGCGTCCAGGGTTCAAGTCCCTGTTCG |
| | GGCGCCACTGCAGAAAAAAAAAAAAGGCTATTATGCGTTACCGGCGAGACGC |
| | TACGGACTTAAATAATTGAGCCTTAAAGAAGAAATTCTTTAAGTGGATGCTC |
| | TCAAACTCAGGGAAACCTAAATCTAGTTATAGACAAGGCAATCCTGAGCCAA |
| | GCCGAAGTAGTAATTAGTAAGACCAGTGGACAATCGACG |
| SEQ ID NO. | TAATACGACTCACTATAGGGGATCCGGGAGACCCTCGACCGTCGATTGTCCA |
| 20: | CTGGTCAACAATAGATGACTTACAACTAATCGGAAGGTGCAGAGACTCGACG |
| pML42_CVB | GGAGCTACCCTAACGTCAAGACGAGGGTAAAGAGAGAGTCCAATTCTCAAAG |
| 3-GLuc- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAAAAAACACATTA |
| | AAACAGCCTGTGGGTTGATCCCACCCACAGGCCCATTGGGCGCTAGCACTCT |
| | GGTATCACGGTACCTTTGTGCGCCTGTTTTATACCCCCTCCCCCAACTGTAA |
| | CTTAGAAGTAACACACACCGATCAACAGTCAGCGTGGCACACCAGCCACGTT |
| | TTGATCAAGCACTTCTGTTACCCCGGACTGAGTATCAATAGACTGCTCACGC |
| | GGTTGAAGGAGAAAGCGTTCGTTATCCGGCCAACTACTTCGAAAAACCTAGT |
| | AACACCGTGGAAGTTGCAGAGTGTTTCGCTCAGCACTACCCCAGTGTAGATC |
| | AGGTCGATGAGTCACCGCATTCCCCACGGGCGACCGTGGCGGTGGCTGCGTT |
| | GGCGGCCTGCCCATGGGGAAACCCATGGGACGCTCTAATACAGACATGGTGC |
| | GAAGAGTCTATTGAGCTAGTTGGTAGTCCTCCGGCCCCTGAATGCGGCTAAT |
| | CCTAACTGCGGAGCACACACCCTCAAGCCAGAGGGCAGTGTGTCGTAACGGG |
| | CAACTCTGCAGCGGAACCGACTACTTTGGGTGTCCGTGTTTCATTTTATTCC |
| | TATACTGGCTGCTTATGGTGACAATTGAGAGATCGTTACCATATAGCTATTG |
| | GATTGGCCATCCGGTGACTAATAGAGCTATTATATATCCCTTTGTTGGGTTT |
| | ATACCACTTAGCTTGAAAGAGGTTAAAACATTACAATTCATTGTTAAGTTGA |
| | ATACAGCAAAATGGGAGTCAAAGTTCTGTTTGCCCTGATCTGCATCGCTGTG |
| | GCCGAGGCCAAGCCCACCGAGAACAACGAAGACTTCAACATCGTGGCCGTGG |
| | CCAGCAACTTCGCGACCACGGATCTCGATGCTGACCGCGGGAAGTTGCCCGG |
| | CAAGAAGCTGCCGCTGGAGGTGCTCAAAGAGATGGAAGCCAATGCCCGGAAA |
| | GCTGGCTGCACCAGGGGCTGTCTGATCTGCCTGTCCCACATCAAGTGCACGC |
| | CCAAGATGAAGAAGTTCATCCCAGGACGCTGCCACACCTACGAAGGCGACAA |
| | AGAGTCCGCACAGGGGGGCATAGGCGAGGCGATCGTCGACATTCCTGAGATT |
| | CCTGGGTTCAAGGACTTGGAGCCCATGGAGCAGTTCATCGCACAGGTCGATC |
| | TGTGTGTGGACTGCACAACTGGCTGCCTCAAAGGGCTTGCCAACGTGCAGTG |
| | TTCTGACCTGCTCAAGAAGTGGCTGCCGCAACGCTGTGCGACCTTTGCCAGC |
| | AAGATCCAGGGCCAGGTGGACAAGATCAAGGGGGCCGGTGGTGACTAAAAAA |
| | AACAAAAAACAAAACAAAAAAAAAAAAAGCCCGGATAGCTCAGTCGGTAGAG |
| | CAGCGGCCTATGCGGCCGCCGACCAGAATCATGCAAGTGCGTAAGATAGTCG |
| | CGGGTCGGCGGCCGCATTCGAGGCCGCGTCCAGGGTTCAAGTCCCTGTTCGG |
| | GCGCCACTGCAGAAAAAAAAAAAAAGCTCGCTTTCTTGCTGTCCAATTTCTA |
| | TTAAAGGTTCCTTTGTTCCCTAAGTCCAACTACTAAACTGGGGGATATTATG |
| | AAGGGCCTTGAGCATCTGGATTCTGCCTAATAAAAAACATTTATTTTCATTG |
| | CAGCTCGCTTTCTTGCTGTCCAATTTCTATTAAAGGTTCCTTTGTTCCCTAA |
| | GTCCAACTACTAAACTGGGGGATATTATGAAGGGCCTTGAGCATCTGGATTC |
| | TGCCTAATAAAAAACATTTATTTTCATTGCGGCTATTATGCGTTACCGGCGA |
| | GACGCTACGGACTTAAATAATTGAGCCTTAAAGAAGAAATTCTTTAAGTGGA |
| | TGCTCTCAAACTCAGGGAAACCTAAATCTAGTTATAGACAAGGCAATCCTGA |
| | GCCAAGCCGAAGTAGTAATTAGTAAGACCAGTGGACAATCGACG |
| SEQ ID NO. | TAATACGACTCACTATAGGGGATCCGGGAGACCCTCGACCGTCGATTGTCCA |
| 21: | CTGGTCAACAATAGATGACTTACAACTAATCGGAAGGTGCAGAGACTCGACG |
| pML43_CVB | GGAGCTACCCTAACGTCAAGACGAGGGTAAAGAGAGAGTCCAATTCTCAAAG |
| 3-GLuc- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAAAAAAAAAAAACCAAAAAAACAAAACACATTA |
| | AAACAGCCTGTGGGTTGATCCCACCCACAGGCCCATTGGGCGCTAGCACTCT |
| | GGTATCACGGTACCTTTGTGCGCCTGTTTTATACCCCCTCCCCCAACTGTAA |
| | CTTAGAAGTAACACACACCGATCAACAGTCAGCGTGGCACACCAGCCACGTT |
| | TTGATCAAGCACTTCTGTTACCCCGGACTGAGTATCAATAGACTGCTCACGC |
| | GGTTGAAGGAGAAAGCGTTCGTTATCCGGCCAACTACTTCGAAAAACCTAGT |
| | AACACCGTGGAAGTTGCAGAGTGTTTCGCTCAGCACTACCCCAGTGTAGATC |
| | AGGTCGATGAGTCACCGCATTCCCCACGGGCGACCGTGGCGGTGGCTGCGTT |
| | GGCGGCCTGCCCATGGGGAAACCCATGGGACGCTCTAATACAGACATGGTGC |
| | GAAGAGTCTATTGAGCTAGTTGGTAGTCCTCCGGCCCCTGAATGCGGCTAAT |
| | CCTAACTGCGGAGCACACACCCTCAAGCCAGAGGGCAGTGTGTCGTAACGGG |
| | CAACTCTGCAGCGGAACCGACTACTTTGGGTGTCCGTGTTTCATTTTATTCC |
| | TATACTGGCTGCTTATGGTGACAATTGAGAGATCGTTACCATATAGCTATTG |
| | GATTGGCCATCCGGTGACTAATAGAGCTATTATATATCCCTTTGTTGGGTTT |
| | ATACCACTTAGCTTGAAAGAGGTTAAAACATTACAATTCATTGTTAAGTTGA |
| | ATACAGCAAAATGGGAGTCAAAGTTCTGTTTGCCCTGATCTGCATCGCTGTG |
| | GCCGAGGCCAAGCCCACCGAGAACAACGAAGACTTCAACATCGTGGCCGTGG |
| | CCAGCAACTTCGCGACCACGGATCTCGATGCTGACCGCGGGAAGTTGCCCGG |
| | CAAGAAGCTGCCGCTGGAGGTGCTCAAAGAGATGGAAGCCAATGCCCGGAAA |
| | GCTGGCTGCACCAGGGGCTGTCTGATCTGCCTGTCCCACATCAAGTGCACGC |
| | CCAAGATGAAGAAGTTCATCCCAGGACGCTGCCACACCTACGAAGGCGACAA |
| | AGAGTCCGCACAGGGGGGCATAGGCGAGGCGATCGTCGACATTCCTGAGATT |
| | CCTGGGTTCAAGGACTTGGAGCCCATGGAGCAGTTCATCGCACAGGTCGATC |
| | TGTGTGTGGACTGCACAACTGGCTGCCTCAAAGGGCTTGCCAACGTGCAGTG |
| | TTCTGACCTGCTCAAGAAGTGGCTGCCGCAACGCTGTGCGACCTTTGCCAGC |
| | AAGATCCAGGGCCAGGTGGACAAGATCAAGGGGGCCGGTGGTGACTAAAAAA |
| | AAAAAAAAAGCCCGGATAGCTCAGTCGGTAGAGCAGCGGCCTATGCGGCCGC |
| | CGACCAGAATCATGCAAGTGCGTAAGATAGTCGCGGGTCGGCGGCCGCATTC |
| | GAGGCCGCGTCCAGGGTTCAAGTCCCTGTTCGGGCGCCACTGCAGAAAAAAA |
| | AAAAAAAAAAACAAAAAACAAAACGGCTATTATGCGTTACCGGCGAGACGCT |
| | ACGGACTTAAATAATTGAGCCTTAAAGAAGAAATTCTTTAAGTGGATGCTCT |
| | CAAACTCAGGGAAACCTAAATCTAGTTATAGACAAGGCAATCCTGAGCCAAG |
| | CCGAAGTAGTAATTAGTAAGACCAGTGGACAATCGACG |
| SEQ ID NO. | TAATACGACTCACTATAGGGGATCCAGAGCGGCCGCTTTTTCAGCAAGATTA |
| 22: | AGCCCAGGGCAGAGCCATCTATTGCTTACATTTGCTTCTGACACAACTGTGT |
| pML44_CVB | TCACTAGCAACCTCAAACAGACACCGGGAGACCCTCGACCGTCGATTGTCCA |
| 3-GLuc- | CTGGTCAACAATAGATGACTTACAACTAATCGGAAGGTGCAGAGACTCGACG |
| | GGAGCTACCCTAACGTCAAGACGAGGGTAAAGAGAGAGTCCAATTCTCAAAG |
| | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAACAAAACACATT |
| | AAAACAGCCTGTGGGTTGATCCCACCCACAGGCCCATTGGGCGCTAGCACTC |
| | TGGTATCACGGTACCTTTGTGCGCCTGTTTTATACCCCCTCCCCCAACTGTA |
| | ACTTAGAAGTAACACACACCGATCAACAGTCAGCGTGGCACACCAGCCACGT |
| | TTTGATCAAGCACTTCTGTTACCCCGGACTGAGTATCAATAGACTGCTCACG |
| | CGGTTGAAGGAGAAAGCGTTCGTTATCCGGCCAACTACTTCGAAAAACCTAG |
| | TAACACCGTGGAAGTTGCAGAGTGTTTCGCTCAGCACTACCCCAGTGTAGAT |
| | CAGGTCGATGAGTCACCGCATTCCCCACGGGCGACCGTGGCGGTGGCTGCGT |
| | TGGCGGCCTGCCCATGGGGAAACCCATGGGACGCTCTAATACAGACATGGTG |
| | CGAAGAGTCTATTGAGCTAGTTGGTAGTCCTCCGGCCCCTGAATGCGGCTAA |
| | TCCTAACTGCGGAGCACACACCCTCAAGCCAGAGGGCAGTGTGTCGTAACGG |
| | GCAACTCTGCAGCGGAACCGACTACTTTGGGTGTCCGTGTTTCATTTTATTC |
| | CTATACTGGCTGCTTATGGTGACAATTGAGAGATCGTTACCATATAGCTATT |
| | GGATTGGCCATCCGGTGACTAATAGAGCTATTATATATCCCTTTGTTGGGTT |
| | TATACCACTTAGCTTGAAAGAGGTTAAAACATTACAATTCATTGTTAAGTTG |
| | AATACAGCAAAATGGGAGTCAAAGTTCTGTTTGCCCTGATCTGCATCGCTGT |
| | GGCCGAGGCCAAGCCCACCGAGAACAACGAAGACTTCAACATCGTGGCCGTG |
| | GCCAGCAACTTCGCGACCACGGATCTCGATGCTGACCGCGGGAAGTTGCCCG |
| | GCAAGAAGCTGCCGCTGGAGGTGCTCAAAGAGATGGAAGCCAATGCCCGGAA |
| | AGCTGGCTGCACCAGGGGCTGTCTGATCTGCCTGTCCCACATCAAGTGCACG |
| | CCCAAGATGAAGAAGTTCATCCCAGGACGCTGCCACACCTACGAAGGCGACA |
| | AAGAGTCCGCACAGGGCGGCATAGGCGAGGCGATCGTCGACATTCCTGAGAT |
| | TCCTGGGTTCAAGGACTTGGAGCCCATGGAGCAGTTCATCGCACAGGTCGAT |
| | CTGTGTGTGGACTGCACAACTGGCTGCCTCAAAGGGCTTGCCAACGTGCAGT |
| | GTTCTGACCTGCTCAAGAAGTGGCTGCCGCAACGCTGTGCGACCTTTGCCAG |
| | CAAGATCCAGGGCCAGGTGGACAAGATCAAGGGGGCCGGTGGTGACTAAAGC |
| | TCGCTTTCTTGCTGTCCAATTTCTATTAAAGGTTCCTTTGTTCCCTAAGTCC |
| | AACTACTAAACTGGGGGATATTATGAAGGGCCTTGAGCATCTGGATTCTGCC |
| | TAATAAAAAACATTTATTTTCATTGCAGCTCGCTTTCTTGCTGTCCAATTTC |
| | TATTAAAGGTTCCTTTGTTCCCTAAGTCCAACTACTAAACTGGGGGATATTA |
| | TGAAGGGCCTTGAGCATCTGGATTCTGCCTAATAAAAAACATTTATTTTCAT |
| | TGCAAAAAAAAAAAAAGCCCGGATAGCTCAGTCGGTAGAGCAGCGGCCTATG |
| | CGGCCGCCGACCAGAATCATGCAAGTGCGTAAGATAGTCGCGGGTCGGCGGC |
| | CGCATTCGAGGCCGCGTCCAGGGTTCAAGTCCCTGTTCGGGCGCCACTGCAG |
| | AAAAAAAAAAAAAAAAAACAAAAAACAAAACGGCTATTATGCGTTACCGGCG |
| | AGACGCTACGGACTTAAATAATTGAGCCTTAAAGAAGAAATTCTTTAAGTGG |
| | ATGCTCTCAAACTCAGGGAAACCTAAATCTAGTTATAGACAAGGCAATCCTG |
| | AGCCAAGCCGAAGTAGTAATTAGTAAGACCAGTGGACAATCGACG |
| SEQ ID NO. | TAATACGACTCACTATAGGGGATCCGGGAGACCCTCGACCGTCGATTGTCCA |
| 23: | CTGGTCAACAATAGATGACTTACAACTAATCGGAAGGTGCAGAGACTCGACG |
| pML45_tS1m | GGAGCTACCCTAACGTCAAGACGAGGGTAAAGAGAGAGTCCAATTCTCAAAG |
| -CVB3- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAAAAAAAAAAGCCCGGATAGCTCAGTCGGTAGAGCAGCGGCCTATGCGG |
| | CCGCCGACCAGAATCATGCAAGTGCGTAAGATAGTCGCGGGTCGGCGGCCGC |
| | ATTCGAGGCCGCGTCCAGGGTTCAAGTCCCTGTTCGGGCGCCACTGCAGAAA |
| | AAAAAAAAAAAAAACAAAAAACAAAAAAAACAAAAAAAAAACCAAAAAAACA |
| | AAACACATTAAAACAGCCTGTGGGTTGATCCCACCCACAGGCCCATTGGGCG |
| | CTAGCACTCTGGTATCACGGTACCTTTGTGCGCCTGTTTTATACCCCCTCCC |
| | CCAACTGTAACTTAGAAGTAACACACACCGATCAACAGTCAGCGTGGCACAC |
| | CAGCCACGTTTTGATCAAGCACTTCTGTTACCCCGGACTGAGTATCAATAGA |
| | CTGCTCACGCGGTTGAAGGAGAAAGCGTTCGTTATCCGGCCAACTACTTCGA |
| | AAAACCTAGTAACACCGTGGAAGTTGCAGAGTGTTTCGCTCAGCACTACCCC |
| | AGTGTAGATCAGGTCGATGAGTCACCGCATTCCCCACGGGCGACCGTGGCGG |
| | TGGCTGCGTTGGCGGCCTGCCCATGGGGAAACCCATGGGACGCTCTAATACA |
| | GACATGGTGCGAAGAGTCTATTGAGCTAGTTGGTAGTCCTCCGGCCCCTGAA |
| | TGCGGCTAATCCTAACTGCGGAGCACACACCCTCAAGCCAGAGGGCAGTGTG |
| | TCGTAACGGGCAACTCTGCAGCGGAACCGACTACTTTGGGTGTCCGTGTTTC |
| | ATTTTATTCCTATACTGGCTGCTTATGGTGACAATTGAGAGATCGTTACCAT |
| | ATAGCTATTGGATTGGCCATCCGGTGACTAATAGAGCTATTATATATCCCTT |
| | TGTTGGGTTTATACCACTTAGCTTGAAAGAGGTTAAAACATTACAATTCATT |
| | GTTAAGTTGAATACAGCAAAATGGTGAGCAAGGGCGAGGAGCTGTTCACCGG |
| | GGTGGTGCCCATCCTGGTCGAGCTGGACGGCGACGTAAACGGCCACAAGTTC |
| | AGCGTGTCTGGCGAGGGCGAGGGCGATGCCACCTACGGCAAGCTGACCCTGA |
| | AGTTCATCTGCACCACCGGCAAGCTGCCCGTGCCCTGGCCCACCCTCGTGAC |
| | CACCCTGACCTACGGCGTGCAGTGCTTCAGCCGCTACCCCGACCACATGAAG |
| | CAGCACGACTTCTTCAAGTCCGCCATGCCCGAAGGCTACGTCCAGGAGCGCA |
| | CCATCTTCTTCAAGGACGACGGCAACTACAAGACCCGCGCCGAGGTGAAGTT |
| | CGAGGGCGACACCCTGGTGAACCGCATCGAGCTGAAGGGCATCGACTTCAAG |
| | GAGGACGGCAACATCCTGGGGCACAAGCTGGAGTACAACTACAACAGCCACA |
| | ACGTCTATATCATGGCCGACAAGCAGAAGAACGGCATCAAGGCGAACTTCAA |
| | GATCCGCCACAACATCGAGGACGGCAGCGTGCAGCTCGCCGACCACTACCAG |
| | CAGAACACCCCCATCGGCGACGGCCCCGTGCTGCTGCCCGACAACCACTACC |
| | TGAGCACCCAGTCCGCCCTGAGCAAAGACCCCAACGAGAAGCGCGATCACAT |
| | GGTCCTGCTGGAGTTCGTGACCGCCGCCGGGATCACTCTCGGCATGGACGAG |
| | CTGTACAAGTAAAAAAAACAAAAAACAAAACGGCTATTATGCGTTACCGGCG |
| | AGACGCTACGGACTTAAATAATTGAGCCTTAAAGAAGAAATTCTTTAAGTGG |
| | ATGCTCTCAAACTCAGGGAAACCTAAATCTAGTTATAGACAAGGCAATCCTG |
| | AGCCAAGCCGAAGTAGTAATTAGTAAGACCAGTGGACAATCGACG |
| SEQ ID NO. | TAATACGACTCACTATAGGGGATCCGGGAGACCCTCGACCGTCGATTGTCCA |
| 24: | CTGGTCAACAATAGATGACTTACAACTAATCGGAAGGTGCAGAGACTCGACG |
| pML46_CVB | GGAGCTACCCTAACGTCAAGACGAGGGTAAAGAGAGAGTCCAATTCTCAAAG |
| 3-GLuc- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAACAAAACACATT |
| | AAAACAGCCTGTGGGTTGATCCCACCCACAGGCCCATTGGGCGCTAGCACTC |
| | TGGTATCACGGTACCTTTGTGCGCCTGTTTTATACCCCCTCCCCCAACTGTA |
| | ACTTAGAAGTAACACACACCGATCAACAGTCAGCGTGGCACACCAGCCACGT |
| | TTTGATCAAGCACTTCTGTTACCCCGGACTGAGTATCAATAGACTGCTCACG |
| | CGGTTGAAGGAGAAAGCGTTCGTTATCCGGCCAACTACTTCGAAAAACCTAG |
| | TAACACCGTGGAAGTTGCAGAGTGTTTCGCTCAGCACTACCCCAGTGTAGAT |
| | CAGGTCGATGAGTCACCGCATTCCCCACGGGCGACCGTGGCGGTGGCTGCGT |
| | TGGCGGCCTGCCCATGGGGAAACCCATGGGACGCTCTAATACAGACATGGTG |
| | CGAAGAGTCTATTGAGCTAGTTGGTAGTCCTCCGGCCCCTGAATGCGGCTAA |
| | TCCTAACTGCGGAGCACACACCCTCAAGCCAGAGGGCAGTGTGTCGTAACGG |
| | GCAACTCTGCAGCGGAACCGACTACTTTGGGTGTCCGTGTTTCATTTTATTC |
| | CTATACTGGCTGCTTATGGTGACAATTGAGAGATCGTTACCATATAGCTATT |
| | GGATTGGCCATCCGGTGACTAATAGAGCTATTATATATCCCTTTGTTGGGTT |
| | TATACCACTTAGCTTGAAAGAGGTTAAAACATTACAATTCATTGTTAAGTTG |
| | AATACAGCAAAATGGGAGTCAAAGTTCTGTTTGCCCTGATCTGCATCGCTGT |
| | GGCCGAGGCCAAGCCCACCGAGAACAACGAAGACTTCAACATCGTGGCCGTG |
| | GCCAGCAACTTCGCGACCACGGATCTCGATGCTGACCGCGGGAAGTTGCCCG |
| | GCAAGAAGCTGCCGCTGGAGGTGCTCAAAGAGATGGAAGCCAATGCCCGGAA |
| | AGCTGGCTGCACCAGGGGCTGTCTGATCTGCCTGTCCCACATCAAGTGCACG |
| | CCCAAGATGAAGAAGTTCATCCCAGGACGCTGCCACACCTACGAAGGCGACA |
| | AAGAGTCCGCACAGGGGGCATAGGCGAGGCGATCGTCGACATTCCTGAGATT |
| | CCTGGGTTCAAGGACTTGGAGCCCATGGAGCAGTTCATCGCACAGGTCGATC |
| | TGTGTGTGGACTGCACAACTGGCTGCCTCAAAGGGCTTGCCAACGTGCAGTG |
| | TTCTGACCTGCTCAAGAAGTGGCTGCCGCAACGCTGTGCGACCTTTGCCAGC |
| | AAGATCCAGGGCCAGGTGGACAAGATCAAGGGGGCCGGTGGTGACTAAAAAA |
| | AACAAAAAACAAAACGGCTATTATGCGTTACCGGCGAGACGCTACGGACTTA |
| | AATAATTGAGCCTTAAAGAAGAAATTCTTTAAGTGGATGCTCTCAAACTCAG |
| | GGAAACCTAAATCTAGTTATAGACAAGGCAATCCTGAGCCAAGCCGAAGTAG |
| | TAATTAGTAAGACCAGTGGACAATCGACG |
| SEQ ID NO. | TAATACGACTCACTATAGGGGATCCGGGAGACCCTCGACCGTCGATTGTCCA |
| 25: | CTGGTCAACAATAGATGACTTACAACTAATCGGAAGGTGCAGAGACTCGACG |
| pML47_CVB | GGAGCTACCCTAACGTCAAGACGAGGGTAAAGAGAGAGTCCAATTCTCAAAG |
| 3-EGFP- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAAAAAAAAAAAACCAAAAAAACAAAACACATTA |
| | AAACAGCCTGTGGGTTGATCCCACCCACAGGCCCATTGGGCGCTAGCACTCT |
| | GGTATCACGGTACCTTTGTGCGCCTGTTTTATACCCCCTCCCCCAACTGTAA |
| | CTTAGAAGTAACACACACCGATCAACAGTCAGCGTGGCACACCAGCCACGTT |
| | TTGATCAAGCACTTCTGTTACCCCGGACTGAGTATCAATAGACTGCTCACGC |
| | GGTTGAAGGAGAAAGCGTTCGTTATCCGGCCAACTACTTCGAAAAACCTAGT |
| | AACACCGTGGAAGTTGCAGAGTGTTTCGCTCAGCACTACCCCAGTGTAGATC |
| | AGGTCGATGAGTCACCGCATTCCCCACGGGCGACCGTGGCGGTGGCTGCGTT |
| | GGCGGCCTGCCCATGGGGAAACCCATGGGACGCTCTAATACAGACATGGTGC |
| | GAAGAGTCTATTGAGCTAGTTGGTAGTCCTCCGGCCCCTGAATGCGGCTAAT |
| | CCTAACTGCGGAGCACACACCCTCAAGCCAGAGGGCAGTGTGTCGTAACGGG |
| | CAACTCTGCAGCGGAACCGACTACTTTGGGTGTCCGTGTTTCATTTTATTCC |
| | TATACTGGCTGCTTATGGTGACAATTGAGAGATCGTTACCATATAGCTATTG |
| | GATTGGCCATCCGGTGACTAATAGAGCTATTATATATCCCTTTGTTGGGTTT |
| | ATACCACTTAGCTTGAAAGAGGTTAAAACATTACAATTCATTGTTAAGTTGA |
| | ATACAGCAAAATGGTGAGCAAGGGCGAGGAGCTGTTCACCGGGGTGGTGCCC |
| | ATCCTGGTCGAGCTGGACGGCGACGTAAACGGCCACAAGTTCAGCGTGTCTG |
| | GCGAGGGCGAGGGCGATGCCACCTACGGCAAGCTGACCCTGAAGTTCATCTG |
| | CACCACCGGCAAGCTGCCCGTGCCCTGGCCCACCCTCGTGACCACCCTGACC |
| | TACGGCGTGCAGTGCTTCAGCCGCTACCCCGACCACATGAAGCAGCACGACT |
| | TCTTCAAGTCCGCCATGCCCGAAGGCTACGTCCAGGAGCGCACCATCTTCTT |
| | CAAGGACGACGGCAACTACAAGACCCGCGCCGAGGTGAAGTTCGAGGGCGAC |
| | ACCCTGGTGAACCGCATCGAGCTGAAGGGCATCGACTTCAAGGAGGACGGCA |
| | ACATCCTGGGGCACAAGCTGGAGTACAACTACAACAGCCACAACGTCTATAT |
| | CATGGCCGACAAGCAGAAGAACGGCATCAAGGCGAACTTCAAGATCCGCCAC |
| | AACATCGAGGACGGCAGCGTGCAGCTCGCCGACCACTACCAGCAGAACACCC |
| | CCATCGGCGACGGCCCCGTGCTGCTGCCCGACAACCACTACCTGAGCACCCA |
| | GTCCGCCCTGAGCAAAGACCCCAACGAGAAGCGCGATCACATGGTCCTGCTG |
| | GAGTTCGTGACCGCCGCCGGGATCACTCTCGGCATGGACGAGCTGTACAAGT |
| | AAAAAAAACAAAAAACAAAACGGCTATTATGCGTTACCGGCGAGACGCTACG |
| | GACTTAAATAATTGAGCCTTAAAGAAGAAATTCTTTAAGTGGATGCTCTCAA |
| | ACTCAGGGAAACCTAAATCTAGTTATAGACAAGGCAATCCTGAGCCAAGCCG |
| | AAGTAGTAATTAGTAAGACCAGTGGACAATCGACG |
| SEQ ID NO. | TAATACGACTCACTATAGGGGATCCGGGAGACCCTCGACCGTCGATTGTCCA |
| 26: | CTGGTCAACAATAGATGACTTACAACTAATCGGAAGGTGCAGAGACTCGACG |
| pML48_CVB | GGAGCTACCCTAACGTCAAGACGAGGGTAAAGAGAGAGTCCAATTCTCAAAG |
| 3-EGFP- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAAAAAAAAAAAACCAAAAAAACAAAACACATTA |
| | AAACAGCCTGTGGGTTGATCCCACCCACAGGCCCATTGGGCGCTAGCACTCT |
| | GGTATCACGGTACCTTTGTGCGCCTGTTTTATACCCCCTCCCCCAACTGTAA |
| | CTTAGAAGTAACACACACCGATCAACAGTCAGCGTGGCACACCAGCCACGTT |
| | TTGATCAAGCACTTCTGTTACCCCGGACTGAGTATCAATAGACTGCTCACGC |
| | GGTTGAAGGAGAAAGCGTTCGTTATCCGGCCAACTACTTCGAAAAACCTAGT |
| | AACACCGTGGAAGTTGCAGAGTGTTTCGCTCAGCACTACCCCAGTGTAGATC |
| | AGGTCGATGAGTCACCGCATTCCCCACGGGCGACCGTGGCGGTGGCTGCGTT |
| | GGCGGCCTGCCCATGGGGAAACCCATGGGACGCTCTAATACAGACATGGTGC |
| | GAAGAGTCTATTGAGCTAGTTGGTAGTCCTCCGGCCCCTGAATGCGGCTAAT |
| | CCTAACTGCGGAGCACACACCCTCAAGCCAGAGGGCAGTGTGTCGTAACGGG |
| | CAACTCTGCAGCGGAACCGACTACTTTGGGTGTCCGTGTTTCATTTTATTCC |
| | TATACTGGCTGCTTATGGTGACAATTGAGAGATCGTTACCATATAGCTATTG |
| | GATTGGCCATCCGGTGACTAATAGAGCTATTATATATCCCTTTGTTGGGTTT |
| | ATACCACTTAGCTTGAAAGAGGTTAAAACATTACAATTCATTGTTAAGTTGA |
| | ATACAGCAAAATGGTGAGCAAGGGCGAGGAGCTGTTCACCGGGGTGGTGCCC |
| | ATCCTGGTCGAGCTGGACGGCGACGTAAACGGCCACAAGTTCAGCGTGTCTG |
| | GCGAGGGCGAGGGCGATGCCACCTACGGCAAGCTGACCCTGAAGTTCATCTG |
| | CACCACCGGCAAGCTGCCCGTGCCCTGGCCCACCCTCGTGACCACCCTGACC |
| | TACGGCGTGCAGTGCTTCAGCCGCTACCCCGACCACATGAAGCAGCACGACT |
| | TCTTCAAGTCCGCCATGCCCGAAGGCTACGTCCAGGAGCGCACCATCTTCTT |
| | CAAGGACGACGGCAACTACAAGACCCGCGCCGAGGTGAAGTTCGAGGGCGAC |
| | ACCCTGGTGAACCGCATCGAGCTGAAGGGCATCGACTTCAAGGAGGACGGCA |
| | ACATCCTGGGGCACAAGCTGGAGTACAACTACAACAGCCACAACGTCTATAT |
| | CATGGCCGACAAGCAGAAGAACGGCATCAAGGCGAACTTCAAGATCCGCCAC |
| | AACATCGAGGACGGCAGCGTGCAGCTCGCCGACCACTACCAGCAGAACACCC |
| | CCATCGGCGACGGCCCCGTGCTGCTGCCCGACAACCACTACCTGAGCACCCA |
| | GTCCGCCCTGAGCAAAGACCCCAACGAGAAGCGCGATCACATGGTCCTGCTG |
| | GAGTTCGTGACCGCCGCCGGGATCACTCTCGGCATGGACGAGCTGTACAAGT |
| | AAAAAAAACAAAAAACAAAACGGCTATTATGCGTTACCGGCGAGACGCTACG |
| | GACTTAAATAATTGAGCCTTAAAGAAGAAATTCTTTAAGTGGATGCTCTCAA |
| | ACTCAGGGAAACCTAAATCTAGTTATAGACAAGGCAATCCTGAGCCAAGCCG |
| | AAGTAGTAATTAGTAAGACCAGTGGACAATCGACGGATAACAGCATATCTAG |
| | TAAGTAGAAAATGCGGCCGCCGACCAGAATCATGCAAGTGCGTAAGATAGTC |
| | GCGGGTCGGCGGCCGCATCTGCTGGGAAGCTACGATCCGTAGAAAATGCGGC |
| | CGCCGACCAGAATCATGCAAGTGCGTAAGATAGTCGCGGGTCGGCGGCCGCA |
| | TCTGCTGGGTAGCTGTGAACCGTAGAAAATGCGGCCGCCGACCAGAATCATG |
| | CAAGTGCGTAAGATAGTCGCGGGTCGGCGGCCGCATCTGCTGGGAAGCTACG |
| | ATCCGTAGAAAATGCGGCCGCCGACCAGAATCATGCAAGTGCGTAAGATAGT |
| | CGCGGGTCGGCGGCCGCAT |
| SEQ ID NO. | TAATACGACTCACTATAGGGGATCCGGGAGACCCTCGACCGTCGATTGTCCA |
| 27: | CTGGTCAACAATAGATGACTTACAACTAATCGGAAGGTGCAGAGACTCGACG |
| pML49_CVB | GGAGCTACCCTAACGTCAAGACGAGGGTAAAGAGAGAGTCCAATTCTCAAAG |
| 3-GLuc- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAACAAAACACATT |
| | AAAACAGCCTGTGGGTTGATCCCACCCACAGGCCCATTGGGCGCTAGCACTC |
| | TGGTATCACGGTACCTTTGTGCGCCTGTTTTATACCCCCTCCCCCAACTGTA |
| | ACTTAGAAGTAACACACACCGATCAACAGTCAGCGTGGCACACCAGCCACGT |
| | TTTGATCAAGCACTTCTGTTACCCCGGACTGAGTATCAATAGACTGCTCACG |
| | CGGTTGAAGGAGAAAGCGTTCGTTATCCGGCCAACTACTTCGAAAAACCTAG |
| | TAACACCGTGGAAGTTGCAGAGTGTTTCGCTCAGCACTACCCCAGTGTAGAT |
| | CAGGTCGATGAGTCACCGCATTCCCCACGGGCGACCGTGGCGGTGGCTGCGT |
| | TGGCGGCCTGCCCATGGGGAAACCCATGGGACGCTCTAATACAGACATGGTG |
| | CGAAGAGTCTATTGAGCTAGTTGGTAGTCCTCCGGCCCCTGAATGCGGCTAA |
| | TCCTAACTGCGGAGCACACACCCTCAAGCCAGAGGGCAGTGTGTCGTAACGG |
| | GCAACTCTGCAGCGGAACCGACTACTTTGGGTGTCCGTGTTTCATTTTATTC |
| | CTATACTGGCTGCTTATGGTGACAATTGAGAGATCGTTACCATATAGCTATT |
| | GGATTGGCCATCCGGTGACTAATAGAGCTATTATATATCCCTTTGTTGGGTT |
| | TATACCACTTAGCTTGAAAGAGGTTAAAACATTACAATTCATTGTTAAGTTG |
| | AATACAGCAAAATGGGAGTCAAAGTTCTGTTTGCCCTGATCTGCATCGCTGT |
| | GGCCGAGGCCAAGCCCACCGAGAACAACGAAGACTTCAACATCGTGGCCGTG |
| | GCCAGCAACTTCGCGACCACGGATCTCGATGCTGACCGCGGGAAGTTGCCCG |
| | GCAAGAAGCTGCCGCTGGAGGTGCTCAAAGAGATGGAAGCCAATGCCCGGAA |
| | AGCTGGCTGCACCAGGGGCTGTCTGATCTGCCTGTCCCACATCAAGTGCACG |
| | CCCAAGATGAAGAAGTTCATCCCAGGACGCTGCCACACCTACGAAGGCGACA |
| | AAGAGTCCGCACAGGGGGGCATAGGCGAGGCGATCGTCGACATTCCTGAGAT |
| | TCCTGGGTTCAAGGACTTGGAGCCCATGGAGCAGTTCATCGCACAGGTCGAT |
| | CTGTGTGTGGACTGCACAACTGGCTGCCTCAAAGGGCTTGCCAACGTGCAGT |
| | GTTCTGACCTGCTCAAGAAGTGGCTGCCGCAACGCTGTGCGACCTTTGCCAG |
| | CAAGATCCAGGGCCAGGTGGACAAGATCAAGGGGGCCGGTGGTGACTAAAAA |
| | AAACAAAAAACAAAACGGCTATTATGCGTTACCGGCGAGACGCTACGGACTT |
| | AAATAATTGAGCCTTAAAGAAGAAATTCTTTAAGTGGATGCTCTCAAACTCA |
| | GGGAAACCTAAATCTAGTTATAGACAAGGCAATCCTGAGCCAAGCCGAAGTA |
| | GTAATTAGTAAGACCAGTGGACAATCGACGGATAACAGCATATCTAGTAAGT |
| | AGAAAATGCGGCCGCCGACCAGAATCATGCAAGTGCGTAAGATAGTCGCGGG |
| | TCGGCGGCCGCATCTGCTGGGAAGCTACGATCCGTAGAAAATGCGGCCGCCG |
| | ACCAGAATCATGCAAGTGCGTAAGATAGTCGCGGGTCGGCGGCCGCATCTGC |
| | TGGGTAGCTGTGAACCGTAGAAAATGCGGCCGCCGACCAGAATCATGCAAGT |
| | GCGTAAGATAGTCGCGGGTCGGCGGCCGCATCTGCTGGGAAGCTACGATCCG |
| | TAGAAAATGCGGCCGCCGACCAGAATCATGCAAGTGCGTAAGATAGTCGCGG |
| | GTCGGCGGCCGCAT |
| SEQ ID NO. | TAATACGACTCACTATAGGGGATCCGGGAGACCCTCGACCGTCGATTGTCCA |
| 28: | CTGGTCAACAATAGATGACTTACAACTAATCGGAAGGTGCAGAGACTCGACG |
| pML50_CVB | GGAGCTACCCTAACGTCAAGACGAGGGTAAAGAGAGAGTCCAATTCTCAAAG |
| 3-EGFP- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAACAAAACACATT |
| | AAAACAGCCTGTGGGTTGATCCCACCCACAGGCCCATTGGGCGCTAGCACTC |
| | TGGTATCACGGTACCTTTGTGCGCCTGTTTTATACCCCCTCCCCCAACTGTA |
| | ACTTAGAAGTAACACACACCGATCAACAGTCAGCGTGGCACACCAGCCACGT |
| | TTTGATCAAGCACTTCTGTTACCCCGGACTGAGTATCAATAGACTGCTCACG |
| | CGGTTGAAGGAGAAAGCGTTCGTTATCCGGCCAACTACTTCGAAAAACCTAG |
| | TAACACCGTGGAAGTTGCAGAGTGTTTCGCTCAGCACTACCCCAGTGTAGAT |
| | CAGGTCGATGAGTCACCGCATTCCCCACGGGCGACCGTGGCGGTGGCTGCGT |
| | TGGCGGCCTGCCCATGGGGAAACCCATGGGACGCTCTAATACAGACATGGTG |
| | CGAAGAGTCTATTGAGCTAGTTGGTAGTCCTCCGGCCCCTGAATGCGGCTAA |
| | TCCTAACTGCGGAGCACACACCCTCAAGCCAGAGGGCAGTGTGTCGTAACGG |
| | GCAACTCTGCAGCGGAACCGACTACTTTGGGTGTCCGTGTTTCATTTTATTC |
| | CTATACTGGCTGCTTATGGTGACAATTGAGAGATCGTTACCATATAGCTATT |
| | GGATTGGCCATCCGGTGACTAATAGAGCTATTATATATCCCTTTGTTGGGTT |
| | TATACCACTTAGCTTGAAAGAGGTTAAAACATTACAATTCATTGTTAAGTTG |
| | AATACAGCAAAATGGTGAGCAAGGGCGAGGAGCTGTTCACCGGGGTGGTGCC |
| | CATCCTGGTCGAGCTGGACGGCGACGTAAACGGCCACAAGTTCAGCGTGTCT |
| | GGCGAGGGCGAGGGCGATGCCACCTACGGCAAGCTGACCCTGAAGTTCATCT |
| | GCACCACCGGCAAGCTGCCCGTGCCCTGGCCCACCCTCGTGACCACCCTGAC |
| | CTACGGCGTGCAGTGCTTCAGCCGCTACCCCGACCACATGAAGCAGCACGAC |
| | TTCTTCAAGTCCGCCATGCCCGAAGGCTACGTCCAGGAGCGCACCATCTTCT |
| | TCAAGGACGACGGCAACTACAAGACCCGCGCCGAGGTGAAGTTCGAGGGCGA |
| | CACCCTGGTGAACCGCATCGAGCTGAAGGGCATCGACTTCAAGGAGGACGGC |
| | AACATCCTGGGGCACAAGCTGGAGTACAACTACAACAGCCACAACGTCTATA |
| | TCATGGCCGACAAGCAGAAGAACGGCATCAAGGCGAACTTCAAGATCCGCCA |
| | CAACATCGAGGACGGCAGCGTGCAGCTCGCCGACCACTACCAGCAGAACACC |
| | CCCATCGGCGACGGCCCCGTGCTGCTGCCCGACAACCACTACCTGAGCACCC |
| | AGTCCGCCCTGAGCAAAGACCCCAACGAGAAGCGCGATCACATGGTCCTGCT |
| | GGAGTTCGTGACCGCCGCCGGGATCACTCTCGGCATGGACGAGCTGTACAAG |
| | TAAAAAAAACAAAAAACAAAACGGCTATTATGCGTTACCGGCGAGACGCTAC |
| | GGACTTAAATAATTGAGCCTTAAAGAAGAAATTCTTTAAGTGGATGCTCTCA |
| | AACTCAGGGAAACCTAAATCTAGTTATAGACAAGGCAATCCTGAGCCAAGCC |
| | GAAGTAGTAATTAGTAAGACCAGTGGACAATCGACGGATAACAGCATATCTA |
| | AAAAAAAAAAAAGCCCGGATAGCTCAGTCGGTAGAGCAGCGGCCTATGCGGC |
| | CGCCGACCAGAATCATGCAAGTGCGTAAGATAGTCGCGGGTCGGCGGCCGCA |
| | TTCGAGGCCGCGTCCAGGGTTCAAGTCCCTGTTCGGGCGCCA |
| SEQ ID NO. | TAATACGACTCACTATAGGGGATCCGGGAGACCCTCGACCGTCGATTGTCCA |
| 29: | CTGGTCAACAATAGATGACTTACAACTAATCGGAAGGTGCAGAGACTCGACG |
| pML51_CVB | GGAGCTACCCTAACGTCAAGACGAGGGTAAAGAGAGAGTCCAATTCTCAAAG |
| 3-GLuc- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAAAACAAAAAACAAAAAAAACAAAAAAAAAACCAAAAAAACAAAACACATT |
| | AAAACAGCCTGTGGGTTGATCCCACCCACAGGCCCATTGGGCGCTAGCACTC |
| | TGGTATCACGGTACCTTTGTGCGCCTGTTTTATACCCCCTCCCCCAACTGTA |
| | ACTTAGAAGTAACACACACCGATCAACAGTCAGCGTGGCACACCAGCCACGT |
| | TTTGATCAAGCACTTCTGTTACCCCGGACTGAGTATCAATAGACTGCTCACG |
| | CGGTTGAAGGAGAAAGCGTTCGTTATCCGGCCAACTACTTCGAAAAACCTAG |
| | TAACACCGTGGAAGTTGCAGAGTGTTTCGCTCAGCACTACCCCAGTGTAGAT |
| | CAGGTCGATGAGTCACCGCATTCCCCACGGGCGACCGTGGCGGTGGCTGCGT |
| | TGGCGGCCTGCCCATGGGGAAACCCATGGGACGCTCTAATACAGACATGGTG |
| | CGAAGAGTCTATTGAGCTAGTTGGTAGTCCTCCGGCCCCTGAATGCGGCTAA |
| | TCCTAACTGCGGAGCACACACCCTCAAGCCAGAGGGCAGTGTGTCGTAACGG |
| | GCAACTCTGCAGCGGAACCGACTACTTTGGGTGTCCGTGTTTCATTTTATTC |
| | CTATACTGGCTGCTTATGGTGACAATTGAGAGATCGTTACCATATAGCTATT |
| | GGATTGGCCATCCGGTGACTAATAGAGCTATTATATATCCCTTTGTTGGGTT |
| | TATACCACTTAGCTTGAAAGAGGTTAAAACATTACAATTCATTGTTAAGTTG |
| | AATACAGCAAAATGGGAGTCAAAGTTCTGTTTGCCCTGATCTGCATCGCTGT |
| | GGCCGAGGCCAAGCCCACCGAGAACAACGAAGACTTCAACATCGTGGCCGTG |
| | GCCAGCAACTTCGCGACCACGGATCTCGATGCTGACCGCGGGAAGTTGCCCG |
| | GCAAGAAGCTGCCGCTGGAGGTGCTCAAAGAGATGGAAGCCAATGCCCGGAA |
| | AGCTGGCTGCACCAGGGGCTGTCTGATCTGCCTGTCCCACATCAAGTGCACG |
| | CCCAAGATGAAGAAGTTCATCCCAGGACGCTGCCACACCTACGAAGGCGACA |
| | AAGAGTCCGCACAGGGGGGCATAGGCGAGGCGATCGTCGACATTCCTGAGAT |
| | TCCTGGGTTCAAGGACTTGGAGCCCATGGAGCAGTTCATCGCACAGGTCGAT |
| | CTGTGTGTGGACTGCACAACTGGCTGCCTCAAAGGGCTTGCCAACGTGCAGT |
| | GTTCTGACCTGCTCAAGAAGTGGCTGCCGCAACGCTGTGCGACCTTTGCCAG |
| | CAAGATCCAGGGCCAGGTGGACAAGATCAAGGGGGCCGGTGGTGACTAAAAA |
| | AAACAAAAAACAAAACGGCTATTATGCGTTACCGGCGAGACGCTACGGACTT |
| | AAATAATTGAGCCTTAAAGAAGAAATTCTTTAAGTGGATGCTCTCAAACTCA |
| | GGGAAACCTAAATCTAGTTATAGACAAGGCAATCCTGAGCCAAGCCGAAGTA |
| | GTAATTAGTAAGACCAGTGGACAATCGACGGATAACAGCATATCTAAAAAAA |
| | AAAAAAGCCCGGATAGCTCAGTCGGTAGAGCAGCGGCCTATGCGGCCGCCGA |
| | CCAGAATCATGCAAGTGCGTAAGATAGTCGCGGGTCGGCGGCCGCATTCGAG |
| | GCCGCGTCCAGGGTTCAAGTCCCTGTTCGGGCGCCA |
| SEQ ID NO. | TAATACGACTCACTATAGGGGATCCGGGAGACCCTCGACCGTCGATTGTCCA |
| 30: | CTGGTCAACAATAGATGACTTACAACTAATCGGAAGGTGCAGAGACTCGACG |
| pML75_EGF | GGAGCTACCCTAACGTCAAGACGAGGGTAAAGAGAGAGTCCAATTCTCAAAG |
| P-CVB3- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | AAACAAAAAACAAAAAAAACAAAAAAAAAACCAAAAAAACAAAACACAATGG |
| | TGAGCAAGGGCGAGGAGCTGTTCACCGGGGTGGTGCCCATCCTGGTCGAGCT |
| | GGACGGCGACGTAAACGGCCACAAGTTCAGCGTGTCTGGCGAGGGCGAGGGC |
| | GATGCCACCTACGGCAAGCTGACCCTGAAGTTCATCTGCACCACCGGCAAGC |
| | TGCCCGTGCCCTGGCCCACCCTCGTGACCACCCTGACCTACGGCGTGCAGTG |
| | CTTCAGCCGCTACCCCGACCACATGAAGCAGCACGACTTCTTCAAGTCCGCC |
| | ATGCCCGAAGGCTACGTCCAGGAGCGCACCATCTTCTTCAAGGACGACGGCA |
| | ACTACAAGACCCGCGCCGAGGTGAAGTTCGAGGGCGACACCCTGGTGAACCG |
| | CATCGAGCTGAAGGGCATCGACTTCAAGGAGGACGGCAACATCCTGGGGCAC |
| | AAGCTGGAGTACAACTACAACAGCCACAACGTCTATATCATGGCCGACAAGC |
| | AGAAGAACGGCATCAAGGCGAACTTCAAGATCCGCCACAACATCGAGGACGG |
| | CAGCGTGCAGCTCGCCGACCACTACCAGCAGAACACCCCCATCGGCGACGGC |
| | CCCGTGCTGCTGCCCGACAACCACTACCTGAGCACCCAGTCCGCCCTGAGCA |
| | AAGACCCCAACGAGAAGCGCGATCACATGGTCCTGCTGGAGTTCGTGACCGC |
| | CGCCGGGATCACTCTCGGCATGGACGAGCTGTACAAGTAATTAAAACAGCCT |
| | GTGGGTTGATCCCACCCACAGGCCCATTGGGCGCTAGCACTCTGGTATCACG |
| | GTACCTTTGTGCGCCTGTTTTATACCCCCTCCCCCAACTGTAACTTAGAAGT |
| | AACACACACCGATCAACAGTCAGCGTGGCACACCAGCCACGTTTTGATCAAG |
| | CACTTCTGTTACCCCGGACTGAGTATCAATAGACTGCTCACGCGGTTGAAGG |
| | AGAAAGCGTTCGTTATCCGGCCAACTACTTCGAAAAACCTAGTAACACCGTG |
| | GAAGTTGCAGAGTGTTTCGCTCAGCACTACCCCAGTGTAGATCAGGTCGATG |
| | AGTCACCGCATTCCCCACGGGCGACCGTGGCGGTGGCTGCGTTGGCGGCCTG |
| | CCCATGGGGAAACCCATGGGACGCTCTAATACAGACATGGTGCGAAGAGTCT |
| | ATTGAGCTAGTTGGTAGTCCTCCGGCCCCTGAATGCGGCTAATCCTAACTGC |
| | GGAGCACACACCCTCAAGCCAGAGGGCAGTGTGTCGTAACGGGCAACTCTGC |
| | AGCGGAACCGACTACTTTGGGTGTCCGTGTTTCATTTTATTCCTATACTGGC |
| | TGCTTATGGTGACAATTGAGAGATCGTTACCATATAGCTATTGGATTGGCCA |
| | TCCGGTGACTAATAGAGCTATTATATATCCCTTTGTTGGGTTTATACCACTT |
| | AGCTTGAAAGAGGTTAAAACATTACAATTCATTGTTAAGTTGAATACAGCAA |
| | AAAAAAACAAAAAACAAAACGGCTATTATGCGTTACCGGCGAGACGCTACGG |
| | ACTTAAATAATTGAGCCTTAAAGAAGAAATTCTTTAAGTGGATGCTCTCAAA |
| | CTCAGGGAAACCTAAATCTAGTTATAGACAAGGCAATCCTGAGCCAAGCCGA |
| | AGTAGTAATTAGTAAGACCAGTGGACAATCGACG |
| SEQ ID NO. | TAATACGACTCACTATAGGGGATCCGGGAGACCCTCGACCGTCGATTGTCCA |
| 31: | CTGGTCAACAATAGATGACTTACAACTAATCGGAAGGTGCAGAGACTCGACG |
| pML76_EGF | GGAGCTACCCTAACGTCAAGACGAGGGTAAAGAGAGAGTCCAATTCTCAAAG |
| P-CVB3- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | ACAAAAAAAAAAAAAAAAAACCAAAAAAACAAAACACAATGGTGAGCAAGGG |
| | CGAGGAGCTGTTCACCGGGGTGGTGCCCATCCTGGTCGAGCTGGACGGCGAC |
| | GTAAACGGCCACAAGTTCAGCGTGTCTGGCGAGGGCGAGGGCGATGCCACCT |
| | ACGGCAAGCTGACCCTGAAGTTCATCTGCACCACCGGCAAGCTGCCCGTGCC |
| | CTGGCCCACCCTCGTGACCACCCTGACCTACGGCGTGCAGTGCTTCAGCCGC |
| | TACCCCGACCACATGAAGCAGCACGACTTCTTCAAGTCCGCCATGCCCGAAG |
| | GCTACGTCCAGGAGCGCACCATCTTCTTCAAGGACGACGGCAACTACAAGAC |
| | CCGCGCCGAGGTGAAGTTCGAGGGCGACACCCTGGTGAACCGCATCGAGCTG |
| | AAGGGCATCGACTTCAAGGAGGACGGCAACATCCTGGGGCACAAGCTGGAGT |
| | ACAACTACAACAGCCACAACGTCTATATCATGGCCGACAAGCAGAAGAACGG |
| | CATCAAGGCGAACTTCAAGATCCGCCACAACATCGAGGACGGCAGCGTGCAG |
| | CTCGCCGACCACTACCAGCAGAACACCCCCATCGGCGACGGCCCCGTGCTGC |
| | TGCCCGACAACCACTACCTGAGCACCCAGTCCGCCCTGAGCAAAGACCCCAA |
| | CGAGAAGCGCGATCACATGGTCCTGCTGGAGTTCGTGACCGCCGCCGGGATC |
| | ACTCTCGGCATGGACGAGCTGTACAAGTAATTAAAACAGCCTGTGGGTTGAT |
| | CCCACCCACAGGCCCATTGGGCGCTAGCACTCTGGTATCACGGTACCTTTGT |
| | GCGCCTGTTTTATACCCCCTCCCCCAACTGTAACTTAGAAGTAACACACACC |
| | GATCAACAGTCAGCGTGGCACACCAGCCACGTTTTGATCAAGCACTTCTGTT |
| | ACCCCGGACTGAGTATCAATAGACTGCTCACGCGGTTGAAGGAGAAAGCGTT |
| | CGTTATCCGGCCAACTACTTCGAAAAACCTAGTAACACCGTGGAAGTTGCAG |
| | AGTGTTTCGCTCAGCACTACCCCAGTGTAGATCAGGTCGATGAGTCACCGCA |
| | TTCCCCACGGGCGACCGTGGCGGTGGCTGCGTTGGCGGCCTGCCCATGGGGA |
| | AACCCATGGGACGCTCTAATACAGACATGGTGCGAAGAGTCTATTGAGCTAG |
| | TTGGTAGTCCTCCGGCCCCTGAATGCGGCTAATCCTAACTGCGGAGCACACA |
| | CCCTCAAGCCAGAGGGCAGTGTGTCGTAACGGGCAACTCTGCAGCGGAACCG |
| | ACTACTTTGGGTGTCCGTGTTTCATTTTATTCCTATACTGGCTGCTTATGGT |
| | GACAATTGAGAGATCGTTACCATATAGCTATTGGATTGGCCATCCGGTGACT |
| | AATAGAGCTATTATATATCCCTTTGTTGGGTTTATACCACTTAGCTTGAAAG |
| | AGGTTAAAACATTACAATTCATTGTTAAGTTGAATACAGCAAAAAAAAACAA |
| | AAAACAAAACGGCTATTATGCGTTACCGGCGAGACGCTACGGACTTAAATAA |
| | TTGAGCCTTAAAGAAGAAATTCTTTAAGTGGATGCTCTCAAACTCAGGGAAA |
| | CCTAAATCTAGTTATAGACAAGGCAATCCTGAGCCAAGCCGAAGTAGTAATT |
| | AGTAAGACCAGTGGACAATCGACG |
| SEQ ID NO. | TAATACGACTCACTATAGGGGATCCGGGAGACCCTCGACCGTCGATTGTCCA |
| 32: | CTGGTCAACAATAGATGACTTACAACTAATCGGAAGGTGCAGAGACTCGACG |
| pML77_EGF | GGAGCTACCCTAACGTCAAGACGAGGGTAAAGAGAGAGTCCAATTCTCAAAG |
| P-CVB3- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | ACAAAAAAAAAACCAAAAAAAAAAACACAATGGTGAGCAAGGGCGAGGAGCT |
| | GTTCACCGGGGTGGTGCCCATCCTGGTCGAGCTGGACGGCGACGTAAACGGC |
| | CACAAGTTCAGCGTGTCTGGCGAGGGCGAGGGCGATGCCACCTACGGCAAGC |
| | TGACCCTGAAGTTCATCTGCACCACCGGCAAGCTGCCCGTGCCCTGGCCCAC |
| | CCTCGTGACCACCCTGACCTACGGCGTGCAGTGCTTCAGCCGCTACCCCGAC |
| | CACATGAAGCAGCACGACTTCTTCAAGTCCGCCATGCCCGAAGGCTACGTCC |
| | AGGAGCGCACCATCTTCTTCAAGGACGACGGCAACTACAAGACCCGCGCCGA |
| | GGTGAAGTTCGAGGGCGACACCCTGGTGAACCGCATCGAGCTGAAGGGCATC |
| | GACTTCAAGGAGGACGGCAACATCCTGGGGCACAAGCTGGAGTACAACTACA |
| | ACAGCCACAACGTCTATATCATGGCCGACAAGCAGAAGAACGGCATCAAGGC |
| | GAACTTCAAGATCCGCCACAACATCGAGGACGGCAGCGTGCAGCTCGCCGAC |
| | CACTACCAGCAGAACACCCCCATCGGCGACGGCCCCGTGCTGCTGCCCGACA |
| | ACCACTACCTGAGCACCCAGTCCGCCCTGAGCAAAGACCCCAACGAGAAGCG |
| | CGATCACATGGTCCTGCTGGAGTTCGTGACCGCCGCCGGGATCACTCTCGGC |
| | ATGGACGAGCTGTACAAGTAATTAAAACAGCCTGTGGGTTGATCCCACCCAC |
| | AGGCCCATTGGGCGCTAGCACTCTGGTATCACGGTACCTTTGTGCGCCTGTT |
| | TTATACCCCCTCCCCCAACTGTAACTTAGAAGTAACACACACCGATCAACAG |
| | TCAGCGTGGCACACCAGCCACGTTTTGATCAAGCACTTCTGTTACCCCGGAC |
| | TGAGTATCAATAGACTGCTCACGCGGTTGAAGGAGAAAGCGTTCGTTATCCG |
| | GCCAACTACTTCGAAAAACCTAGTAACACCGTGGAAGTTGCAGAGTGTTTCG |
| | CTCAGCACTACCCCAGTGTAGATCAGGTCGATGAGTCACCGCATTCCCCACG |
| | GGCGACCGTGGCGGTGGCTGCGTTGGCGGCCTGCCCATGGGGAAACCCATGG |
| | GACGCTCTAATACAGACATGGTGCGAAGAGTCTATTGAGCTAGTTGGTAGTC |
| | CTCCGGCCCCTGAATGCGGCTAATCCTAACTGCGGAGCACACACCCTCAAGC |
| | CAGAGGGCAGTGTGTCGTAACGGGCAACTCTGCAGCGGAACCGACTACTTTG |
| | GGTGTCCGTGTTTCATTTTATTCCTATACTGGCTGCTTATGGTGACAATTGA |
| | GAGATCGTTACCATATAGCTATTGGATTGGCCATCCGGTGACTAATAGAGCT |
| | ATTATATATCCCTTTGTTGGGTTTATACCACTTAGCTTGAAAGAGGTTAAAA |
| | CATTACAATTCATTGTTAAGTTGAATACAGCAAAAAAAAACAAAAAACAAAA |
| | CGGCTATTATGCGTTACCGGCGAGACGCTACGGACTTAAATAATTGAGCCTT |
| | AAAGAAGAAATTCTTTAAGTGGATGCTCTCAAACTCAGGGAAACCTAAATCT |
| | AGTTATAGACAAGGCAATCCTGAGCCAAGCCGAAGTAGTAATTAGTAAGACC |
| | AGTGGACAATCGACG |
| SEQ ID NO. | TAATACGACTCACTATAGGGGATCCGGGAGACCCTCGACCGTCGATTGTCCA |
| 33: | CTGGTCAACAATAGATGACTTACAACTAATCGGAAGGTGCAGAGACTCGACG |
| pML78_EGF | GGAGCTACCCTAACGTCAAGACGAGGGTAAAGAGAGAGTCCAATTCTCAAAG |
| P-CVB3- | CCAATAGGCAGTAGCGAAAGCTGCAAGAGAATGAAAATCCGTCGCCGGAAAC |
| | GCAATAGCCGACAAAAAACAAAAAAAACAAAAAAAAAACCAAAAAAACAAAA |
| | CACAATGGTGAGCAAGGGCGAGGAGCTGTTCACCGGGGTGGTGCCCATCCTG |
| | GTCGAGCTGGACGGCGACGTAAACGGCCACAAGTTCAGCGTGTCTGGCGAGG |
| | GCGAGGGCGATGCCACCTACGGCAAGCTGACCCTGAAGTTCATCTGCACCAC |
| | CGGCAAGCTGCCCGTGCCCTGGCCCACCCTCGTGACCACCCTGACCTACGGC |
| | GTGCAGTGCTTCAGCCGCTACCCCGACCACATGAAGCAGCACGACTTCTTCA |
| | AGTCCGCCATGCCCGAAGGCTACGTCCAGGAGCGCACCATCTTCTTCAAGGA |
| | CGACGGCAACTACAAGACCCGCGCCGAGGTGAAGTTCGAGGGCGACACCCTG |
| | GTGAACCGCATCGAGCTGAAGGGCATCGACTTCAAGGAGGACGGCAACATCC |
| | TGGGGCACAAGCTGGAGTACAACTACAACAGCCACAACGTCTATATCATGGC |
| | CGACAAGCAGAAGAACGGCATCAAGGCGAACTTCAAGATCCGCCACAACATC |
| | GAGGACGGCAGCGTGCAGCTCGCCGACCACTACCAGCAGAACACCCCCATCG |
| | GCGACGGCCCCGTGCTGCTGCCCGACAACCACTACCTGAGCACCCAGTCCGC |
| | CCTGAGCAAAGACCCCAACGAGAAGCGCGATCACATGGTCCTGCTGGAGTTC |
| | GTGACCGCCGCCGGGATCACTCTCGGCATGGACGAGCTGTACAAGTAATTAA |
| | AACAGCCTGTGGGTTGATCCCACCCACAGGCCCATTGGGCGCTAGCACTCTG |
| | GTATCACGGTACCTTTGTGCGCCTGTTTTATACCCCCTCCCCCAACTGTAAC |
| | TTAGAAGTAACACACACCGATCAACAGTCAGCGTGGCACACCAGCCACGTTT |
| | TGATCAAGCACTTCTGTTACCCCGGACTGAGTATCAATAGACTGCTCACGCG |
| | GTTGAAGGAGAAAGCGTTCGTTATCCGGCCAACTACTTCGAAAAACCTAGTA |
| | ACACCGTGGAAGTTGCAGAGTGTTTCGCTCAGCACTACCCCAGTGTAGATCA |
| | GGTCGATGAGTCACCGCATTCCCCACGGGCGACCGTGGCGGTGGCTGCGTTG |
| | GCGGCCTGCCCATGGGGAAACCCATGGGACGCTCTAATACAGACATGGTGCG |
| | AAGAGTCTATTGAGCTAGTTGGTAGTCCTCCGGCCCCTGAATGCGGCTAATC |
| | CTAACTGCGGAGCACACACCCTCAAGCCAGAGGGCAGTGTGTCGTAACGGGC |
| | AACTCTGCAGCGGAACCGACTACTTTGGGTGTCCGTGTTTCATTTTATTCCT |
| | ATACTGGCTGCTTATGGTGACAATTGAGAGATCGTTACCATATAGCTATTGG |
| | ATTGGCCATCCGGTGACTAATAGAGCTATTATATATCCCTTTGTTGGGTTTA |
| | TACCACTTAGCTTGAAAGAGGTTAAAACATTACAATTCATTGTTAAGTTGAA |
| | TACAGCAAAAAAAAACAAAAAACAAAACGGCTATTATGCGTTACCGGCGACG |
| | GACTTAAATAATTGAGCCTTAAAGAAGAAATTCTTTAAGTGGATGCTCTCAA |
| | ACTCAGGGAAACCTAAATCTAGTTATAGACAAGGCAATCCTGAGCCAAGCCG |
| | AAGTAGTAATTAGTAAGACCAGTGGACAATCGACG |
| SEQ ID NO. | TAATACGACTCACTATAGGGGATCCGGGAGACCCTCGACCGTCGATTGTCCA |
| 34: | CTGGTCAACAATAGATGACTTACAACTAATCGGAAGGTGCAGAGACTCGACG |
| pML79_EGF | GGAGCTACCCTAACGTCAAGACGAGGGTAAAGAGAGAGTCCAATTCTCAAAG |
| P-CVB3- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | ACAAAAAACAAAAAAAACAAAAAAAAAACCAAAAAAACAAAACACAATGGTG |
| | AGCAAGGGCGAGGAGCTGTTCACCGGGGTGGTGCCCATCCTGGTCGAGCTGG |
| | ACGGCGACGTAAACGGCCACAAGTTCAGCGTGTCTGGCGAGGGCGAGGGCGA |
| | TGCCACCTACGGCAAGCTGACCCTGAAGTTCATCTGCACCACCGGCAAGCTG |
| | CCCGTGCCCTGGCCCACCCTCGTGACCACCCTGACCTACGGCGTGCAGTGCT |
| | TCAGCCGCTACCCCGACCACATGAAGCAGCACGACTTCTTCAAGTCCGCCAT |
| | GCCCGAAGGCTACGTCCAGGAGCGCACCATCTTCTTCAAGGACGACGGCAAC |
| | TACAAGACCCGCGCCGAGGTGAAGTTCGAGGGCGACACCCTGGTGAACCGCA |
| | TCGAGCTGAAGGGCATCGACTTCAAGGAGGACGGCAACATCCTGGGGCACAA |
| | GCTGGAGTACAACTACAACAGCCACAACGTCTATATCATGGCCGACAAGCAG |
| | AAGAACGGCATCAAGGCGAACTTCAAGATCCGCCACAACATCGAGGACGGCA |
| | GCGTGCAGCTCGCCGACCACTACCAGCAGAACACCCCCATCGGCGACGGCCC |
| | CGTGCTGCTGCCCGACAACCACTACCTGAGCACCCAGTCCGCCCTGAGCAAA |
| | GACCCCAACGAGAAGCGCGATCACATGGTCCTGCTGGAGTTCGTGACCGCCG |
| | CCGGGATCACTCTCGGCATGGACGAGCTGTACAAGTAATTAAAACAGCCTGT |
| | GGGTTGATCCCACCCACAGGCCCATTGGGCGCTAGCACTCTGGTATCACGGT |
| | ACCTTTGTGCGCCTGTTTTATACCCCCTCCCCCAACTGTAACTTAGAAGTAA |
| | CACACACCGATCAACAGTCAGCGTGGCACACCAGCCACGTTTTGATCAAGCA |
| | CTTCTGTTACCCCGGACTGAGTATCAATAGACTGCTCACGCGGTTGAAGGAG |
| | AAAGCGTTCGTTATCCGGCCAACTACTTCGAAAAACCTAGTAACACCGTGGA |
| | AGTTGCAGAGTGTTTCGCTCAGCACTACCCCAGTGTAGATCAGGTCGATGAG |
| | TCACCGCATTCCCCACGGGCGACCGTGGCGGTGGCTGCGTTGGCGGCCTGCC |
| | CATGGGGAAACCCATGGGACGCTCTAATACAGACATGGTGCGAAGAGTCTAT |
| | TGAGCTAGTTGGTAGTCCTCCGGCCCCTGAATGCGGCTAATCCTAACTGCGG |
| | AGCACACACCCTCAAGCCAGAGGGCAGTGTGTCGTAACGGGCAACTCTGCAG |
| | CGGAACCGACTACTTTGGGTGTCCGTGTTTCATTTTATTCCTATACTGGCTG |
| | CTTATGGTGACAATTGAGAGATCGTTACCATATAGCTATTGGATTGGCCATC |
| | CGGTGACTAATAGAGCTATTATATATCCCTTTGTTGGGTTTATACCACTTAG |
| | CTTGAAAGAGGTTAAAACATTACAATTCATTGTTAAGTTGAATACAGCAAAA |
| | AAAAACAAAAAACAAAACAAAAAAAAAAAAAGCCCGGATAGCTCAGTCGGTA |
| | GAGCAGCGGCCTATGCGGCCGCCGACCAGAATCATGCAAGTGCGTAAGATAG |
| | TCGCGGGTCGGCGGCCGCATTCGAGGCCGCGTCCAGGGTTCAAGTCCCTGTT |
| | CGGGCGCCACTGCAGAAAAAAAAAAAAGGCTATTATGCGTTACCGGCGAGAC |
| | GCTACGGACTTAAATAATTGAGCCTTAAAGAAGAAATTCTTTAAGTGGATGC |
| | TCTCAAACTCAGGGAAACCTAAATCTAGTTATAGACAAGGCAATCCTGAGCC |
| | AAGCCGAAGTAGTAATTAGTAAGACCAGTGGACAATCGACG |
| SEQ ID NO. | TAATACGACTCACTATAGGGGATCCGGGAGACCCTCGACCGTCGATTGTCCA |
| 35: | CTGGTCAACAATAGATGACTTACAACTAATCGGAAGGTGCAGAGACTCGACG |
| pML80_EGF | GGAGCTACCCTAACGTCAAGACGAGGGTAAAGAGAGAGTCCAATTCTCAAAG |
| P-CVB3- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | ACAAAAAACAAAAAAAACAAAAAAAAAACCAAAAAAACAAAACACAATGGTG |
| | AGCAAGGGCGAGGAGCTGTTCACCGGGGTGGTGCCCATCCTGGTCGAGCTGG |
| | ACGGCGACGTAAACGGCCACAAGTTCAGCGTGTCTGGCGAGGGCGAGGGCGA |
| | TGCCACCTACGGCAAGCTGACCCTGAAGTTCATCTGCACCACCGGCAAGCTG |
| | CCCGTGCCCTGGCCCACCCTCGTGACCACCCTGACCTACGGCGTGCAGTGCT |
| | TCAGCCGCTACCCCGACCACATGAAGCAGCACGACTTCTTCAAGTCCGCCAT |
| | GCCCGAAGGCTACGTCCAGGAGCGCACCATCTTCTTCAAGGACGACGGCAAC |
| | TACAAGACCCGCGCCGAGGTGAAGTTCGAGGGCGACACCCTGGTGAACCGCA |
| | TCGAGCTGAAGGGCATCGACTTCAAGGAGGACGGCAACATCCTGGGGCACAA |
| | GCTGGAGTACAACTACAACAGCCACAACGTCTATATCATGGCCGACAAGCAG |
| | AAGAACGGCATCAAGGCGAACTTCAAGATCCGCCACAACATCGAGGACGGCA |
| | GCGTGCAGCTCGCCGACCACTACCAGCAGAACACCCCCATCGGCGACGGCCC |
| | CGTGCTGCTGCCCGACAACCACTACCTGAGCACCCAGTCCGCCCTGAGCAAA |
| | GACCCCAACGAGAAGCGCGATCACATGGTCCTGCTGGAGTTCGTGACCGCCG |
| | CCGGGATCACTCTCGGCATGGACGAGCTGTACAAGTAATTAAAACAGCCTGT |
| | GGGTTGATCCCACCCACAGGCCCATTGGGCGCTAGCACTCTGGTATCACGGT |
| | ACCTTTGTGCGCCTGTTTTATACCCCCTCCCCCAACTGTAACTTAGAAGTAA |
| | CACACACCGATCAACAGTCAGCGTGGCACACCAGCCACGTTTTGATCAAGCA |
| | CTTCTGTTACCCCGGACTGAGTATCAATAGACTGCTCACGCGGTTGAAGGAG |
| | AAAGCGTTCGTTATCCGGCCAACTACTTCGAAAAACCTAGTAACACCGTGGA |
| | AGTTGCAGAGTGTTTCGCTCAGCACTACCCCAGTGTAGATCAGGTCGATGAG |
| | TCACCGCATTCCCCACGGGCGACCGTGGCGGTGGCTGCGTTGGCGGCCTGCC |
| | CATGGGGAAACCCATGGGACGCTCTAATACAGACATGGTGCGAAGAGTCTAT |
| | TGAGCTAGTTGGTAGTCCTCCGGCCCCTGAATGCGGCTAATCCTAACTGCGG |
| | AGCACACACCCTCAAGCCAGAGGGCAGTGTGTCGTAACGGGCAACTCTGCAG |
| | CGGAACCGACTACTTTGGGTGTCCGTGTTTCATTTTATTCCTATACTGGCTG |
| | CTTATGGTGACAATTGAGAGATCGTTACCATATAGCTATTGGATTGGCCATC |
| | CGGTGACTAATAGAGCTATTATATATCCCTTTGTTGGGTTTATACCACTTAG |
| | CTTGAAAGAGGTTAAAACATTACAATTCATTGTTAAGTTGAATACAGCAAAA |
| | AAAAAAAAAAAAGCCCGGATAGCTCAGTCGGTAGAGCAGCGGCCTATGCGGC |
| | CGCCGACCAGAATCATGCAAGTGCGTAAGATAGTCGCGGGTCGGCGGCCGCA |
| | TTCGAGGCCGCGTCCAGGGTTCAAGTCCCTGTTCGGGCGCCACTGCAGAAAA |
| | AAAAAAAAAAAAAACAAAAAACAAAACGGCTATTATGCGTTACCGGCGAGAC |
| | GCTACGGACTTAAATAATTGAGCCTTAAAGAAGAAATTCTTTAAGTGGATGC |
| | TCTCAAACTCAGGGAAACCTAAATCTAGTTATAGACAAGGCAATCCTGAGCC |
| | AAGCCGAAGTAGTAATTAGTAAGACCAGTGGACAATCGACG |
| SEQ ID NO. | TAATACGACTCACTATAGGGGATCCGGGAGACCCTCGACCGTCGATTGTCCA |
| 36: | CTGGTCAACAATAGATGACTTACAACTAATCGGAAGGTGCAGAGACTCGACG |
| pML81_EGF | GGAGCTACCCTAACGTCAAGACGAGGGTAAAGAGAGAGTCCAATTCTCAAAG |
| P-CVB3- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | CAAAAAACAAAAAAAACAAAAAAAAAACCAAAAAAACAAAACACAATGGTGA |
| | GCAAGGGCGAGGAGCTGTTCACCGGGGTGGTGCCCATCCTGGTCGAGCTGGA |
| | CGGCGACGTAAACGGCCACAAGTTCAGCGTGTCTGGCGAGGGCGAGGGCGAT |
| | GCCACCTACGGCAAGCTGACCCTGAAGTTCATCTGCACCACCGGCAAGCTGC |
| | CCGTGCCCTGGCCCACCCTCGTGACCACCCTGACCTACGGCGTGCAGTGCTT |
| | CAGCCGCTACCCCGACCACATGAAGCAGCACGACTTCTTCAAGTCCGCCATG |
| | CCCGAAGGCTACGTCCAGGAGCGCACCATCTTCTTCAAGGACGACGGCAACT |
| | ACAAGACCCGCGCCGAGGTGAAGTTCGAGGGCGACACCCTGGTGAACCGCAT |
| | CGAGCTGAAGGGCATCGACTTCAAGGAGGACGGCAACATCCTGGGGCACAAG |
| | CTGGAGTACAACTACAACAGCCACAACGTCTATATCATGGCCGACAAGCAGA |
| | AGAACGGCATCAAGGCGAACTTCAAGATCCGCCACAACATCGAGGACGGCAG |
| | CGTGCAGCTCGCCGACCACTACCAGCAGAACACCCCCATCGGCGACGGCCCC |
| | GTGCTGCTGCCCGACAACCACTACCTGAGCACCCAGTCCGCCCTGAGCAAAG |
| | ACCCCAACGAGAAGCGCGATCACATGGTCCTGCTGGAGTTCGTGACCGCCGC |
| | CGGGATCACTCTCGGCATGGACGAGCTGTACAAGTAATTAAAACAGCCTGTG |
| | GGTTGATCCCACCCACAGGCCCATTGGGCGCTAGCACTCTGGTATCACGGTA |
| | CCTTTGTGCGCCTGTTTTATACCCCCTCCCCCAACTGTAACTTAGAAGTAAC |
| | ACACACCGATCAACAGTCAGCGTGGCACACCAGCCACGTTTTGATCAAGCAC |
| | TTCTGTTACCCCGGACTGAGTATCAATAGACTGCTCACGCGGTTGAAGGAGA |
| | AAGCGTTCGTTATCCGGCCAACTACTTCGAAAAACCTAGTAACACCGTGGAA |
| | GTTGCAGAGTGTTTCGCTCAGCACTACCCCAGTGTAGATCAGGTCGATGAGT |
| | CACCGCATTCCCCACGGGCGACCGTGGCGGTGGCTGCGTTGGCGGCCTGCCC |
| | ATGGGGAAACCCATGGGACGCTCTAATACAGACATGGTGCGAAGAGTCTATT |
| | GAGCTAGTTGGTAGTCCTCCGGCCCCTGAATGCGGCTAATCCTAACTGCGGA |
| | GCACACACCCTCAAGCCAGAGGGCAGTGTGTCGTAACGGGCAACTCTGCAGC |
| | GGAACCGACTACTTTGGGTGTCCGTGTTTCATTTTATTCCTATACTGGCTGC |
| | TTATGGTGACAATTGAGAGATCGTTACCATATAGCTATTGGATTGGCCATCC |
| | GGTGACTAATAGAGCTATTATATATCCCTTTGTTGGGTTTATACCACTTAGC |
| | TTGAAAGAGGTTAAAACATTACAATTCATTGTTAAGTTGAATACAGCAAAAA |
| | AAAACAAAAAACAAAACGGCTATTATGCGTTACCGGCGAGACGCTACGGACT |
| | TAAATAATTGAGCCTTAAAGAAGAAATTCTTTAAGTGGATGCTCTCAAACTC |
| | AGGGAAACCTAAATCTAGTTATAGACAAGGCAATCCTGAGCCAAGCCGAAGT |
| | AGTAATTAGTAAGACCAGTGGACAATCGACGAAAAAAAAAAAAAGCCCGGAT |
| | AGCTCAGTCGGTAGAGCAGCGGCCTATGCGGCCGCCGACCAGAATCATGCAA |
| | GTGCGTAAGATAGTCGCGGGTCGGCGGCCGCATTCGAGGCCGCGTCCAGGGT |
| | TCAAGTCCCTGTTCGGGCGCCA |
| SEQ ID NO. | TAATACGACTCACTATAGGGGATCCGGGAGACCCTCGACCGTCGATTGTCCA |
| 37: | CTGGTCAACAATAGATGACTTACAACTAATCGGAAGGTGCAGAGACTCGACG |
| pML82_EGF | GGAGCTACCCTAACGTCAAGACGAGGGTAAAGAGAGAGTCCAATTCTCAAAG |
| P-CVB3- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | CAAAAAACAAAAAAAACAAAAAAAAAACCAAAAAAACAAAACACAATGGTGA |
| | GCAAGGGCGAGGAGCTGTTCACCGGGGTGGTGCCCATCCTGGTCGAGCTGGA |
| | CGGCGACGTAAACGGCCACAAGTTCAGCGTGTCTGGCGAGGGCGAGGGCGAT |
| | GCCACCTACGGCAAGCTGACCCTGAAGTTCATCTGCACCACCGGCAAGCTGC |
| | CCGTGCCCTGGCCCACCCTCGTGACCACCCTGACCTACGGCGTGCAGTGCTT |
| | CAGCCGCTACCCCGACCACATGAAGCAGCACGACTTCTTCAAGTCCGCCATG |
| | CCCGAAGGCTACGTCCAGGAGCGCACCATCTTCTTCAAGGACGACGGCAACT |
| | ACAAGACCCGCGCCGAGGTGAAGTTCGAGGGCGACACCCTGGTGAACCGCAT |
| | CGAGCTGAAGGGCATCGACTTCAAGGAGGACGGCAACATCCTGGGGCACAAG |
| | CTGGAGTACAACTACAACAGCCACAACGTCTATATCATGGCCGACAAGCAGA |
| | AGAACGGCATCAAGGCGAACTTCAAGATCCGCCACAACATCGAGGACGGCAG |
| | CGTGCAGCTCGCCGACCACTACCAGCAGAACACCCCCATCGGCGACGGCCCC |
| | GTGCTGCTGCCCGACAACCACTACCTGAGCACCCAGTCCGCCCTGAGCAAAG |
| | ACCCCAACGAGAAGCGCGATCACATGGTCCTGCTGGAGTTCGTGACCGCCGC |
| | CGGGATCACTCTCGGCATGGACGAGCTGTACAAGTAATTAAAACAGCCTGTG |
| | GGTTGATCCCACCCACAGGCCCATTGGGCGCTAGCACTCTGGTATCACGGTA |
| | CCTTTGTGCGCCTGTTTTATACCCCCTCCCCCAACTGTAACTTAGAAGTAAC |
| | ACACACCGATCAACAGTCAGCGTGGCACACCAGCCACGTTTTGATCAAGCAC |
| | TTCTGTTACCCCGGACTGAGTATCAATAGACTGCTCACGCGGTTGAAGGAGA |
| | AAGCGTTCGTTATCCGGCCAACTACTTCGAAAAACCTAGTAACACCGTGGAA |
| | GTTGCAGAGTGTTTCGCTCAGCACTACCCCAGTGTAGATCAGGTCGATGAGT |
| | CACCGCATTCCCCACGGGCGACCGTGGCGGTGGCTGCGTTGGCGGCCTGCCC |
| | ATGGGGAAACCCATGGGACGCTCTAATACAGACATGGTGCGAAGAGTCTATT |
| | GAGCTAGTTGGTAGTCCTCCGGCCCCTGAATGCGGCTAATCCTAACTGCGGA |
| | GCACACACCCTCAAGCCAGAGGGCAGTGTGTCGTAACGGGCAACTCTGCAGC |
| | GGAACCGACTACTTTGGGTGTCCGTGTTTCATTTTATTCCTATACTGGCTGC |
| | TTATGGTGACAATTGAGAGATCGTTACCATATAGCTATTGGATTGGCCATCC |
| | GGTGACTAATAGAGCTATTATATATCCCTTTGTTGGGTTTATACCACTTAGC |
| | TTGAAAGAGGTTAAAACATTACAATTCATTGTTAAGTTGAATACAGCAAAAA |
| | AAAACAAAAAACAAAACGGCTATTATGCGTTACCGGCGAGACGCTACGGACT |
| | TAAATAATTGAGCCTTAAAGAAGAAATTCTTTAAGTGGATGCTCTCAAACTC |
| | AGGGAAACCTAAATCTAGTTATAGACAAGGCAATCCTGAGCCAAGCCGAAGT |
| | AGTAATTAGTAAAAAAAAAAAAAAAGCCCGGATAGCTCAGTCGGTAGAGCAG |
| | CGGCCTATGCGGCCGCCGACCAGAATCATGCAAGTGCGTAAGATAGTCGCGG |
| | GTCGGCGGCCGCATTCGAGGCCGCGTCCAGGGTTCAAGTCCCTGTTCGGGCG |
| | CCACTGCAGAAAAAAAAAAAAGACCAGTGGACAATCGACG |
| SEQ ID NO. | TAATACGACTCACTATAGGGGATCCGGGAGACCCTCGACAAAAAAAAAAAAA |
| 38: | GCCCGGATAGCTCAGTCGGTAGAGCAGCGGCCTATGCGGCCGCCGACCAGAA |
| pML83_tS1m | TCATGCAAGTGCGTAAGATAGTCGCGGGTCGGCGGCCGCATTCGAGGCCGCG |
| -EGFP- | TCCAGGGTTCAAGTCCCTGTTCGGGCGCCACTGCAGAAAAAAAAAAAACGTC |
| | GATTGTCCACTGGTCAACAATAGATGACTTACAACTAATCGGAAGGTGCAGA |
| | GACTCGACGGGAGCTACCCTAACGTCAAGACGAGGGTAAAGAGAGAGTCCAA |
| | TTCTCAAAGCCAATAGGCAGTAGCGAAAGCTGCAAGAGAATGAAAATCCGTT |
| | GACCTTAAACGGTCGTGTGGGTTCAAGTCCCTCCACCCCCACGCCGGAAACG |
| | CAATAGCCGCAAAAAAAAAAAAAACAAAAAAAAAACCAAAAAAACAAAACAC |
| | AATGGTGAGCAAGGGCGAGGAGCTGTTCACCGGGGTGGTGCCCATCCTGGTC |
| | GAGCTGGACGGCGACGTAAACGGCCACAAGTTCAGCGTGTCTGGCGAGGGCG |
| | AGGGCGATGCCACCTACGGCAAGCTGACCCTGAAGTTCATCTGCACCACCGG |
| | CAAGCTGCCCGTGCCCTGGCCCACCCTCGTGACCACCCTGACCTACGGCGTG |
| | CAGTGCTTCAGCCGCTACCCCGACCACATGAAGCAGCACGACTTCTTCAAGT |
| | CCGCCATGCCCGAAGGCTACGTCCAGGAGCGCACCATCTTCTTCAAGGACGA |
| | CGGCAACTACAAGACCCGCGCCGAGGTGAAGTTCGAGGGCGACACCCTGGTG |
| | AACCGCATCGAGCTGAAGGGCATCGACTTCAAGGAGGACGGCAACATCCTGG |
| | GGCACAAGCTGGAGTACAACTACAACAGCCACAACGTCTATATCATGGCCGA |
| | CAAGCAGAAGAACGGCATCAAGGCGAACTTCAAGATCCGCCACAACATCGAG |
| | GACGGCAGCGTGCAGCTCGCCGACCACTACCAGCAGAACACCCCCATCGGCG |
| | ACGGCCCCGTGCTGCTGCCCGACAACCACTACCTGAGCACCCAGTCCGCCCT |
| | GAGCAAAGACCCCAACGAGAAGCGCGATCACATGGTCCTGCTGGAGTTCGTG |
| | ACCGCCGCCGGGATCACTCTCGGCATGGACGAGCTGTACAAGTAATTAAAAC |
| | AGCCTGTGGGTTGATCCCACCCACAGGCCCATTGGGCGCTAGCACTCTGGTA |
| | TCACGGTACCTTTGTGCGCCTGTTTTATACCCCCTCCCCCAACTGTAACTTA |
| | GAAGTAACACACACCGATCAACAGTCAGCGTGGCACACCAGCCACGTTTTGA |
| | TCAAGCACTTCTGTTACCCCGGACTGAGTATCAATAGACTGCTCACGCGGTT |
| | GAAGGAGAAAGCGTTCGTTATCCGGCCAACTACTTCGAAAAACCTAGTAACA |
| | CCGTGGAAGTTGCAGAGTGTTTCGCTCAGCACTACCCCAGTGTAGATCAGGT |
| | CGATGAGTCACCGCATTCCCCACGGGCGACCGTGGCGGTGGCTGCGTTGGCG |
| | GCCTGCCCATGGGGAAACCCATGGGACGCTCTAATACAGACATGGTGCGAAG |
| | AGTCTATTGAGCTAGTTGGTAGTCCTCCGGCCCCTGAATGCGGCTAATCCTA |
| | ACTGCGGAGCACACACCCTCAAGCCAGAGGGCAGTGTGTCGTAACGGGCAAC |
| | TCTGCAGCGGAACCGACTACTTTGGGTGTCCGTGTTTCATTTTATTCCTATA |
| | CTGGCTGCTTATGGTGACAATTGAGAGATCGTTACCATATAGCTATTGGATT |
| | GGCCATCCGGTGACTAATAGAGCTATTATATATCCCTTTGTTGGGTTTATAC |
| | CACTTAGCTTGAAAGAGGTTAAAACATTACAATTCATTGTTAAGTTGAATAC |
| | AGCAAAAAAAAACAAAAAACAAAACGGCTATTATGCGTTACCGGCGAGACGC |
| | TACGGACTTAAATAATTGAGCCTTAAAGAAGAAATTCTTTAAGTGGATGCTC |
| | TCAAACTCAGGGAAACCTAAATCTAGTTATAGACAAGGCAATCCTGAGCCAA |
| | GCCGAAGTAGTAATTAGTAAGACCAGTGGACAATCGACG |
| SEQ ID NO. | TAATACGACTCACTATAGGGGATCCGGGAGACCCTCGACCGTCGATTGTCCA |
| 39: | CTGGTCAAAAAAAAAAAAAGCCCGGATAGCTCAGTCGGTAGAGCAGCGGCCT |
| pML84_tS1m | ATGCGGCCGCCGACCAGAATCATGCAAGTGCGTAAGATAGTCGCGGGTCGGC |
| -EGFP- | GGCCGCATTCGAGGCCGCGTCCAGGGTTCAAGTCCCTGTTCGGGCGCCACTG |
| | CAGAAAAAAAAAAAAAACAATAGATGACTTACAACTAATCGGAAGGTGCAGA |
| | GACTCGACGGGAGCTACCCTAACGTCAAGACGAGGGTAAAGAGAGAGTCCAA |
| | TTCTCAAAGCCAATAGGCAGTAGCGAAAGCTGCAAGAGAATGAAAATCCGTT |
| | GACCTTAAACGGTCGTGTGGGTTCAAGTCCCTCCACCCCCACGCCGGAAACG |
| | CAATAGCCGCAAAAAACAAAAAAAAAAAAAAAAAACCAAAAAAACAAAACAC |
| | AATGGTGAGCAAGGGCGAGGAGCTGTTCACCGGGGTGGTGCCCATCCTGGTC |
| | GAGCTGGACGGCGACGTAAACGGCCACAAGTTCAGCGTGTCTGGCGAGGGCG |
| | AGGGCGATGCCACCTACGGCAAGCTGACCCTGAAGTTCATCTGCACCACCGG |
| | CAAGCTGCCCGTGCCCTGGCCCACCCTCGTGACCACCCTGACCTACGGCGTG |
| | CAGTGCTTCAGCCGCTACCCCGACCACATGAAGCAGCACGACTTCTTCAAGT |
| | CCGCCATGCCCGAAGGCTACGTCCAGGAGCGCACCATCTTCTTCAAGGACGA |
| | CGGCAACTACAAGACCCGCGCCGAGGTGAAGTTCGAGGGCGACACCCTGGTG |
| | AACCGCATCGAGCTGAAGGGCATCGACTTCAAGGAGGACGGCAACATCCTGG |
| | GGCACAAGCTGGAGTACAACTACAACAGCCACAACGTCTATATCATGGCCGA |
| | CAAGCAGAAGAACGGCATCAAGGCGAACTTCAAGATCCGCCACAACATCGAG |
| | GACGGCAGCGTGCAGCTCGCCGACCACTACCAGCAGAACACCCCCATCGGCG |
| | ACGGCCCCGTGCTGCTGCCCGACAACCACTACCTGAGCACCCAGTCCGCCCT |
| | GAGCAAAGACCCCAACGAGAAGCGCGATCACATGGTCCTGCTGGAGTTCGTG |
| | ACCGCCGCCGGGATCACTCTCGGCATGGACGAGCTGTACAAGTAATTAAAAC |
| | AGCCTGTGGGTTGATCCCACCCACAGGCCCATTGGGCGCTAGCACTCTGGTA |
| | TCACGGTACCTTTGTGCGCCTGTTTTATACCCCCTCCCCCAACTGTAACTTA |
| | GAAGTAACACACACCGATCAACAGTCAGCGTGGCACACCAGCCACGTTTTGA |
| | TCAAGCACTTCTGTTACCCCGGACTGAGTATCAATAGACTGCTCACGCGGTT |
| | GAAGGAGAAAGCGTTCGTTATCCGGCCAACTACTTCGAAAAACCTAGTAACA |
| | CCGTGGAAGTTGCAGAGTGTTTCGCTCAGCACTACCCCAGTGTAGATCAGGT |
| | CGATGAGTCACCGCATTCCCCACGGGCGACCGTGGCGGTGGCTGCGTTGGCG |
| | GCCTGCCCATGGGGAAACCCATGGGACGCTCTAATACAGACATGGTGCGAAG |
| | AGTCTATTGAGCTAGTTGGTAGTCCTCCGGCCCCTGAATGCGGCTAATCCTA |
| | ACTGCGGAGCACACACCCTCAAGCCAGAGGGCAGTGTGTCGTAACGGGCAAC |
| | TCTGCAGCGGAACCGACTACTTTGGGTGTCCGTGTTTCATTTTATTCCTATA |
| | CTGGCTGCTTATGGTGACAATTGAGAGATCGTTACCATATAGCTATTGGATT |
| | GGCCATCCGGTGACTAATAGAGCTATTATATATCCCTTTGTTGGGTTTATAC |
| | CACTTAGCTTGAAAGAGGTTAAAACATTACAATTCATTGTTAAGTTGAATAC |
| | AGCAAAAAAAAACAAAAAACAAAACGGCTATTATGCGTTACCGGCGAGACGC |
| | TACGGACTTAAATAATTGAGCCTTAAAGAAGAAATTCTTTAAGTGGATGCTC |
| | TCAAACTCAGGGAAACCTAAATCTAGTTATAGACAAGGCAATCCTGAGCCAA |
| | GCCGAAGTAGTAATTAGTAAAAAAAAAAAAAAAGCCCGGATAGCTCAGTCGG |
| | TAGAGCAGCGGCCTATGCGGCCGCCGACCAGAATCATGCAAGTGCGTAAGAT |
| | AGTCGCGGGTCGGCGGCCGCATTCGAGGCCGCGTCCAGGGTTCAAGTCCCTG |
| | TTCGGGCGCCACTGCAGAAAAAAAAAAAAGACCAGTGGACAATCGACG |
| SEQ ID NO. | TAATACGACTCACTATAGGGGATCCGGGAGACCCTCGACAAAAAAAAAAAAA |
| 40: | GCCCGGATAGCTCAGTCGGTAGAGCAGCGGCCTATGCGGCCGCCGACCAGAA |
| pML85_tS1m | TCATGCAAGTGCGTAAGATAGTCGCGGGTCGGCGGCCGCATTCGAGGCCGCG |
| -EGFP- | TCCAGGGTTCAAGTCCCTGTTCGGGCGCCACTGCAGAAAAAAAAAAAACGTC |
| | GATTGTCCACTGGTCAACAATAGATGACTTACAACTAATCGGAAGGTGCAGA |
| | GACTCGACGGGAGCTACCCTAACGTCAAGACGAGGGTAAAGAGAGAGTCCAA |
| | TTCTCAAAGCCAATAGGCAGTAGCGAAAGCTGCAAGAGAATGAAAATCCGTT |
| | GACCTTAAACGGTCGTGTGGGTTCAAGTCCCTCCACCCCCACGCCGGAAACG |
| | CAATAGCCGCAAAAAACAAAAAAAACAAAAAAAAAACCAAAAAAACAAAACA |
| | CAATGGTGAGCAAGGGCGAGGAGCTGTTCACCGGGGTGGTGCCCATCCTGGT |
| | CGAGCTGGACGGCGACGTAAACGGCCACAAGTTCAGCGTGTCTGGCGAGGGC |
| | GAGGGCGATGCCACCTACGGCAAGCTGACCCTGAAGTTCATCTGCACCACCG |
| | GCAAGCTGCCCGTGCCCTGGCCCACCCTCGTGACCACCCTGACCTACGGCGT |
| | GCAGTGCTTCAGCCGCTACCCCGACCACATGAAGCAGCACGACTTCTTCAAG |
| | TCCGCCATGCCCGAAGGCTACGTCCAGGAGCGCACCATCTTCTTCAAGGACG |
| | ACGGCAACTACAAGACCCGCGCCGAGGTGAAGTTCGAGGGCGACACCCTGGT |
| | GAACCGCATCGAGCTGAAGGGCATCGACTTCAAGGAGGACGGCAACATCCTG |
| | GGGCACAAGCTGGAGTACAACTACAACAGCCACAACGTCTATATCATGGCCG |
| | ACAAGCAGAAGAACGGCATCAAGGCGAACTTCAAGATCCGCCACAACATCGA |
| | GGACGGCAGCGTGCAGCTCGCCGACCACTACCAGCAGAACACCCCCATCGGC |
| | GACGGCCCCGTGCTGCTGCCCGACAACCACTACCTGAGCACCCAGTCCGCCC |
| | TGAGCAAAGACCCCAACGAGAAGCGCGATCACATGGTCCTGCTGGAGTTCGT |
| | GACCGCCGCCGGGATCACTCTCGGCATGGACGAGCTGTACAAGTAATTAAAA |
| | CAGCCTGTGGGTTGATCCCACCCACAGGCCCATTGGGCGCTAGCACTCTGGT |
| | ATCACGGTACCTTTGTGCGCCTGTTTTATACCCCCTCCCCCAACTGTAACTT |
| | AGAAGTAACACACACCGATCAACAGTCAGCGTGGCACACCAGCCACGTTTTG |
| | ATCAAGCACTTCTGTTACCCCGGACTGAGTATCAATAGACTGCTCACGCGGT |
| | TGAAGGAGAAAGCGTTCGTTATCCGGCCAACTACTTCGAAAAACCTAGTAAC |
| | ACCGTGGAAGTTGCAGAGTGTTTCGCTCAGCACTACCCCAGTGTAGATCAGG |
| | TCGATGAGTCACCGCATTCCCCACGGGCGACCGTGGCGGTGGCTGCGTTGGC |
| | GGCCTGCCCATGGGGAAACCCATGGGACGCTCTAATACAGACATGGTGCGAA |
| | GAGTCTATTGAGCTAGTTGGTAGTCCTCCGGCCCCTGAATGCGGCTAATCCT |
| | AACTGCGGAGCACACACCCTCAAGCCAGAGGGCAGTGTGTCGTAACGGGCAA |
| | CTCTGCAGCGGAACCGACTACTTTGGGTGTCCGTGTTTCATTTTATTCCTAT |
| | ACTGGCTGCTTATGGTGACAATTGAGAGATCGTTACCATATAGCTATTGGAT |
| | TGGCCATCCGGTGACTAATAGAGCTATTATATATCCCTTTGTTGGGTTTATA |
| | CCACTTAGCTTGAAAGAGGTTAAAACATTACAATTCATTGTTAAGTTGAATA |
| | CAGCAAAAAAAAACAAAAAACAAAACGGCTATTATGCGTTACCGGCGAGACG |
| | CTACGGACTTAAATAATTGAGCCTTAAAGAAGAAATTCTTTAAGTGGATGCT |
| | CTCAAACTCAGGGAAACCTAAATCTAGTTATAGACAAGGCAATCCTGAGCCA |
| | AGCCGAAGTAGTAATTAGTAAGACCAGTGGACAATCGACGAAAAAAAAAAAA |
| | AGCCCGGATAGCTCAGTCGGTAGAGCAGCGGCCTATGCGGCCGCCGACCAGA |
| | ATCATGCAAGTGCGTAAGATAGTCGCGGGTCGGCGGCCGCATTCGAGGCCGC |
| | GTCCAGGGTTCAAGTCCCTGTTCGGGCGCCA |
| SEQ ID NO. | TAATACGACTCACTATAGGGGATCCGGGAGACCCTCGACCGTCGATTGTCCA |
| 41: | CTGGTCAACAATAGATGACTTACAACTAATCGGAAGGTGCAGAGACTCGACG |
| pML86_EGF | GGAGCTACCCTAACGTCAAGACGAGGGTAAAGAGAGAGTCCAATTCTCAAAG |
| P-tS1m- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | CAAAAAACAAAAAAAACAAAAAAAAAACCAAAAAAACAAAACACAATGGTGA |
| | GCAAGGGCGAGGAGCTGTTCACCGGGGTGGTGCCCATCCTGGTCGAGCTGGA |
| | CGGCGACGTAAACGGCCACAAGTTCAGCGTGTCTGGCGAGGGCGAGGGCGAT |
| | GCCACCTACGGCAAGCTGACCCTGAAGTTCATCTGCACCACCGGCAAGCTGC |
| | CCGTGCCCTGGCCCACCCTCGTGACCACCCTGACCTACGGCGTGCAGTGCTT |
| | CAGCCGCTACCCCGACCACATGAAGCAGCACGACTTCTTCAAGTCCGCCATG |
| | CCCGAAGGCTACGTCCAGGAGCGCACCATCTTCTTCAAGGACGACGGCAACT |
| | ACAAGACCCGCGCCGAGGTGAAGTTCGAGGGCGACACCCTGGTGAACCGCAT |
| | CGAGCTGAAGGGCATCGACTTCAAGGAGGACGGCAACATCCTGGGGCACAAG |
| | CTGGAGTACAACTACAACAGCCACAACGTCTATATCATGGCCGACAAGCAGA |
| | AGAACGGCATCAAGGCGAACTTCAAGATCCGCCACAACATCGAGGACGGCAG |
| | CGTGCAGCTCGCCGACCACTACCAGCAGAACACCCCCATCGGCGACGGCCCC |
| | GTGCTGCTGCCCGACAACCACTACCTGAGCACCCAGTCCGCCCTGAGCAAAG |
| | ACCCCAACGAGAAGCGCGATCACATGGTCCTGCTGGAGTTCGTGACCGCCGC |
| | CGGGATCACTCTCGGCATGGACGAGCTGTACAAGTAAAAAAAAAAAAAAAGC |
| | CCGGATAGCTCAGTCGGTAGAGCAGCGGCCTATGCGGCCGCCGACCAGAATC |
| | ATGCAAGTGCGTAAGATAGTCGCGGGTCGGCGGCCGCATTCGAGGCCGCGTC |
| | CAGGGTTCAAGTCCCTGTTCGGGCGCCACTGCAGAAAAAAAAAAAATTAAAA |
| | CAGCCTGTGGGTTGATCCCACCCACAGGCCCATTGGGCGCTAGCACTCTGGT |
| | ATCACGGTACCTTTGTGCGCCTGTTTTATACCCCCTCCCCCAACTGTAACTT |
| | AGAAGTAACACACACCGATCAACAGTCAGCGTGGCACACCAGCCACGTTTTG |
| | ATCAAGCACTTCTGTTACCCCGGACTGAGTATCAATAGACTGCTCACGCGGT |
| | TGAAGGAGAAAGCGTTCGTTATCCGGCCAACTACTTCGAAAAACCTAGTAAC |
| | ACCGTGGAAGTTGCAGAGTGTTTCGCTCAGCACTACCCCAGTGTAGATCAGG |
| | TCGATGAGTCACCGCATTCCCCACGGGCGACCGTGGCGGTGGCTGCGTTGGC |
| | GGCCTGCCCATGGGGAAACCCATGGGACGCTCTAATACAGACATGGTGCGAA |
| | GAGTCTATTGAGCTAGTTGGTAGTCCTCCGGCCCCTGAATGCGGCTAATCCT |
| | AACTGCGGAGCACACACCCTCAAGCCAGAGGGCAGTGTGTCGTAACGGGCAA |
| | CTCTGCAGCGGAACCGACTACTTTGGGTGTCCGTGTTTCATTTTATTCCTAT |
| | ACTGGCTGCTTATGGTGACAATTGAGAGATCGTTACCATATAGCTATTGGAT |
| | TGGCCATCCGGTGACTAATAGAGCTATTATATATCCCTTTGTTGGGTTTATA |
| | CCACTTAGCTTGAAAGAGGTTAAAACATTACAATTCATTGTTAAGTTGAATA |
| | CAGCAAAAAAAAACAAAAAACAAAACGGCTATTATGCGTTACCGGCGAGACG |
| | CTACGGACTTAAATAATTGAGCCTTAAAGAAGAAATTCTTTAAGTGGATGCT |
| | CTCAAACTCAGGGAAACCTAAATCTAGTTATAGACAAGGCAATCCTGAGCCA |
| | AGCCGAAGTAGTAATTAGTAAGACCAGTGGACAATCGACG |
| SEQ ID NO. | TAATACGACTCACTATAGGGGATCCGGGAGACCCTCGACCGTCGATTGTCCA |
| 42: | CTGGTCAACAATAGATGACTTACAACTAATCGGAAGGTGCAGAGACTCGACG |
| pML87_EGF | GGAGCTACCCTAACGTCAAGACGAGGGTAAAGAGAGAGTCCAATTCTCAAAG |
| P-tS1m- | CCAATAGGCAGTAGCGAAAGCTGCAAGAGAATGAAAATCCGTTGACCTTAAA |
| | CGGTCGTGTGGGTTCAAGTCCCTCCACCCCCACGCCGGAAACGCAATAGCCG |
| | CAAAAAACAAAAAAAACAAAAAAAAAACCAAAAAAAAAAACACAATGGTGAG |
| | CAAGGGCGAGGAGCTGTTCACCGGGGTGGTGCCCATCCTGGTCGAGCTGGAC |
| | GGCGACGTAAACGGCCACAAGTTCAGCGTGTCTGGCGAGGGCGAGGGCGATG |
| | CCACCTACGGCAAGCTGACCCTGAAGTTCATCTGCACCACCGGCAAGCTGCC |
| | CGTGCCCTGGCCCACCCTCGTGACCACCCTGACCTACGGCGTGCAGTGCTTC |
| | AGCCGCTACCCCGACCACATGAAGCAGCACGACTTCTTCAAGTCCGCCATGC |
| | CCGAAGGCTACGTCCAGGAGCGCACCATCTTCTTCAAGGACGACGGCAACTA |
| | CAAGACCCGCGCCGAGGTGAAGTTCGAGGGCGACACCCTGGTGAACCGCATC |
| | GAGCTGAAGGGCATCGACTTCAAGGAGGACGGCAACATCCTGGGGCACAAGC |
| | TGGAGTACAACTACAACAGCCACAACGTCTATATCATGGCCGACAAGCAGAA |
| | GAACGGCATCAAGGCGAACTTCAAGATCCGCCACAACATCGAGGACGGCAGC |
| | GTGCAGCTCGCCGACCACTACCAGCAGAACACCCCCATCGGCGACGGCCCCG |
| | TGCTGCTGCCCGACAACCACTACCTGAGCACCCAGTCCGCCCTGAGCAAAGA |
| | CCCCAACGAGAAGCGCGATCACATGGTCCTGCTGGAGTTCGTGACCGCCGCC |
| | GGGATCACTCTCGGCATGGACGAGCTGTACAAGTAACAAAAAACAAAAAAAA |
| | CAAAAAAAAAACCAAAAAAACAAAACACAAAAAAAAAAAAAAGCCCGGATAG |
| | CTCAGTCGGTAGAGCAGCGGCCTATGCGGCCGCCGACCAGAATCATGCAAGT |
| | GCGTAAGATAGTCGCGGGTCGGCGGCCGCATTCGAGGCCGCGTCCAGGGTTC |
| | AAGTCCCTGTTCGGGCGCCACTGCAGAAAAAAAAAAAACAAAAAACAAAAAA |
| | AACAAAAAAAAAACCAAAAAAACAAAACACATTAAAACAGCCTGTGGGTTGA |
| | TCCCACCCACAGGCCCATTGGGCGCTAGCACTCTGGTATCACGGTACCTTTG |
| | TGCGCCTGTTTTATACCCCCTCCCCCAACTGTAACTTAGAAGTAACACACAC |
| | CGATCAACAGTCAGCGTGGCACACCAGCCACGTTTTGATCAAGCACTTCTGT |
| | TACCCCGGACTGAGTATCAATAGACTGCTCACGCGGTTGAAGGAGAAAGCGT |
| | TCGTTATCCGGCCAACTACTTCGAAAAACCTAGTAACACCGTGGAAGTTGCA |
| | GAGTGTTTCGCTCAGCACTACCCCAGTGTAGATCAGGTCGATGAGTCACCGC |
| | ATTCCCCACGGGCGACCGTGGCGGTGGCTGCGTTGGCGGCCTGCCCATGGGG |
| | AAACCCATGGGACGCTCTAATACAGACATGGTGCGAAGAGTCTATTGAGCTA |
| | GTTGGTAGTCCTCCGGCCCCTGAATGCGGCTAATCCTAACTGCGGAGCACAC |
| | ACCCTCAAGCCAGAGGGCAGTGTGTCGTAACGGGCAACTCTGCAGCGGAACC |
| | GACTACTTTGGGTGTCCGTGTTTCATTTTATTCCTATACTGGCTGCTTATGG |
| | TGACAATTGAGAGATCGTTACCATATAGCTATTGGATTGGCCATCCGGTGAC |
| | TAATAGAGCTATTATATATCCCTTTGTTGGGTTTATACCACTTAGCTTGAAA |
| | GAGGTTAAAACATTACAATTCATTGTTAAGTTGAATACAGCAAAAAAAAACA |
| | AAAAACAAAACGGCTATTATGCGTTACCGGCGAGACGCTACGGACTTAAATA |
| | ATTGAGCCTTAAAGAAGAAATTCTTTAAGTGGATGCTCTCAAACTCAGGGAA |
| | ACCTAAATCTAGTTATAGACAAGGCAATCCTGAGCCAAGCCGAAGTAGTAAT |
| | TAGTAAGACCAGTGGACAATCGACG |
| SEQ ID NO. | TAATACGACTCACTATAGGGGATCCGGGAGACCCTCGACCGTCGATTGTCCA |
| 43: | CTGGTCAAAAAAAAAAAAAGCCCGGATAGCTCAGTCGGTAGAGCAGCGGCCT |
| pML89_tS1m | ATGCGGCCGCCGACCAGAATCATGCAAGTGCGTAAGATAGTCGCGGGTCGGC |
| -CVB3- | GGCCGCATTCGAGGCCGCGTCCAGGGTTCAAGTCCCTGTTCGGGCGCCACTG |
| | CAGAAAAAAAAAAAAAACAATAGATGACTTACAACTAATCGGAAGGTGCAGA |
| | GACTCGACGGGAGCTACCCTAACGTCAAGACGAGGGTAAAGAGAGAGTCCAA |
| | TTCTCAAAGCCAATAGGCAGTAGCGAAAGCTGCAAGAGAATGAAAATCCGTT |
| | GACCTTAAACGGTCGTGTGGGTTCAAGTCCCTCCACCCCCACGCCGGAAACG |
| | CAATAGCCGAAAAACAAAAAACAAAAAAAACAAAAAAAAAACCAAAAAAACA |
| | AAACACATTAAAACAGCCTGTGGGTTGATCCCACCCACAGGCCCATTGGGCG |
| | CTAGCACTCTGGTATCACGGTACCTTTGTGCGCCTGTTTTATACCCCCTCCC |
| | CCAACTGTAACTTAGAAGTAACACACACCGATCAACAGTCAGCGTGGCACAC |
| | CAGCCACGTTTTGATCAAGCACTTCTGTTACCCCGGACTGAGTATCAATAGA |
| | CTGCTCACGCGGTTGAAGGAGAAAGCGTTCGTTATCCGGCCAACTACTTCGA |
| | AAAACCTAGTAACACCGTGGAAGTTGCAGAGTGTTTCGCTCAGCACTACCCC |
| | AGTGTAGATCAGGTCGATGAGTCACCGCATTCCCCACGGGCGACCGTGGCGG |
| | TGGCTGCGTTGGCGGCCTGCCCATGGGGAAACCCATGGGACGCTCTAATACA |
| | GACATGGTGCGAAGAGTCTATTGAGCTAGTTGGTAGTCCTCCGGCCCCTGAA |
| | TGCGGCTAATCCTAACTGCGGAGCACACACCCTCAAGCCAGAGGGCAGTGTG |
| | TCGTAACGGGCAACTCTGCAGCGGAACCGACTACTTTGGGTGTCCGTGTTTC |
| | ATTTTATTCCTATACTGGCTGCTTATGGTGACAATTGAGAGATCGTTACCAT |
| | ATAGCTATTGGATTGGCCATCCGGTGACTAATAGAGCTATTATATATCCCTT |
| | TGTTGGGTTTATACCACTTAGCTTGAAAGAGGTTAAAACATTACAATTCATT |
| | GTTAAGTTGAATACAGCAAAATGGGAGTCAAAGTTCTGTTTGCCCTGATCTG |
| | CATCGCTGTGGCCGAGGCCAAGCCCACCGAGAACAACGAAGACTTCAACATC |
| | GTGGCCGTGGCCAGCAACTTCGCGACCACGGATCTCGATGCTGACCGCGGGA |
| | AGTTGCCCGGCAAGAAGCTGCCGCTGGAGGTGCTCAAAGAGATGGAAGCCAA |
| | TGCCCGGAAAGCTGGCTGCACCAGGGGCTGTCTGATCTGCCTGTCCCACATC |
| | AAGTGCACGCCCAAGATGAAGAAGTTCATCCCAGGACGCTGCCACACCTACG |
| | AAGGCGACAAAGAGTCCGCACAGGGGGGCATAGGCGAGGCGATCGTCGACAT |
| | TCCTGAGATTCCTGGGTTCAAGGACTTGGAGCCCATGGAGCAGTTCATCGCA |
| | CAGGTCGATCTGTGTGTGGACTGCACAACTGGCTGCCTCAAAGGGCTTGCCA |
| | ACGTGCAGTGTTCTGACCTGCTCAAGAAGTGGCTGCCGCAACGCTGTGCGAC |
| | CTTTGCCAGCAAGATCCAGGGCCAGGTGGACAAGATCAAGGGGGCCGGTGGT |
| | GACTAAAAAAAACAAAAAACAAAACGGCTATTATGCGTTACCGGCGAGACGC |
| | TACGGACTTAAATAATTGAGCCTTAAAGAAGAAATTCTTTAAGTGGATGCTC |
| | TCAAACTCAGGGAAACCTAAATCTAGTTATAGACAAGGCAATCCTGAGCCAA |
| | GCCGAAGTAGTAATTAGTAAAAAAAAAAAAAAAGCCCGGATAGCTCAGTCGG |
| | TAGAGCAGCGGCCTATGCGGCCGCCGACCAGAATCATGCAAGTGCGTAAGAT |
| | AGTCGCGGGTCGGCGGCCGCATTCGAGGCCGCGTCCAGGGTTCAAGTCCCTG |
| | TTCGGGCGCCACTGCAGAAAAAAAAAAAAGACCAGTGGACAATCGACG |

TABLE 3
DNA sequences encoding linear
RNA precursor and circular RNA elements

| SEQ ID NO/ | |
|---|---|
| Description | SEQUENCE |
| SEQ ID NO. | TAATACGACTCACTATAGG |
| 44: | |
| T7 promoter | |
| SEQ ID NO. | CGTCGATTGTCCACTGGTC |
| 45: | |
| 5′ external | |
| homology | |
| arm | |
| SEQ ID NO. | CGCCGGAAACGCAATAGCCG |
| 46: | |
| 5′ internal | |
| homology | |
| arm | |
| SEQ ID NO. | GGCTATTATGCGTTACCGGCG |
| 47: | |
| 3′ internal | |
| homology | |
| arm | |
| SEQ ID NO. | GACCAGTGGACAATCGACG |
| 48: | |
| 3′ external | |
| homology | |
| arm | |
| SEQ ID NO. | AACAATAGATGACTTACAACTAATCGGAAGGTGCAGAGACTCGACGGGAGCTACCCTA |
| 49: | ACGTCAAGACGAGGGTAAAGAGAGAGTCCAATTCTCAAAGCCAATAGGCAGTAGCGAA |
| Ana2.0 PIE | AGCTGCAAGAGAATGAAAATCCGTTGACCTTAAACGGTCGTGTGGGTTCAAGTCCCTC |
| 3′ | CACCCCCA |
| SEQ ID NO. | AGACGCTACGGACTTAAATAATTGAGCCTTAAAGAAGAAATTCTTTAAGTGGATGCTCT |
| 50: | CAAACTCAGGGAAACCTAAATCTAGTTATAGACAAGGCAATCCTGAGCCAAGCCGAAG |
| Ana2.0 PIE | TAGTAATTAGTAA |
| 5′ | |
| SEQ ID NO. | TTAAAACAGCCTGTGGGTTGATCCCACCCACAGGCCCATTGGGCGCTAGCACTCTGGT |
| 51: | ATCACGGTACCTTTGTGCGCCTGTTTTATACCCCCTCCCCCAACTGTAACTTAGAAGTA |
| CVB3 IRES | ACACACACCGATCAACAGTCAGCGTGGCACACCAGCCACGTTTTGATCAAGCACTTCT |
| | GTTACCCCGGACTGAGTATCAATAGACTGCTCACGCGGTTGAAGGAGAAAGCGTTCGT |
| | TATCCGGCCAACTACTTCGAAAAACCTAGTAACACCGTGGAAGTTGCAGAGTGTTTCG |
| | CTCAGCACTACCCCAGTGTAGATCAGGTCGATGAGTCACCGCATTCCCCACGGGCGA |
| | CCGTGGCGGTGGCTGCGTTGGCGGCCTGCCCATGGGGAAACCCATGGGACGCTCTA |
| | ATACAGACATGGTGCGAAGAGTCTATTGAGCTAGTTGGTAGTCCTCCGGCCCCTGAAT |
| | GCGGCTAATCCTAACTGCGGAGCACACACCCTCAAGCCAGAGGGCAGTGTGTCGTAA |
| | CGGGCAACTCTGCAGCGGAACCGACTACTTTGGGTGTCCGTGTTTCATTTTATTCCTAT |
| | ACTGGCTGCTTATGGTGACAATTGAGAGATCGTTACCATATAGCTATTGGATTGGCCAT |
| | CCGGTGACTAATAGAGCTATTATATATCCCTTTGTTGGGTTTATACCACTTAGCTTGAAA |
| | GAGGTTAAAACATTACAATTCATTGTTAAGTTGAATACAGCAAA |
| SEQ ID NO. | ATGCGGCCGCCGACCAGAATCATGCAAGTGCGTAAGATAGTCGCGGGTCGGCGGCC |
| 52: | GCAT |
| S1m aptamer | |
| SEQ ID NO. | ATGCGGCCGCCGACCAGAATCATGCAAGTGCGTAAGATAGTCGCGGGTCGGCGGCC |
| 53: | GCATCTGCTGGGAAGCTACGATCCGTAGAAAATGCGGCCGCCGACCAGAATCATGCA |
| 4xS1m | AGTGCGTAAGATAGTCGCGGGTCGGCGGCCGCATCTGCTGGGTAGCTGTGAACCGTA |
| aptamer | GAAAATGCGGCCGCCGACCAGAATCATGCAAGTGCGTAAGATAGTCGCGGGTCGGCG |
| | GCCGCATCTGCTGGGAAGCTACGATCCGTAGAAAATGCGGCCGCCGACCAGAATCAT |
| | GCAAGTGCGTAAGATAGTCGCGGGTCGGCGGCCGCAT |
| SEQ ID NO. | GCCCGGATAGCTCAGTCGGTAGAGCAGCGGCCTATGCGGCCGCCGACCAGAATCATG |
| 54: | CAAGTGCGTAAGATAGTCGCGGGTCGGCGGCCGCATTCGAGGCCGCGTCCAGGGTT |
| tRNA-S1m | CAAGTCCCTGTTCGGGCGCCA |
| aptamer | |
| SEQ ID NO. | ATGGTGAGCAAGGGCGAGGAGCTGTTCACCGGGGTGGTGCCCATCCTGGTCGAGCT |
| 55: | GGACGGCGACGTAAACGGCCACAAGTTCAGCGTGTCTGGCGAGGGCGAGGGCGATG |
| EGFP | CCACCTACGGCAAGCTGACCCTGAAGTTCATCTGCACCACCGGCAAGCTGCCCGTGC |
| | CCTGGCCCACCCTCGTGACCACCCTGACCTACGGCGTGCAGTGCTTCAGCCGCTACC |
| | CCGACCACATGAAGCAGCACGACTTCTTCAAGTCCGCCATGCCCGAAGGCTACGTCCA |
| | GGAGCGCACCATCTTCTTCAAGGACGACGGCAACTACAAGACCCGCGCCGAGGTGAA |
| | GTTCGAGGGCGACACCCTGGTGAACCGCATCGAGCTGAAGGGCATCGACTTCAAGGA |
| | GGACGGCAACATCCTGGGGCACAAGCTGGAGTACAACTACAACAGCCACAACGTCTAT |
| | ATCATGGCCGACAAGCAGAAGAACGGCATCAAGGCGAACTTCAAGATCCGCCACAACA |
| | TCGAGGACGGCAGCGTGCAGCTCGCCGACCACTACCAGCAGAACACCCCCATCGGC |
| | GACGGCCCCGTGCTGCTGCCCGACAACCACTACCTGAGCACCCAGTCCGCCCTGAGC |
| | AAAGACCCCAACGAGAAGCGCGATCACATGGTCCTGCTGGAGTTCGTGACCGCCGCC |
| | GGGATCACTCTCGGCATGGACGAGCTGTACAAGTAA |
| SEQ ID NO. | ATGGGAGTCAAAGTTCTGTTTGCCCTGATCTGCATCGCTGTGGCCGAGGCCAAGCCCA |
| 56: | CCGAGAACAACGAAGACTTCAACATCGTGGCCGTGGCCAGCAACTTCGCGACCACGG |
| hGLuc | ATCTCGATGCTGACCGCGGGAAGTTGCCCGGCAAGAAGCTGCCGCTGGAGGTGCTCA |
| | AAGAGATGGAAGCCAATGCCCGGAAAGCTGGCTGCACCAGGGGCTGTCTGATCTGCC |
| | TGTCCCACATCAAGTGCACGCCCAAGATGAAGAAGTTCATCCCAGGACGCTGCCACAC |
| | CTACGAAGGCGACAAAGAGTCCGCACAGGGGGGCATAGGCGAGGCGATCGTCGACAT |
| | TCCTGAGATTCCTGGGTTCAAGGACTTGGAGCCCATGGAGCAGTTCATCGCACAGGTC |
| | GATCTGTGTGTGGACTGCACAACTGGCTGCCTCAAAGGGCTTGCCAACGTGCAGTGTT |
| | CTGACCTGCTCAAGAAGTGGCTGCCGCAACGCTGTGCGACCTTTGCCAGCAAGATCC |
| | AGGGCCAGGTGGACAAGATCAAGGGGGCCGGTGGTGACTAA |
| SEQ ID NO. | AGAGCGGCCGCTTTTTCAGCAAGATTAAGCCCAGGGCAGAGCCATCTATTGCTTACAT |
| 57: | TTGCTTCTGACACAACTGTGTTCACTAGCAACCTCAAACAGACACC |
| 5′ UTR | |
| SEQ ID NO. | AGCTCGCTTTCTTGCTGTCCAATTTCTATTAAAGGTTCCTTTGTTCCCTAAGTCCAACTA |
| 58: | CTAAACTGGGGGATATTATGAAGGGCCTTGAGCATCTGGATTCTGCCTAATAAAAAACA |
| 3′ UTR | TTTATTTTCATTGCAGCTCGCTTTCTTGCTGTCCAATTTCTATTAAAGGTTCCTTTGTTC |
| | CCTAAGTCCAACTACTAAACTGGGGGATATTATGAAGGGCCTTGAGCATCTGGATTCT |
| | GCCTAATAAAAAACATTTATTTTCATTGC |
| SEQ ID NO. | AAAAACAAAAAACAAAAAAAACAAAAAAAAAACCAAAAAAACAAAACACA |
| 59: | |
| 5′ spacer | |
| poly(A) site 1 | |
| 50 bp | |
| SEQ ID NO. | AAAAAACAAAAAACAAAAC |
| 60: | |
| 3′spacer | |
| poly(A) site 2 | |
| 19 bp | |
| SEQ ID NO. | AACAATAGATGACTTACAACTAATCGGAAGGTGCAGAGACTCGACGGGAGCTACCCTA |
| 61: | ACGTCAAGACGAGGGTAAAGAGAGAGTCCAATTCTCAAAGCCAATAGGCAGTAGCGAA |
| Ana2.0 3′ | AGCTGCAAGAGAATG |
| intron | |
| SEQ ID NO. | AAAATCCGTTGACCTTAAACGGTCGTGTGGGTTCAAGTCCCTCCACCCCCA |
| 62: | |
| Ana2.0 3′ | |
| exon | |
| SEQ ID NO. | AAATAATTGAGCCTTAAAGAAGAAATTCTTTAAGTGGATGCTCTCAAACTCAGGGAAAC |
| 63: | CTAAATCTAGTTATAGACAAGGCAATCCTGAGCCAAGCCGAAGTAGTAATTAGTAA |
| Ana2.0 5′ | |
| intron | |
| SEQ ID NO. | AGACGCTACGGACTT |
| 64: | |
| Ana2.0 5′ | |
| exon | |

TABLE 4
RNA sequences of linear RNA precursor and circular RNA elements

| SEQ ID NO/ | |
|---|---|
| Description | SEQUENCE |
| SEQ ID NO. | UAAUACGACUCACUAUAGG |
| 68: | |
| T7 promoter | |
| SEQ ID NO. | CGUCGAUUGUCCACUGGUC |
| 69: | |
| 5′ external | |
| homology | |
| arm | |
| SEQ ID NO. | CGCCGGAAACGCAAUAGCCG |
| 70: | |
| 5′ internal | |
| homology | |
| arm | |
| SEQ ID NO. | GGCUAUUAUGCGUUACCGGCG |
| 71: | |
| 3′ internal | |
| homology | |
| arm | |
| SEQ ID NO. | GACCAGUGGACAAUCGACG |
| 72: | |
| 3′ external | |
| homology | |
| arm | |
| SEQ ID NO. | AACAAUAGAUGACUUACAACUAAUCGGAAGGUGCAGAGACUCGACGGGAGCUACCC |
| 73: | UAACGUCAAGACGAGGGUAAAGAGAGAGUCCAAUUCUCAAAGCCAAUAGGCAGUAG |
| Ana2.0 PIE | CGAAAGCUGCAAGAGAAUGAAAAUCCGUUGACCUUAAACGGUCGUGUGGGUUCAAG |
| 3′ | UCCCUCCACCCCCA |
| SEQ ID NO. | AGACGCUACGGACUUAAAUAAUUGAGCCUUAAAGAAGAAAUUCUUUAAGUGGAUGCU |
| 74: | CUCAAACUCAGGGAAACCUAAAUCUAGUUAUAGACAAGGCAAUCCUGAGCCAAGCCG |
| Ana2.0 PIE | AAGUAGUAAUUAGUAA |
| 5′ | |
| SEQ ID NO. | UUAAAACAGCCUGUGGGUUGAUCCCACCCACAGGCCCAUUGGGCGCUAGCACUCUG |
| 75: | GUAUCACGGUACCUUUGUGCGCCUGUUUUAUACCCCCUCCCCCAACUGUAACUUAG |
| CVB3 IRES | AAGUAACACACACCGAUCAACAGUCAGCGUGGCACACCAGCCACGUUUUGAUCAAGC |
| | ACUUCUGUUACCCCGGACUGAGUAUCAAUAGACUGCUCACGCGGUUGAAGGAGAAA |
| | GCGUUCGUUAUCCGGCCAACUACUUCGAAAAACCUAGUAACACCGUGGAAGUUGCA |
| | GAGUGUUUCGCUCAGCACUACCCCAGUGUAGAUCAGGUCGAUGAGUCACCGCAUUC |
| | CCCACGGGCGACCGUGGCGGUGGCUGCGUUGGCGGCCUGCCCAUGGGGAAACCCA |
| | UGGGACGCUCUAAUACAGACAUGGUGCGAAGAGUCUAUUGAGCUAGUUGGUAGUCC |
| | UCCGGCCCCUGAAUGCGGCUAAUCCUAACUGCGGAGCACACACCCUCAAGCCAGAG |
| | GGCAGUGUGUCGUAACGGGCAACUCUGCAGCGGAACCGACUACUUUGGGUGUCCG |
| | UGUUUCAUUUUAUUCCUAUACUGGCUGCUUAUGGUGACAAUUGAGAGAUCGUUACC |
| | AUAUAGCUAUUGGAUUGGCCAUCCGGUGACUAAUAGAGCUAUUAUAUAUCCCUUUG |
| | UUGGGUUUAUACCACUUAGCUUGAAAGAGGUUAAAACAUUACAAUUCAUUGUUAAGU |
| | UGAAUACAGCAAA |
| SEQ ID NO. | AGAGCGGCCGCUUUUUCAGCAAGAUUAAGCCCAGGGCAGAGCCAUCUAUUGCUUAC |
| 76: | AUUUGCUUCUGACACAACUGUGUUCACUAGCAACCUCAAACAGACACC |
| 5′ UTR | |
| SEQ ID NO. | AGCUCGCUUUCUUGCUGUCCAAUUUCUAUUAAAGGUUCCUUUGUUCCCUAAGUCCA |
| 77: | ACUACUAAACUGGGGGAUAUUAUGAAGGGCCUUGAGCAUCUGGAUUCUGCCUAAUA |
| 3′ UTR | AAAAACAUUUAUUUUCAUUGCAGCUCGCUUUCUUGCUGUCCAAUUUCUAUUAAAGGU |
| | UCCUUUGUUCCCUAAGUCCAACUACUAAACUGGGGGAUAUUAUGAAGGGCCUUGAG |
| | CAUCUGGAUUCUGCCUAAUAAAAAACAUUUAUUUUCAUUGC |
| SEQ ID NO. | AAAAACAAAAAACAAAAAAAACAAAAAAAAAACCAAAAAAACAAAACACA |
| 78: | |
| 5′ spacer | |
| poly(A) site 1 | |
| 50 bp | |
| SEQ ID NO. | AAAAAACAAAAAACAAAAC |
| 79: | |
| 3′spacer | |
| poly(A) site 2 | |
| 19 bp | |
| SEQ ID NO. | AACAAUAGAUGACUUACAACUAAUCGGAAGGUGCAGAGACUCGACGGGAGCUACCC |
| 80: | UAACGUCAAGACGAGGGUAAAGAGAGAGUCCAAUUCUCAAAGCCAAUAGGCAGUAG |
| Ana2.0 3′ | CGAAAGCUGCAAGAGAAUG |
| intron | |
| SEQ ID NO. | AAAAUCCGUUGACCUUAAACGGUCGUGUGGGUUCAAGUCCCUCCACCCCCA |
| 81: | |
| Ana2.0 3′ | |
| exon | |
| SEQ ID NO. | AAAUAAUUGAGCCUUAAAGAAGAAAUUCUUUAAGUGGAUGCUCUCAAACUCAGGGAA |
| 82: | ACCUAAAUCUAGUUAUAGACAAGGCAAUCCUGAGCCAAGCCGAAGUAGUAAUUAGUA |
| Ana2.0 5′ | A |
| intron | |
| SEQ ID NO. | AGACGCUACGGACUU |
| 83: | |
| Ana2.0 5′ | |
| exon | |

1. A circular RNA comprising a protein coding region and at least one RNA aptamer.

2. The circular RNA of claim 1, wherein;
the at least one RNA aptamer binds to an affinity ligand;
the affinity ligand comprises protein A, protein G, streptavidin, glutathione, dextran, a fluorescent molecule, or 6×His;
the affinity ligand comprises streptavidin; and/or
the affinity ligand is immobilized on a chromatography resin.

3-5. (canceled)

6. The circular RNA of claim 1, wherein;
the RNA aptamer is S1m, Sm, or a derivative or fragment thereof;
the circular RNA comprises between one to four RNA aptamers;
the RNA aptamers are identical;
at least one of the RNA aptamers is distinct;
the RNA aptamer is synthetically derived;
the RNA aptamer is a split aptamer or an X-aptamer;
the RNA aptamer is naturally-derived;
the RNA aptamer is derived from a hairpin RNA, a tRNA, or a riboswitch;
the RNA aptamer comprises the nucleotide sequence of SEQ ID NO: 65 or 66;
the RNA aptamer is about 30-200 nucleotides in length
the RNA aptamer is about 50-200 nucleotides in length
the RNA aptamer is not a histone stem-loop;
the RNA aptamer does not bind eIF4G;
the RNA aptamer is embedded in an RNA scaffold;
the RNA scaffold comprises at least one secondary structure motif;
the secondary structure motif is a tetraloop, a pseudoknot, or a stem-loop;
the RNA scaffold comprises at least one tertiary structure;
the secondary structure motif and/or tertiary structure are nuclease resistant;
the RNA scaffold comprises a transfer RNA (tRNA);
the RNA aptamer is embedded in a tRNA hairpin loop of the tRNA;
the RNA aptamer is embedded in a tRNA anticodon loop of the tRNA;
the RNA aptamer is embedded in a tRNA D loop of the tRNA; and/or
the RNA aptamer embedded tRNA comprises the nucleotide sequence of SEQ ID NO: 67
an internal ribosome entry site (IRES) is positioned at the 5′ end of the protein coding region;
an IRES is positioned at the 3′ end of the protein coding region;
the IRES is derived from Coxsackievirus B3 (CVB3), Encephalomyocarditis virus (EMCV), Dicistroviruses, hepatitis C virus (HCV), poliovirus (PV), enterovirus 71 (EV71), human rhinovirus (HRV), foot-and-mouth disease virus (FMDV), or synthetic IRES; and/or
the IRES comprises a polynucleotide sequence of SEQ ID NO: 75.

7-32. (canceled)

33. The circular RNA of claim 1, wherein the protein coding region encodes at least one polypeptide or peptide, optionally wherein the polypeptide is a biologically active polypeptide, a therapeutic polypeptide, or an antigenic polypeptide.

34. (canceled)

35. The circular RNA of claim 1, wherein;
the circular RNA comprises at least one 5′ internal homology arm and at least one 3′ internal homology arm;
the 5′ internal homology arm is about 5 to about 50 nucleotides in length;
the 5′ internal homology arm comprises the nucleotide sequence of SEQ ID NO: 70;
the 3′ internal homology arm is about 5 to about 50 nucleotides in length;
the 3′ internal homology arm comprises the nucleotide sequence of SEQ ID NO: 71;
the circular RNA comprises at least one 3′ exon element;
the 3′ exon element comprises the nucleotide sequence of SEQ ID NO: 81;
wherein the circular RNA comprises at least one 5′ exon element;
the 5′ exon element comprises the nucleotide sequence of SEQ ID NO: 83;
the circular RNA comprises at least one spacer sequence;
the spacer sequence is about 5 to about 75 nucleotides in length;
the spacer sequence comprises the nucleotide sequence of SEQ ID NO: 78 or 79;
the spacer sequence is positioned at one or both of a 5′ end and 3′ end of any one of the following elements: the protein coding region, the IRES, the 5′ internal homology arm, the 3′ internal homology arm, the 5′ exon element, and the 3′ exon element;
the circular RNA comprises the following elements, from 5′ to 3′: a) the 3′ exon element, b) the 5′ internal homology arm, c) the spacer sequence, d) the IRES, e) the protein coding region, f) the spacer sequence, g) the 3′ internal homology arm, and h) the 5′ exon element;
the circular RNA comprises the following elements, from 5′ to 3′: a) the 3′ exon element, b) the 5′ internal homology arm, c) the spacer sequence, d) the protein coding region, e) the IRES, f) the spacer sequence, g) the 3′ internal homology arm, and h) the 5′ exon element; and/or
the at least one RNA aptamer is positioned at a 5′ end or a 3′ end of any one of elements a)-h).

36-50. (canceled)

51. The circular RNA of claim 1, wherein;
the circular RNA contains at least one 5′ untranslated region (5′ UTR), at least one 3′ untranslated region (3′ UTR), and/or at least one polyadenylation (polyA) sequence;
the 5′ UTR, the 3′ UTR, and/or the poly A sequence are spacer sequences;
the at least one RNA aptamer is positioned: a) before the 3′ exon element, b) between the 3′ exon element and the 5′ internal homology arm, c) between the 5′ internal homology arm and the 5′ spacer sequence, d) between the 5′ spacer sequence and the IRES, e) between the protein coding region and the 3′ spacer sequence, f) between the 3′ spacer sequence and the 3′ internal homology arm, g) between the 3′ internal homology arm and the 5′ exon element, h) after the 5′ exon element, i) between the 3′ exon and the IRES, and/or i) between the IRES and the 5′ exon element; and/or
the at least one RNA aptamer is positioned: a) before the 3′ exon element, b) between the 3′ exon element and the 5′ internal homology arm, c) between the 5′ internal homology arm and the 5′ spacer sequence, d) between the 5′ spacer sequence and the protein coding region, e) between the IRES and the 3′ spacer sequence, f) between the 3′ spacer sequence and the 3′ internal homology arm, g) between the 3′ internal homology arm and the 5′ exon element, h) after the 5′ exon element, i) between the 3′ exon and the protein coding region, and/or j) between the protein coding region and the 5′ exon element.

52-54. (canceled)

55. The circular RNA of claim 1, wherein
the circular RNA comprises at least one chemical modification;
the chemical modification is pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, 2′-O-methyl uridine, or N6-methyladenosine;
the chemical modification is pseudouridine, N1-methylpseudouridine, 5-methylcytosine, 5-methoxyuridine, N6-methyladenosine or a combination thereof; and/or
the chemical modification is N1-methylpseudouridine.

56-58. (canceled)

59. A linear precursor RNA comprising at least a self-splicing ribozyme and a protein coding region, wherein the linear precursor RNA comprises at least one RNA aptamer.

60-86. (canceled)

87. The linear precursor RNA of claim 59, wherein:
the self-splicing ribozyme comprises at least two catalytic subunits;
the self-splicing ribozyme catalytic subunits derive from either a group I intron or a group II intron RNA transcript or a fragment thereof;
the self-splicing ribozyme catalytic subunits derive from a permuted intron-exon (PIE) sequence from Cyanobacterium anabaena pre-tRNA-Leu gene, T4 phage Td gene, or Tetrahymena pre-rRNA;
the catalytic activity of the two subunits results in a circularized RNA; and/or
the linear precursor RNA is synthesized using in vitro transcription (IVT).

88-90. (canceled)

91. The linear precursor RNA of claim 59, wherein:
the linear precursor RNA comprises the following elements, from 5′ to 3′: a) a 5′ external homology arm, b) a 3′ self-splicing PIE fragment, c) a 5′ internal homology arm, d) a 5′ spacer sequence, e) an internal ribosome entry site (IRES) f) a protein coding region, g) a 3′ spacer sequence, h) a 3′ internal homology arm, i) a 5′ self-splicing PIE fragment, and j) a 3′ external homology arm, wherein the RNA aptamer is present at one or both of the 5′ end or 3′ end of any one of elements a)-j); or
the linear precursor RNA comprises the following elements, from 5′ to 3′: a) a 5′ external homology arm, b) a 3′ self-splicing PIE fragment, c) a 5′ internal homology arm, d) a 5′ spacer sequence, e) a protein coding region, f) an IRES, g) a 3′ spacer sequence, h) a 3′ internal homology arm, i) a 5′ self-splicing PIE fragment, and i) a 3′ external homology arm, wherein the RNA aptamer is present at one or both of the 5′ end or 3′ end of any one of elements a)-j), and
optionally wherein
the 5′ external homology arm and the 3′ external homology arm are each independently about 5 to about 50 nucleotides in length, the 5′ external homology arm and the 3′ external homology arm comprises the nucleotide sequence of SEQ ID NO: 69 or SEQ ID NO: 72,
the 5′ self-splicing PIE fragment comprises the nucleotide sequence of SEQ ID NO: 74;
the 5′ internal homology arm is about 5 to about 50 nucleotides in length;
the 5′ internal homology arm comprises the nucleotide sequence of SEQ ID NO: 70;
the 5′ spacer and the 3′ spacer are each independently about 5 to 75 nucleotides in length;
the 5′ spacer and the 3′ spacer comprises the nucleotide sequence of SEQ ID NO: 78 or SEQ ID NO: 79;
the 3′ self-splicing PIE fragment comprises the nucleotide sequence of SEQ ID NO: 73;
the IRES is derived from Coxsackievirus B3 (CVB3), Encephalomyocarditis virus (EMCV), Dicistroviruses, hepatitis C virus (HCV), poliovirus (PV), enterovirus 71 (EV71), human rhinovirus (HRV), foot-and-mouth disease virus (FMDV), or synthetic IRES; and/or
the IRES comprises the nucleotide sequence of SEQ ID NO: 75.

92-102. (canceled)

103. The linear precursor RNA of claim 59, wherein:
the linear precursor RNA comprises at least one 5′ untranslated region (5′ UTR), at least one 3′ untranslated region (3′ UTR), and/or a polyadenylation (polyA) sequence;
the protein coding region encodes at least one polypeptide;
the polypeptide is a biologically active polypeptide, a therapeutic polypeptide, or an antigenic polypeptide;
the RNA aptamer is a split aptamer comprising a 5′ portion and a 3′ portion;
the 5′ portion of the split aptamer is positioned 3′ of the 5′ exon element and the 3′ portion of the split aptamer is positioned 5′ of the 3′ exon element;
the 5′ portion of the split aptamer is positioned 3′ of the 3′ internal homology arm and the 3′ portion of the split aptamer is positioned 5′ of the 5′ internal homology arm;
the split aptamer is reformed to a functional aptamer upon circularization of the linear precursor RNA;
the at least one RNA aptamer is positioned: a) before the 5′ external homology arm, b) between the 5′ external homology arm and the 3′ self-splicing PIE fragment, c) between the 3′ self-splicing PIE fragment and the 5′ internal homology arm, d) between the 5′ internal homology arm and the 5′ spacer sequence, e) between the 5′ space sequence and the IRES, f) after the protein coding region but before the 3′ spacer sequence, g) between the 3′ spacer sequence and the 3′ internal homology arm, h) between the 3′ internal homology arm and the 5′ self-splicing PIE fragment, i) between the 5′ self-splicing PIE fragment and the 3′ external homology arm, and/or j) after the 3′ external homology arm; and/or
the at least one RNA aptamer is positioned: a) before the 5′ external homology arm, b) between the 5′ external homology arm and the 3′ self-splicing PIE fragment, c) between the 3′ self-splicing PIE fragment and the 5′ internal homology arm, d) between the 5′ internal homology arm and the 5′ spacer sequence, e) between the 5′ space sequence and the protein coding region, f) after the IRES but before the 3′ spacer sequence, g) between the 3′ spacer sequence and the 3′ internal homology arm, h) between the 3′ internal homology arm and the 5′ self-splicing PIE fragment, i) between the 5′ self-splicing PIE fragment and the 3′ external homology arm, and/or j) after the 3′ external homology arm.

104-116. (canceled)

117. A circular RNA comprising a protein coding region and at least one RNA aptamer, wherein the circular RNA is formed from the linear precursor RNA of claim 59 or a circular RNA comprising a protein coding region, wherein the circular RNA is formed from the linear precursor RNA of claim 59, and wherein the circular RNA lacks an RNA aptamer.

118. (canceled)

119. A nucleic acid that encodes the linear precursor RNA of claim 59.

120. (canceled)

121. A pharmaceutical composition comprising the circular RNA of claim 1.

122. A method of producing a circular RNA, comprising incubating the linear precursor RNA of claim 59 under conditions that result in the circularization of the linear precursor RNA, optionally wherein:
the linear precursor RNA is incubated with GTP and Mg2+;
the linear precursor RNA is incubated with GTP and Mg2+ for a time sufficient to circularize the linear precursor RNA;
the GTP is present at a concentration of about 1 mM to about 15 mM;
the GTP is present at a concentration of about 2 mM;
the Mg2+ is present at a concentration of about 1 mM to about 50 mM; and/or
the Mg2+ is present at a concentration of about 10 mM.

123-128. (canceled)

129. A method of producing a plurality of circular RNA molecules, comprising incubating a plurality of linear precursor RNA molecules under conditions that result in the circularization of at least a portion of the linear precursor RNA molecules, wherein each linear precursor RNA molecule comprises the linear precursor RNA of claim 59, optionally wherein at least about 30% of the linear precursor RNA molecules in the plurality are circularized.

130. (canceled)

131. A method for purifying a circular RNA, comprising the steps of:
(a) contacting a sample comprising the circular RNA of claim 1 with an affinity ligand that is immobilized on a chromatography resin, wherein the RNA aptamer comprises binding affinity for the affinity ligand; (b) eluting the circular RNA from the chromatography resin; and (c) purifying the circular RNA from the sample, optionally wherein the method comprises one or more washing steps between the contacting step (a) and the eluting step (b).

132. A method for purifying a linear precursor RNA, comprising the steps of:
(a) contacting a sample comprising the linear precursor RNA of claim 59 with an affinity ligand that is immobilized on a chromatography resin, wherein the RNA aptamer comprises binding affinity for the affinity ligand; (b) eluting the linear precursor RNA from the chromatography resin; and (c) purifying the linear precursor RNA from the sample, optionally wherein the method comprises one or more washing steps between the contacting step (a) and the eluting step (b).

133. (canceled)

134. A method of purifying a circular RNA, comprising the steps of:
(a) contacting a sample comprising the circular RNA with an affinity ligand that is immobilized on a chromatography resin; (b) eluting the circular RNA from the chromatography resin; and (c) isolating the circular RNA from the sample, wherein the circular RNA comprises a protein coding region and at least one RNA aptamer, wherein the RNA aptamer comprises binding affinity for the affinity ligand;
(a) contacting a sample comprising the linear precursor RNA with an affinity ligand that is immobilized on a chromatography resin; (b) eluting the linear precursor RNA from the chromatography resin; and (c) isolating the linear precursor RNA from the sample, wherein the linear precursor RNA comprises a protein coding region and at least one RNA aptamer, wherein the RNA aptamer comprises binding affinity for the affinity ligand; or
(a) contacting a sample comprising a plurality of linear precursor RNA molecules and a plurality of circular RNA molecules with an affinity ligand that is immobilized on a chromatography resin; and (b) isolating the circular RNA molecules from the sample, wherein the linear precursor RNA molecules comprise a protein coding region and at least one RNA aptamer and wherein the RNA aptamer comprises binding affinity for the affinity ligand, and wherein the circular RNA molecules lack an RNA aptamer,
optionally wherein the circular RNA molecules do not bind the affinity ligand and/or the circular RNA or linear precursor RNA is greater than or equal to 90% pure.

135-138. (canceled)

139. A method of treating or preventing a disease or disorder, comprising administering to a subject in need thereof the pharmaceutical composition of claim 121.

140. A pharmaceutical composition comprising a plurality of circular RNA molecules, wherein at least about 90% of the circular RNA comprise a protein coding region and at least one RNA aptamer.
